(19) United States
(12) Patent Application Publication
(10) Pub. No.: US 2026/0193177 A1
(43) Pub. Date: Jul. 9, 2026

(54) HYPERVALENT IODINE COMPOUND, RESIST COMPOSITION, LAMINATE OBTAINED FROM THE RESIST COMPOSITION, AND PATTERNING PROCESS

(71) Applicant: SHIN-ETSU CHEMICAL CO., LTD., Tokyo (JP)

(72) Inventors: Kousuke Ohyama, Joetsu-shi (JP); Shun Kikuchi, Joetsu-shi (JP); Satoshi Kusama, Joetsu-shi (JP); Ryo Nishikawa, Joetsu-shi (JP); Masaki Ohashi, Joetsu-shi (JP)

(73) Assignee: SHIN-ETSU CHEMICAL CO., LTD., Tokyo (JP)

(21) Appl. No.: 19/435,905

(22) Filed: Dec. 30, 2025

(30) Foreign Application Priority Data
Jan. 6, 2025 (JP) 2025-002008

(51) Int. Cl. C07C 381/12; G03F 7/00; G03F 7/004; G03F 7/11

(52) U.S. Cl. CPC C07C 381/12; G03F 7/0045; G03F 7/11; G03F 7/70033

(57) ABSTRACT

The present invention is a hypervalent iodine compound having a structure represented by the following general formula (1), where R¹ and R² each independently represent a halogen atom or a hydrocarbyl group having 1 to 10 carbon atoms and optionally containing a heteroatom, the R¹ and the R² optionally being bonded to each other to form a ring together with the carbon atoms bonded thereto and the atoms between the carbon atoms. This can provide: a non-chemically amplified resist composition excellent in sensitivity and resolution in lithography using a high-energy beam, especially electron beam (EB) lithography and EUV lithography; a laminate obtained from the resist composition; and a patterning process using the resist composition.

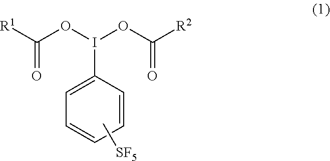

### TECHNICAL FIELD

The present invention relates to: a hypervalent iodine compound; a resist composition; a laminate obtained from the resist composition; and a patterning process.

### BACKGROUND ART

While a higher integration density, higher operating speed and lower power consumption of LSIs are demanded to comply with the expanding IoT market, the effort to reduce the pattern rule is in rapid progress. In particular, logic devices drive forward the miniaturization technology. As the advanced miniaturization technology, devices of 10-nm node are manufactured in a mass scale by the double, triple or quadro-patterning version of the immersion ArF lithography. Furthermore, the experimental mass-scale manufacture of 7-nm node devices by the next-generation extreme ultraviolet ray (EUV) lithography of wavelength 13.5 nm has started.

As miniaturization advances, image blurs due to acid diffusion are regarded as a problem (Non Patent Document 1). In order to ensure resolution for fine patterns with a post-45 nm size, it is suggested that not only the enhancement of dissolution contrast, which has been proposed previously, but also the controlling of acid diffusion is important (Non Patent Document 2). In chemically amplified resist materials, however, the sensitivity and the contrast are enhanced by acid diffusion. Accordingly, an attempt to minimize acid diffusion by lowering the temperature of post-exposure baking (PEB) and shortening the PEB time lowers the sensitivity and contrast markedly.

It is effective to control the acid diffusion by adding an acid generator that generates a bulky acid. Accordingly, it has been proposed to copolymerize a polymer with an acid generator in the form of an onium salt having polymerizable olefin. In post-16 nm size patterning of resist films, however, it is considered that patterning is impossible with chemically amplified resist films in view of the acid diffusion. Accordingly, development of a non-chemically amplified resist material is desired.

Examples of non-chemically amplified resist materials include polymethyl methacrylate (PMMA). PMMA is a positive resist material whose solubility in an organic solvent developer increases due to decreased molecular weight caused by scission of the main chain by EUV irradiation.

Hydrogensilsesquioxane (HSQ) is a negative resist material which turns insoluble in an alkaline developer through crosslinking by condensation reaction of silanol generated by EUV irradiation. Calixarene substituted with chlorine also functions as a negative resist material. These negative resist materials have a small molecular size prior to crosslinking and are free from causing blurs due to acid diffusion, and therefore, exhibit smaller edge roughness and very high resolution. Accordingly, the materials have been used as a pattern transfer material to show the resolution limit of the exposure apparatus. These materials, however, are insufficient in sensitivity, and further improvement is required.

The number of photons in EUV exposure being small is a factor that causes difficulties in developing materials for EUV lithography. The energy of EUV is much higher than that of an ArF excimer laser beam, and the number of photons in EUV exposure is 1/14 of that of ArF exposure. Furthermore, the size of the pattern formed by EUV exposure is half of that in ArF exposure or less. Therefore, EUV exposure is easily affected by variation in the number of photons. The variation in the number of photons in a radiation light region of extremely short wavelengths is the physical phenomenon of shot noise, and it is impossible to eliminate the influence of the variation. Therefore, so-called probability theory (stochastics) is attracting attention. The influence of shot noise cannot be eliminated, but there is discussion of how to reduce this influence. Due to the influence of shot noise, not only are critical dimension uniformity (CDU) and line width roughness (LWR) increased, a phenomenon that a hole gets blocked at a probability of one to several millions is observed. If a hole gets blocked, conduction failure occurs and the transistor does not function, and the performance of the entire device is adversely affected. Considering application for resists at a practical sensitivity, resists that mainly contain the above-described PMMA or HSQ are greatly affected by stochastics, and cannot achieve the desired resolution performance.

The introduction of an element that greatly absorbs EUV is attracting attention as a means for reducing the influence of shot noise on the side of the resist. Patent Document 1 proposes a chemically amplified resist composition containing bismuth atoms, which highly absorb EUV light. However, as stated earlier, a chemically amplified resist cannot realize excellent resolution performance in EUV lithography, in which the processed size is to be further miniaturized in the future.

Patent Document 2 proposes an organic solvent negative resist composition containing a tin compound. This composition mainly contains the element tin, which highly absorbs EUV light, and therefore, stochastics is improved, and high sensitivity and high resolution can be realized. However, such a so-called metal resist has many problems such as insufficient solubility in a solvent for resists, insufficient storage stability due to reactivity being too high, and defects due to residues after etching.

### CITATION LIST

### Patent Literature

Patent Document 1: JP2018-005224A
Patent Document 2: JP2021-503482A

### Non Patent Literature

Non Patent Document 1: SPIE Vol. 5039 p 1 (2003)
Non Patent Document 2: SPIE Vol. 6520 p 65203L-1 (2007)

### SUMMARY OF INVENTION

### Technical Problem

The present invention has been made in view of the above-described circumstances, and an object thereof is to provide: a non-chemically amplified resist composition excellent in sensitivity and resolution in lithography using a high-energy beam, especially electron beam (EB) lithography and EUV lithography; a laminate obtained from the resist composition; and a patterning process using the resist composition.

### Solution to Problem

To achieve the object, the present invention provides a hypervalent iodine compound having a structure represented by the following general formula (1),

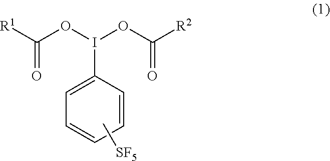

wherein R¹ and R² each independently represent a halogen atom or a hydrocarbyl group having 1 to 10 carbon atoms and optionally containing a heteroatom, the R¹ and the R² optionally being bonded to each other to form a ring together with the carbon atoms bonded thereto and the atoms between the carbon atoms.

A resist composition containing such a hypervalent iodine compound, having an SF₅ group, is excellent in sensitivity in high-energy beam, especially electron beam (EB) lithography and EUV lithography, and has enhanced solubility in a solvent. Thus, patterning excellent in sensitivity and roughness resolution is possible.

The present invention can also be a resist composition comprising: the above-described hypervalent iodine compound; a carboxy-group-containing compound; and a solvent.

Such a resist composition is excellent in sensitivity and resolution in high-energy beam, especially electron beam (EB) lithography and EUV lithography.

In this case, the carboxy-group-containing compound is preferably a polymer including a repeating unit represented by the following general formula (2) and/or a compound represented by the following general formula (3).

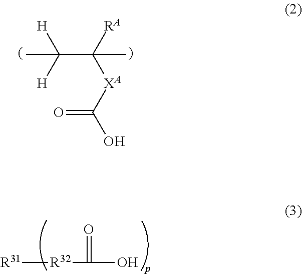

In the formulae, R^{A} represents a hydrogen atom, a halogen atom, a methyl group, or a trifluoromethyl group;
X^{A} represents a single bond, a phenylene group, a naphthylene group, or *—C(═O)—O—X^{A1}—, X^{A1} representing a saturated hydrocarbylene group having 1 to 10 carbon atoms, a phenylene group, or a naphthylene group, the saturated hydrocarbylene group optionally containing a hydroxy group, an ether bond, an ester bond, or a lactone ring, and “*” representing an attachment point to the carbon atom of the main chain;
“p” represents 1, 2, 3, or 4;
R³¹ represents a p-valent hydrocarbon group having 1 to 40 carbon atoms or a p-valent heterocyclic group having 2 to 40 carbon atoms, when “p” is 2, the R³¹ optionally being an ether bond, a carbonyl group, an azo group, a thioether bond, a carbonate bond, a carbamate bond, a sulfinyl group, or a sulfonyl group, part of all of hydrogen atoms of the p-valent hydrocarbon group or the p-valent heterocyclic group optionally being substituted with a group containing a heteroatom, and part of —CH₂— of the p-valent hydrocarbon group optionally being substituted with a group containing a heteroatom; and
R³² represents a single bond or a hydrocarbylene group having 1 to 10 carbon atoms, part or all of hydrogen atoms of the hydrocarbylene group optionally being substituted with a group containing a heteroatom, part of —CH₂— of the hydrocarbylene group optionally being substituted with a group containing a heteroatom, and when “p” is 2, 3, or 4, the R³²s being identical to or different from each other.

As the carboxy-group-containing compound contained in the inventive resist composition, such a polymer or monomolecular compound is preferable.

The resist composition preferably further comprises at least one kind of hypervalent iodine compound represented by the following general formula (4) or (5),

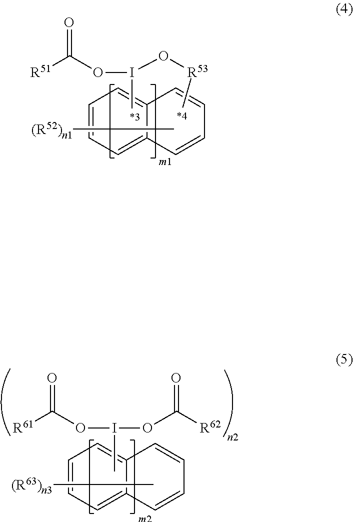

wherein “m1” and “m2” each represent an integer of 0 to 2, “n1” representing an integer of 0 to 4 when “m1” is 0, an integer of 0 to 6 when “m1” is 1, and an integer of 0 to 8 when “m1” is 2, when “m2” is 0, “n2” representing an integer of 1 to 3, “n3” representing an integer of 0 to 5, and 1≤(n2+3)≤6 being satisfied, when “m2” is 1, “n2” representing an integer of 1 to 3, “n3” representing an integer of 0 to 7, and 1≤(n2+n3)≤8 being satisfied, and when “m2” is 2, “n2” representing an integer of 1 to 3, “n3” representing an integer of 0 to 9, and 1≤(n2+n3)≤10 being satisfied; R⁵¹ represents a halogen atom or a hydrocarbyl group having 1 to 10 carbon atoms and optionally containing a heteroatom; R⁵² represents a halogen atom or a hydrocarbyl group having 1 to 40 carbon atoms and optionally containing a heteroatom, when “n1” is 2 to 8, the R⁵²s being identical to or different from each other and the R⁵²s optionally being bonded to each other to form a ring together with the carbon atoms of the aromatic ring bonded to the R⁵²s; R⁵³ represents a hydrocarbylene group having 1 to 10 carbon atoms and optionally containing a heteroatom; “>3” and “*4” each represent an attachment point to one of the carbon atoms of the aromatic ring in the formula, provided that “*3” and “*4” are bonded to adjacent carbon atoms of the aromatic ring; R⁶¹ and R⁶² each independently represent a halogen atom or a hydrocarbyl group having 1 to 10 carbon atoms and optionally containing a heteroatom, the R⁶¹ and the R⁶² optionally being bonded to each other to form a ring together with the carbon atoms bonded thereto and the atoms between the carbon atoms, and when “n2” is 2 or 3, the R⁶¹s and the R⁶²s being identical to or different from each other; and R⁶³ represents a halogen atom or a hydrocarbyl group having 1 to 40 carbon atoms and optionally containing a heteroatom, when “n3” is 2 to 9, the R⁶³s being identical to or different from each other and the R⁶³s optionally being bonded to each other to form a ring together with the carbon atoms of the aromatic ring bonded to the R⁶³s.

As a hypervalent iodine compound further contained in the inventive resist composition, the tricoordinate hypervalent iodine compound represented by the general formula (4) or the general formula (5) is preferable. When such a tricoordinate hypervalent iodine (III) compound, having an aryl group and a carboxylate ligand, is mixed with a carboxy-group-containing compound, the exchange of the compound and the carboxylate ligand occurs easily by an equilibrium reaction in the same manner as the hypervalent iodine compound represented by the general formula (1). In this event, by removing the original carboxylate ligand from the reaction system, equilibrium shifts in a direction to generate a hypervalent iodine compound having a new ligand, and the ligand exchange progresses. Thus, a polymer in which a carboxy-group-containing compound is crosslinked with a hypervalent iodine compound is achieved. When at least one kind of hypervalent iodine compound represented by the general formula (4) or (5) is further contained, polymerization progresses more favorably.

The present invention also provides a laminate comprising: a substrate; and a resist film, obtained from the above-described resist composition, on the substrate.

In a laminate including a resist film obtained from the inventive resist composition, the resist film, which is a film body of the above-described resist composition, has high sensitivity, also exhibits excellent limiting resolution, is effective for precise fine processing, and in addition, can be applied to either positive or negative patterning. Therefore, the laminate has a wide range of uses, and is highly useful in resist process technology.

In this case, the laminate can further comprise a resist underlayer film between the substrate and the resist film.

The inventive laminate can be as described above in accordance with requirements.

The resist film preferably contains a product made by a ligand exchange reaction of the hypervalent iodine compound represented by the general formula (1) and the carboxy-group-containing compound.

When the product made by the ligand exchange reaction is contained, a polymer in which the carboxy-group-containing compound is crosslinked with the hypervalent iodine compound is achieved.

The present invention also provides a patterning process comprising the steps of:
forming a resist film by using the above-described resist composition on a substrate or on a resist underlayer film of a substrate on which the resist underlayer film has been laminated;
exposing the resist film by using a high-energy beam; and
developing the exposed resist film by using a developer.

In the inventive patterning process, a resist composition that is excellent in sensitivity and resolution in photolithography using a high-energy beam, especially electron beam (EB) lithography and EUV lithography, is used, and therefore, the patterning process is useful for finer patterning.

In this case, the high-energy beam is preferably an i-line, a KrF excimer laser beam, an ArF excimer laser beam, an electron beam, or an extreme ultraviolet ray.

In the inventive patterning process, using such a high-energy beam makes finer patterning possible.

In the inventive patterning process, the developer may dissolve exposed portions and not dissolve unexposed portions.

According to the inventive patterning process, a positive pattern can be formed by selecting the developer appropriately, and therefore, the patterning process can be widely applied to various kinds of fine patterning.

In the inventive patterning process, the developer may dissolve unexposed portions and not dissolve exposed portions.

According to the inventive patterning process, a negative pattern can be formed by selecting the developer appropriately, and therefore, the patterning process can be widely applied to various kinds of fine patterning.

### Advantageous Effects of Invention

The inventive resist composition containing, as a main component, a particular hypervalent iodine compound having an SF₅ group can achieve both high sensitivity and high resolution, particularly in EB lithography and EUV lithography, and is extremely useful on forming a fine pattern.

### DESCRIPTION OF EMBODIMENTS

As stated above, there have been demands for the development of a non-chemically amplified resist composition excellent in sensitivity and resolution in high-energy beam, especially electron beam (EB) lithography and EUV lithography.

To achieve the object, the present inventors have studied earnestly and found out that a resist composition containing, as a main component, a particular hypervalent iodine compound having an SF₅ group allows favorable solubility in the entire resist, has extremely high sensitivity and can give a resist film that exhibits excellent resolution in the above-described high-energy beam, especially electron beam (EB) lithography and EUV lithography, and is extremely effective for precise fine processing. Thus, the present invention has been achieved.

That is, the present invention is a hypervalent iodine compound having a structure represented by the following general formula (1),

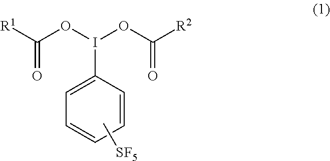

wherein R¹ and R² each independently represent a halogen atom or a hydrocarbyl group having 1 to 10 carbon atoms and optionally containing a heteroatom, the R¹ and the R² optionally being bonded to each other to form a ring together with the carbon atoms bonded thereto and the atoms between the carbon atoms.

Hereinafter, the present invention will be described in detail, but the present invention is not limited thereto. Note that, in the present description, the recitations of numerical ranges by endpoints include all numbers subsumed within that range (e. g. “0 to 3” includes 0, 1, 2, and 3).

### [Resist Composition]

The inventive resist composition mainly contains a predetermined hypervalent iodine compound, a carboxy-group-containing compound, and a solvent. Incidentally, a carboxy group is an atomic group (functional group) having the structure “—C(═O)OH”.

### [Hypervalent Iodine Compound]

The hypervalent iodine compound essential in the present invention is represented by the following general formula (1).

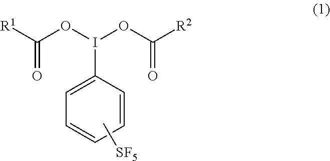

In the formula, R¹ and R² each independently represent a halogen atom or a hydrocarbyl group having 1 to 10 carbon atoms and optionally containing a heteroatom, the R¹ and the R² optionally being bonded to each other to form a ring together with the carbon atoms bonded thereto and the atoms between the carbon atoms.

In the general formula (1), R¹ and R² each represent a halogen atom or a hydrocarbyl group having 1 to 10 carbon atoms and optionally containing a heteroatom. Specific examples of the halogen atom include a fluorine atom, a chlorine atom, a bromine atom, and an iodine atom. The hydrocarbyl group having 1 to 10 carbon atoms may be saturated or unsaturated, and may be linear, branched, or cyclic. Specific examples thereof include: alkyl groups having 1 to 10 carbon atoms, such as a methyl group, an ethyl group, an n-propyl group, an isopropyl group, an n-butyl group, an isobutyl group, a sec-butyl group, a tert-butyl group, an n-pentyl group, a tert-pentyl group, an n-hexyl group, an n-octyl group, a 2-ethylhexyl group, an n-nonyl group, and an n-decyl group; cyclic saturated hydrocarbyl groups having 3 to 10 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.0^{2,6}]decanyl group, and an adamantyl group; alkenyl groups having 2 to 10 carbon atoms, such as a vinyl group and an allyl group; aryl groups having 6 to 10 carbon atoms, such as a phenyl group and a naphthyl group; and groups which are combinations of these groups. Furthermore, part or all of the hydrogen atoms of the hydrocarbyl group may be substituted with a group containing a heteroatom, such as an oxygen atom, a sulfur atom, a nitrogen atom, or a halogen atom, and part of the —CH₂— of the hydrocarbyl group may be substituted with a group containing a heteroatom such as an oxygen atom, a sulfur atom, or a nitrogen atom. The resulting hydrocarbyl group may contain a hydroxy group, a cyano group, a halogen atom, a carbonyl group, an ether bond, a thioether bond, an ester bond, a sulfonic acid ester bond, a carbonate bond, a carbamate bond, a lactone ring, a sultone ring, a carboxylic anhydride (—C(═O)—O—C(═O)—), etc. As the R¹ and the R², a hydrocarbyl group having 1 to 4 carbon atoms or a fluorinated hydrocarbyl group having 1 to 4 carbon atoms is preferable, and a hydrocarbyl group having 1 to 4 carbon atoms is more preferable.

Specific examples of the hypervalent iodine compound represented by the general formula (1) include the following, but are not limited thereto. Note that, in the following formulae, Me represents a methyl group.

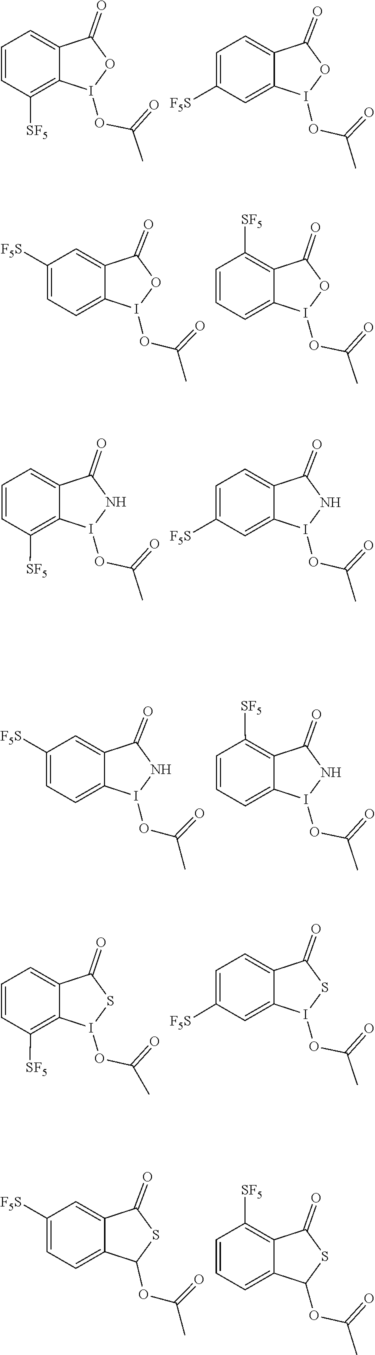

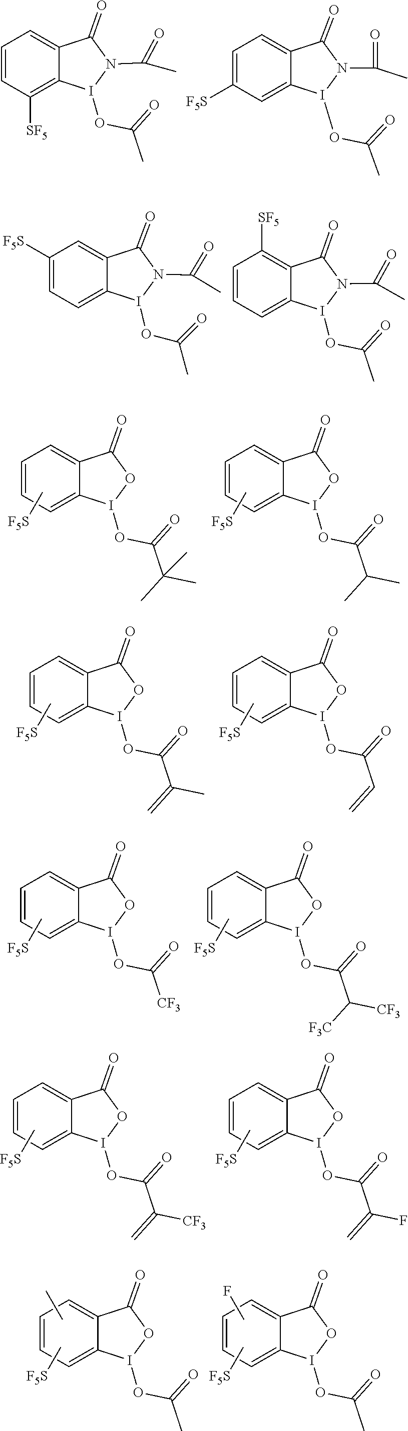

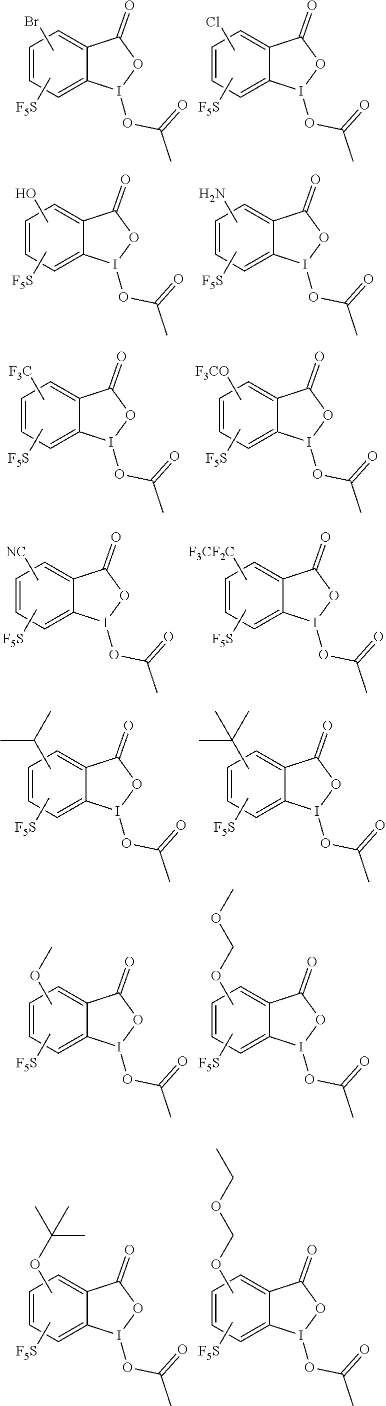

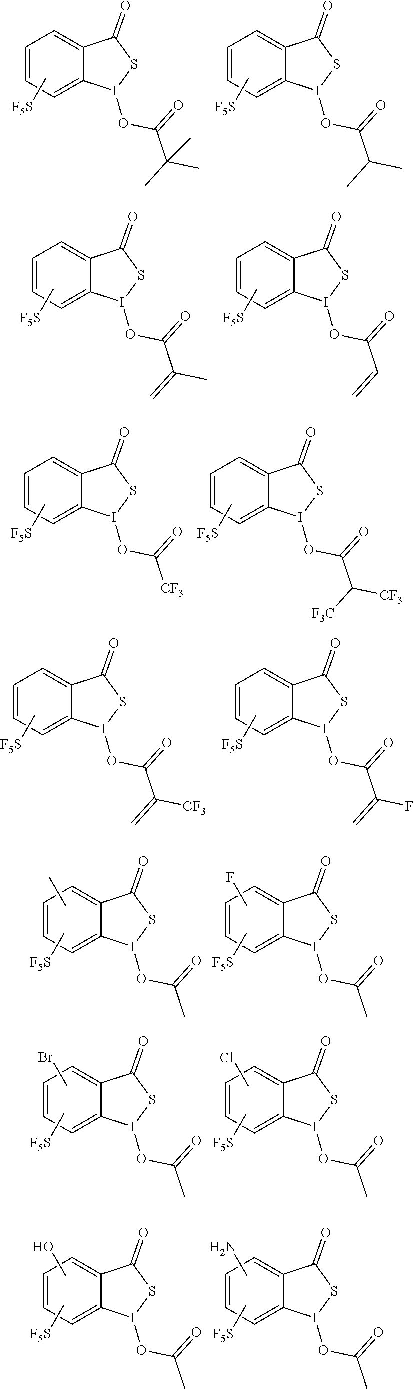

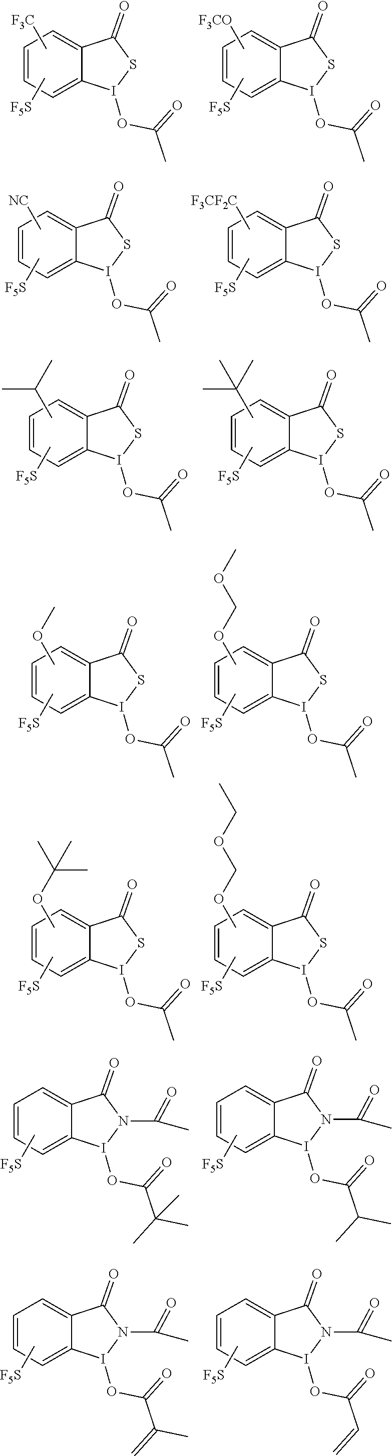

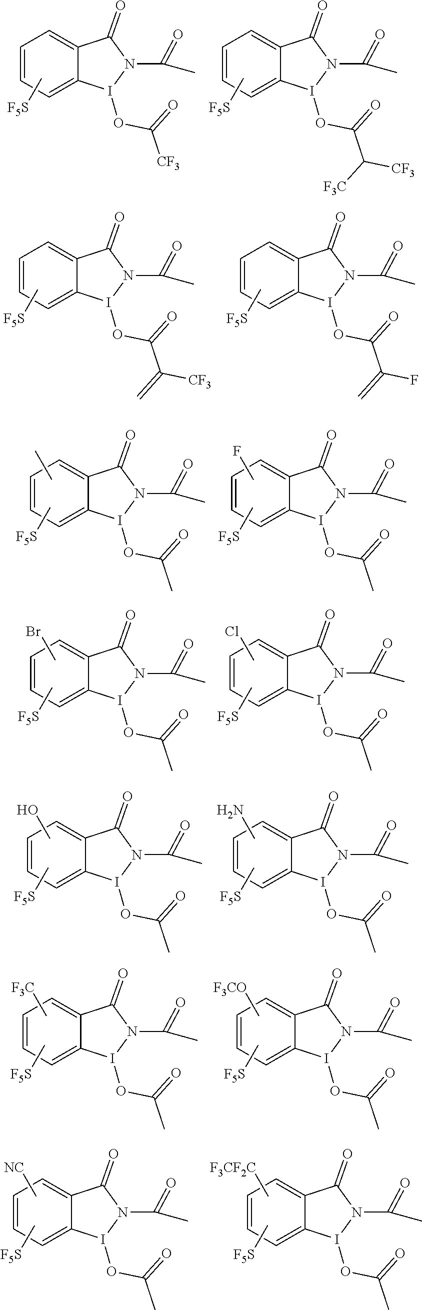

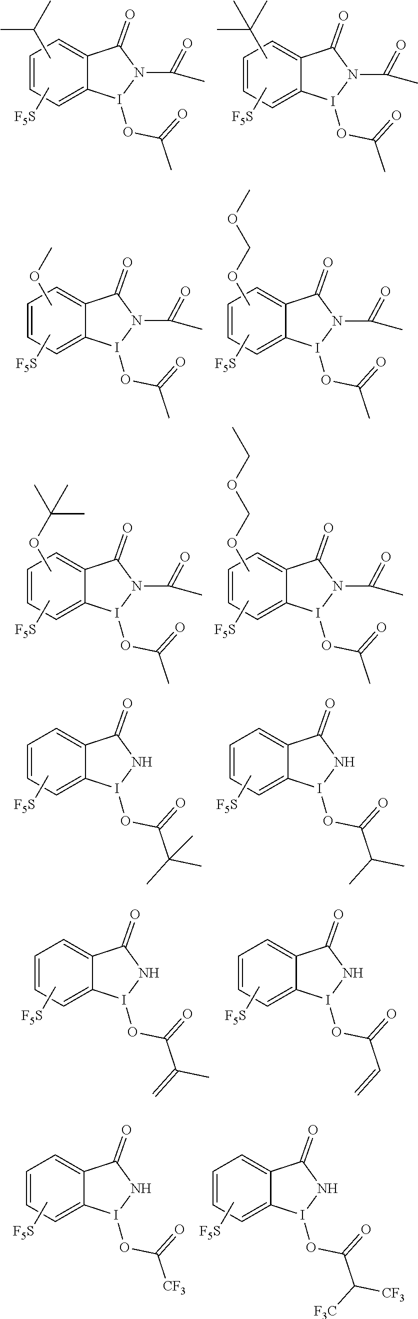

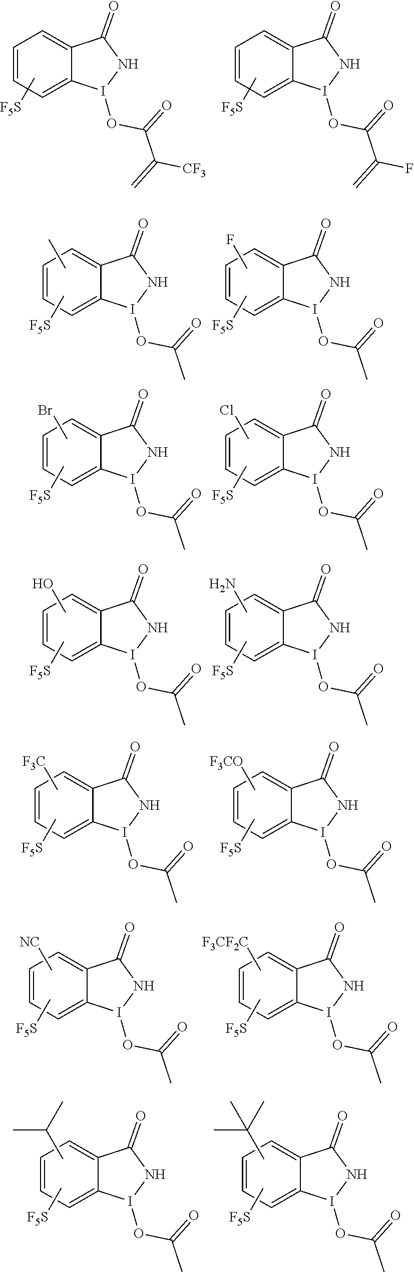

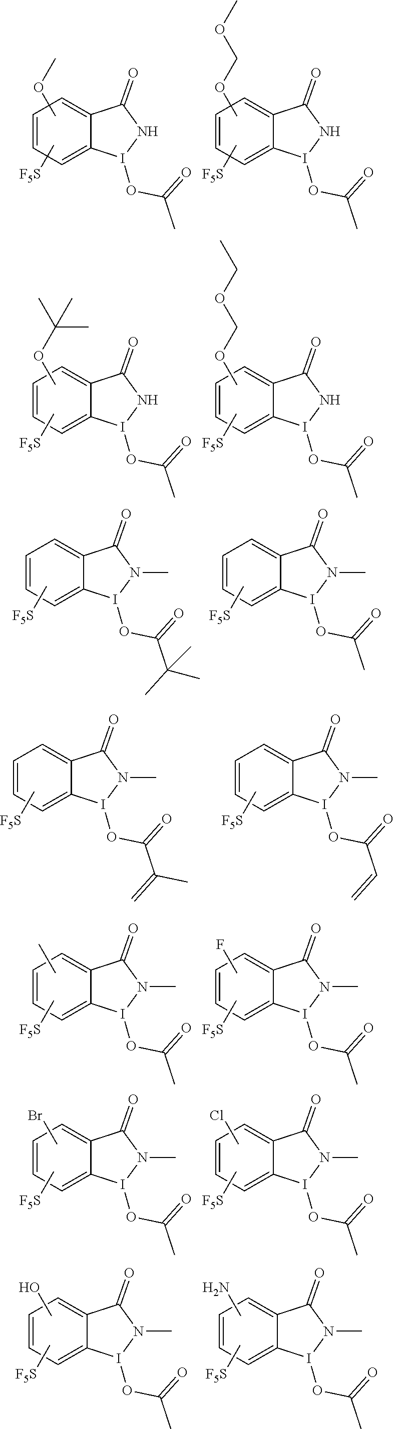

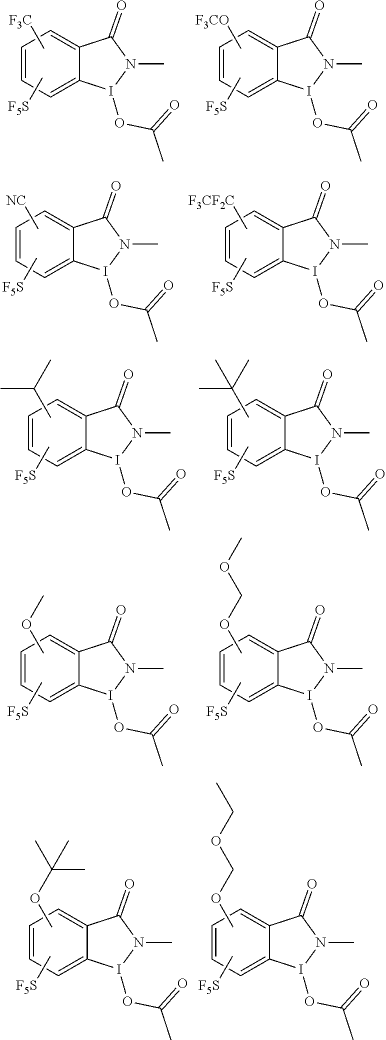

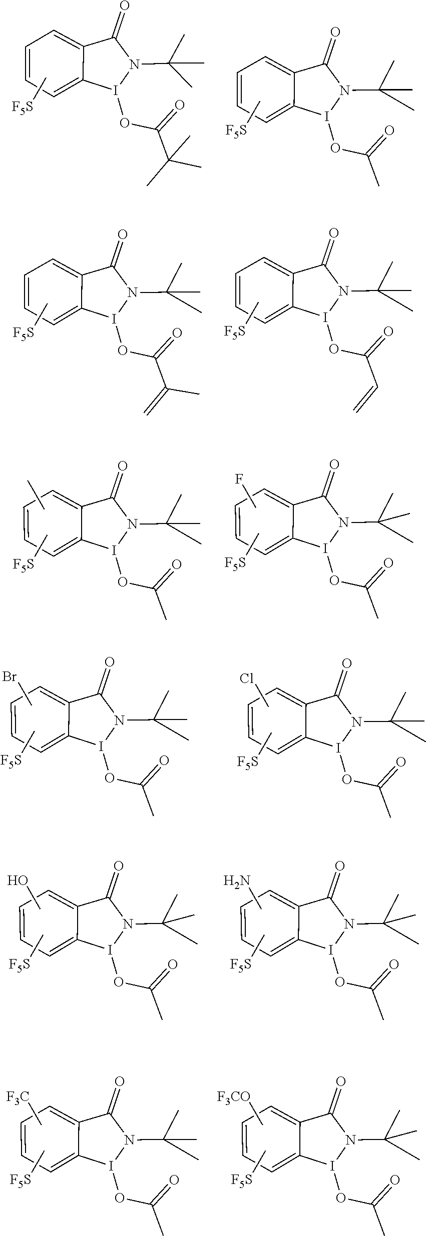

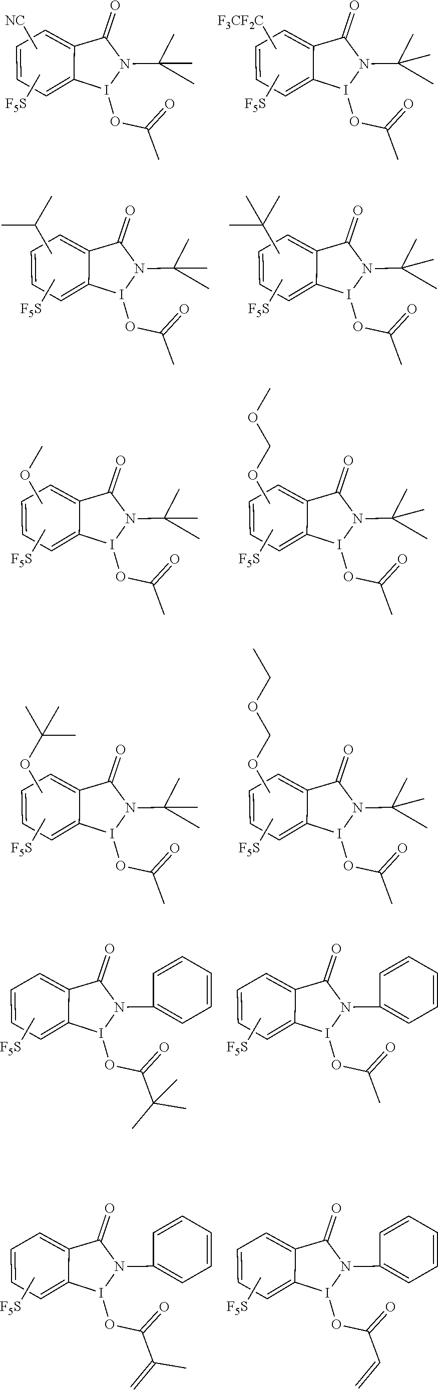

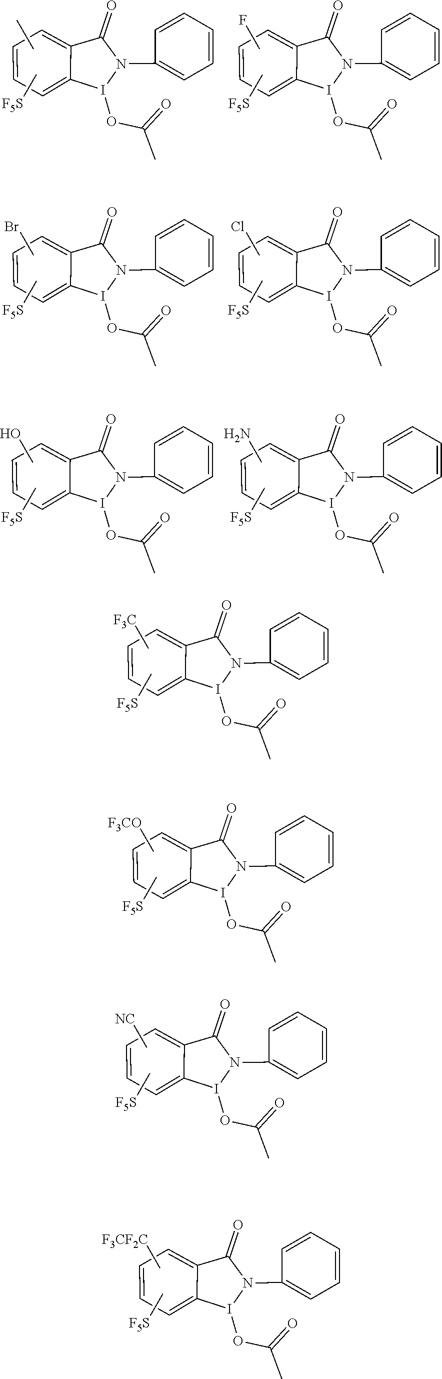

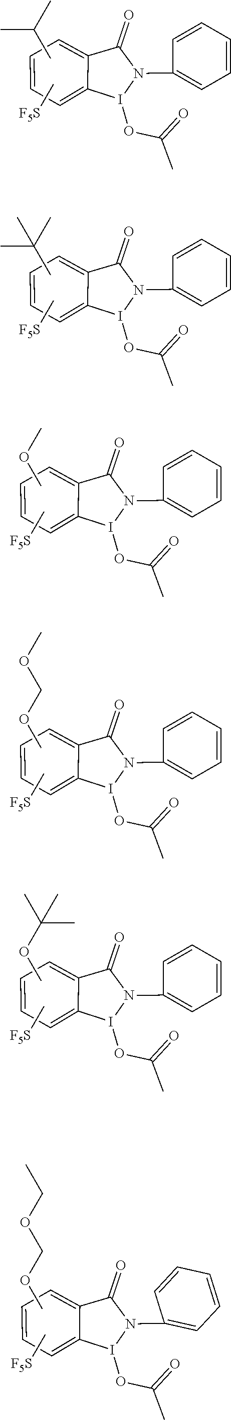

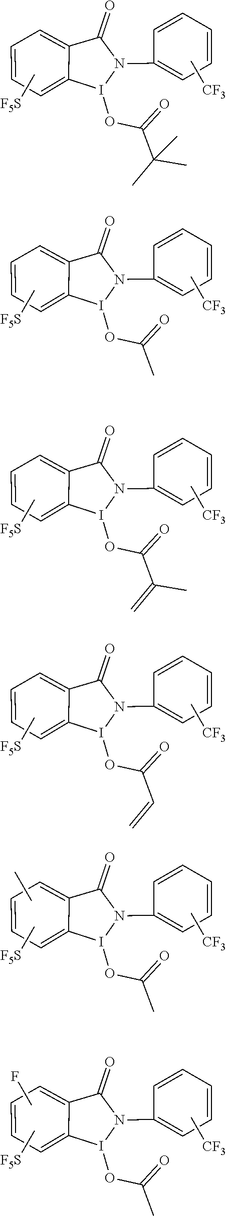

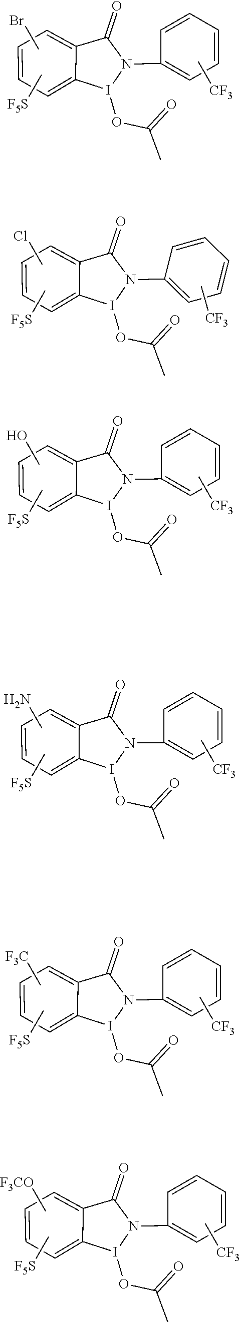

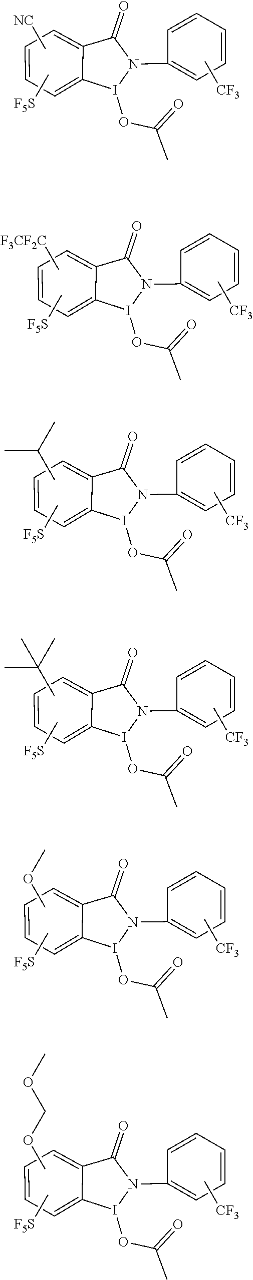

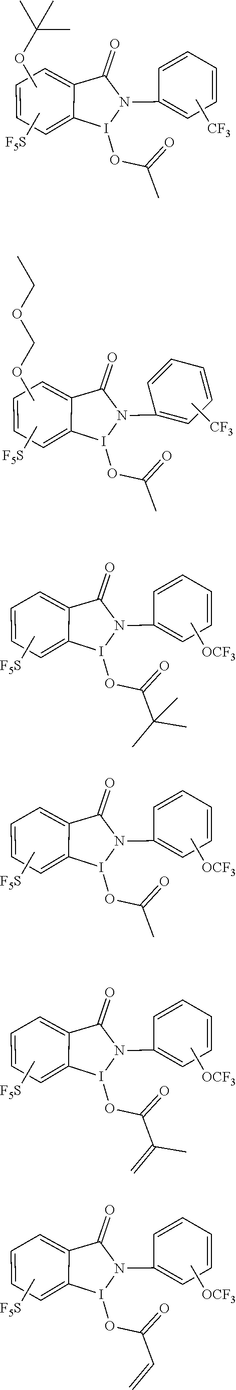

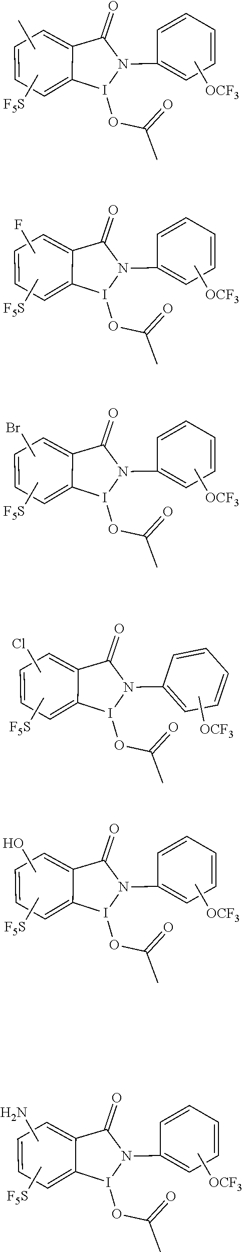

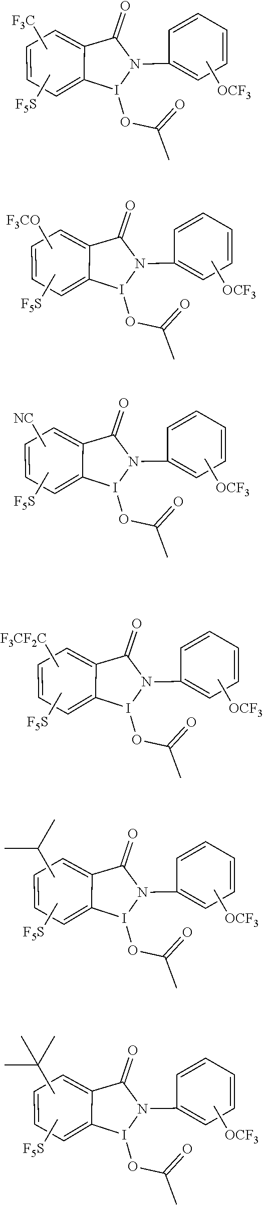

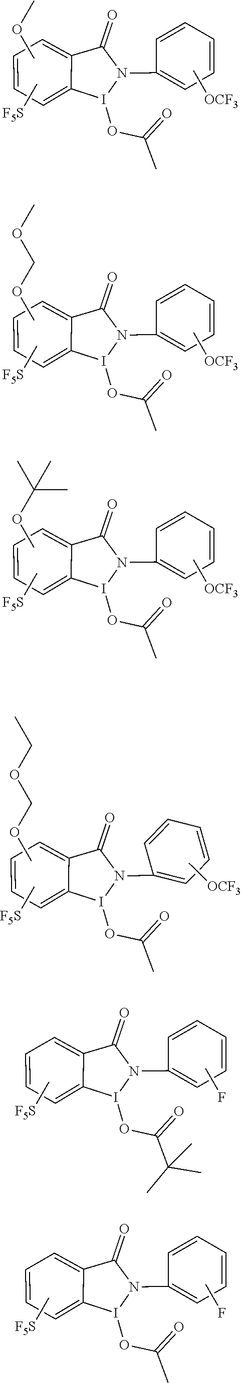

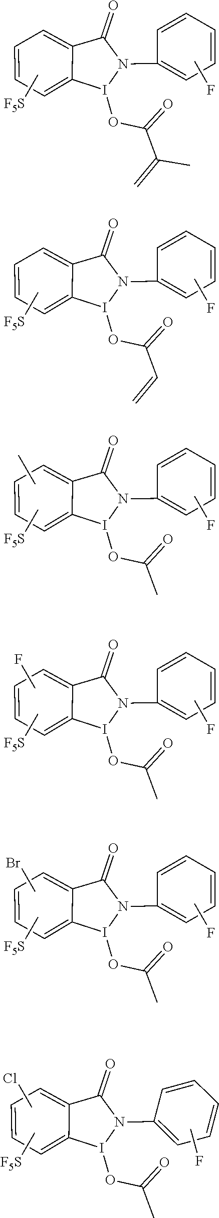

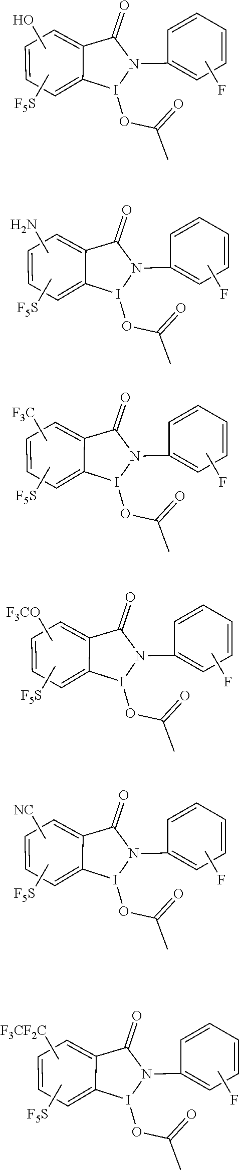

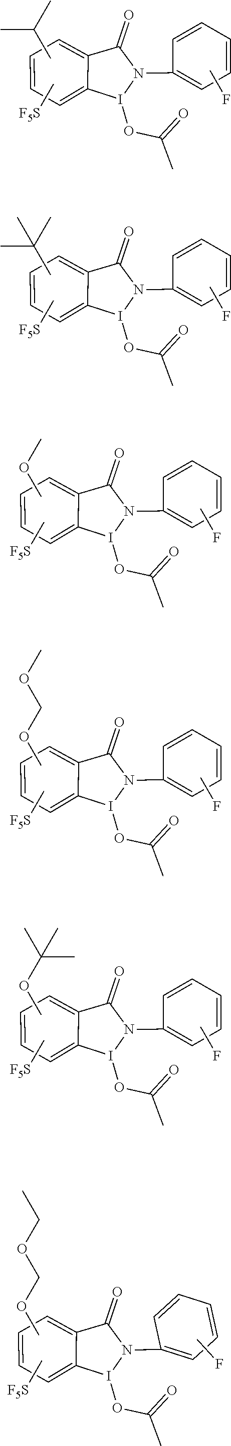

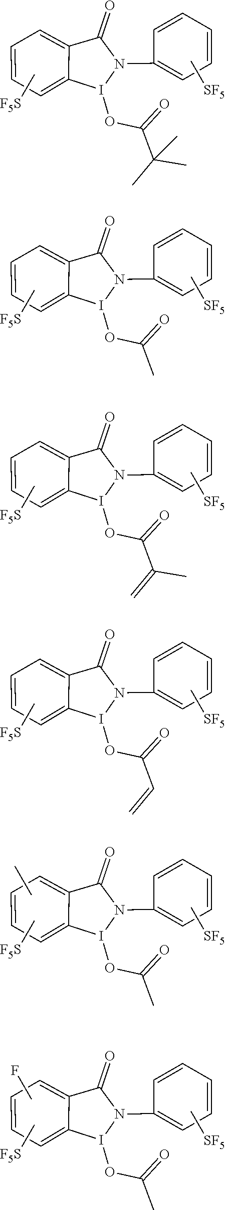

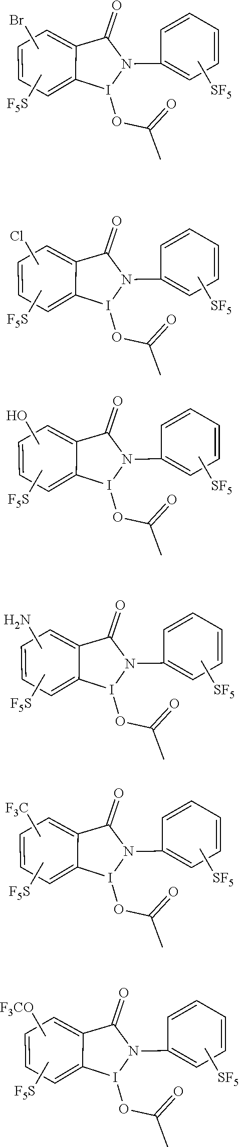

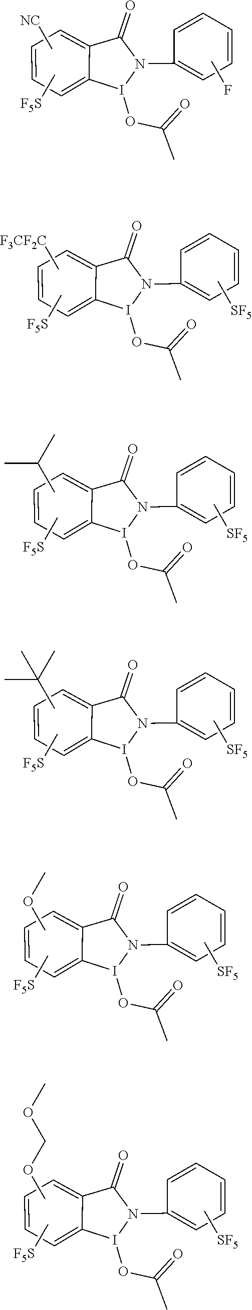

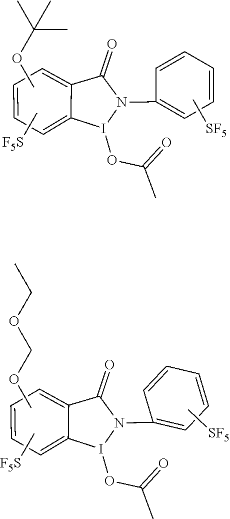

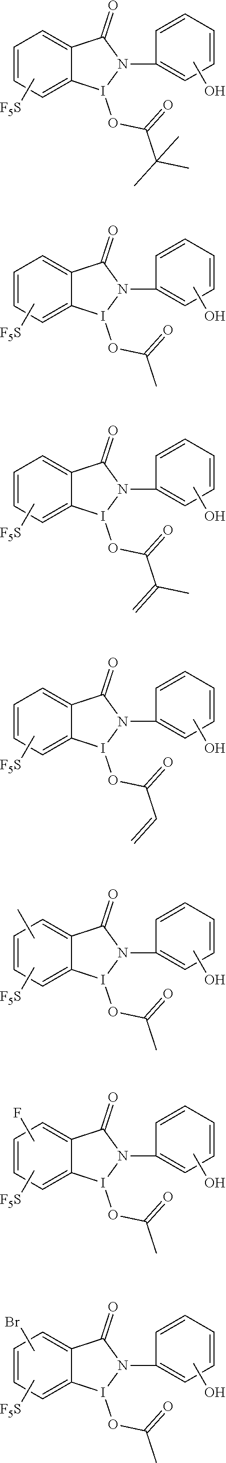

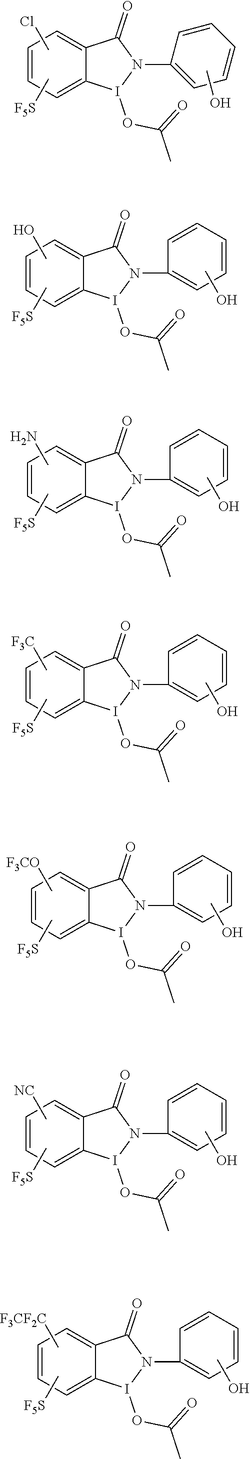

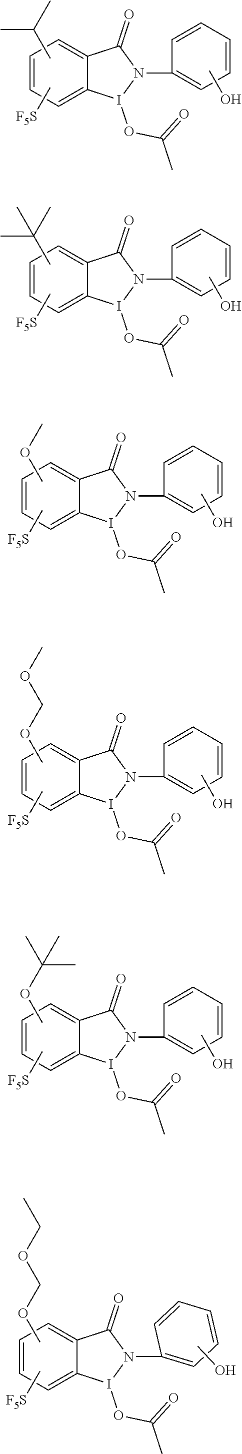

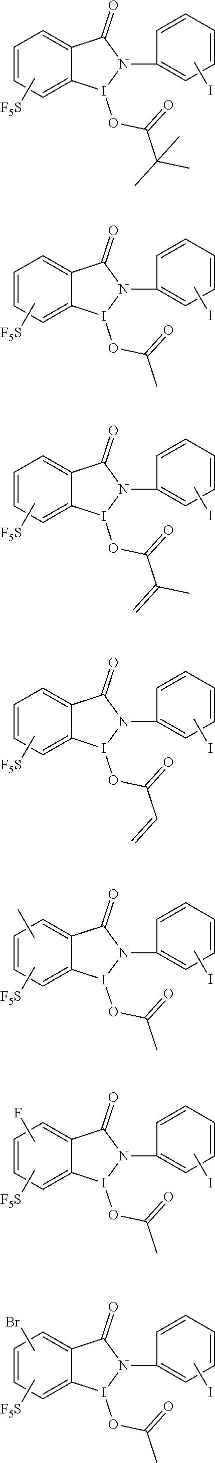

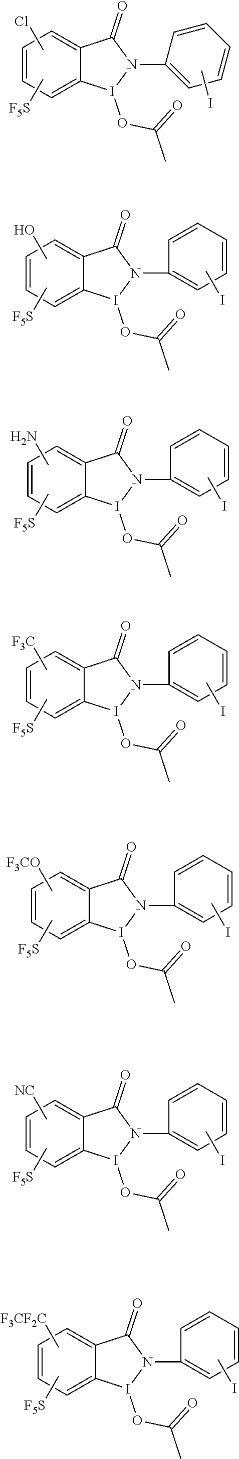

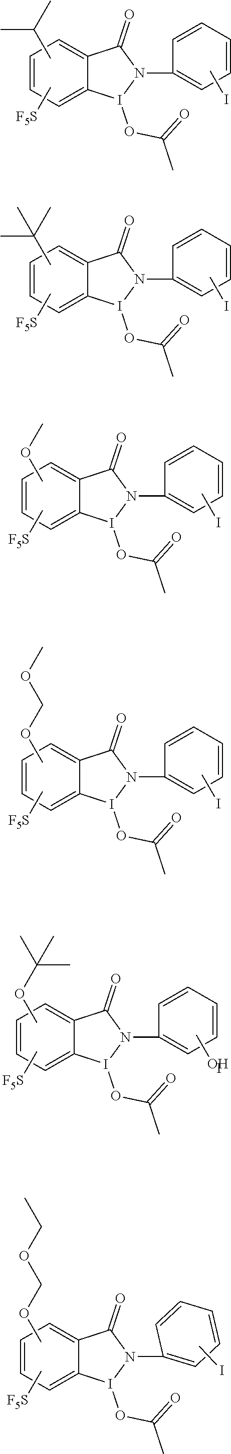

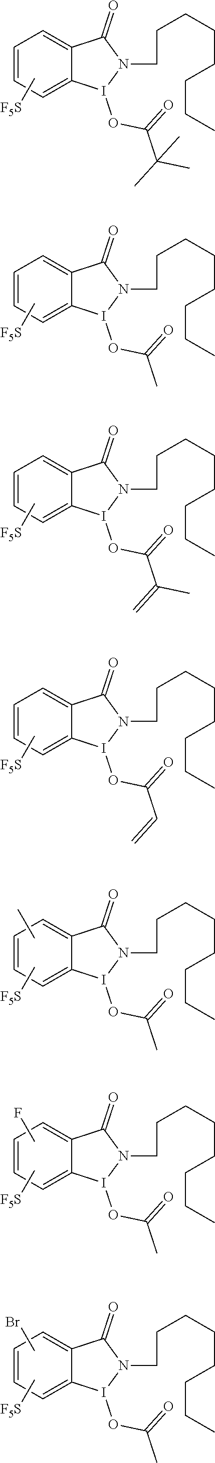

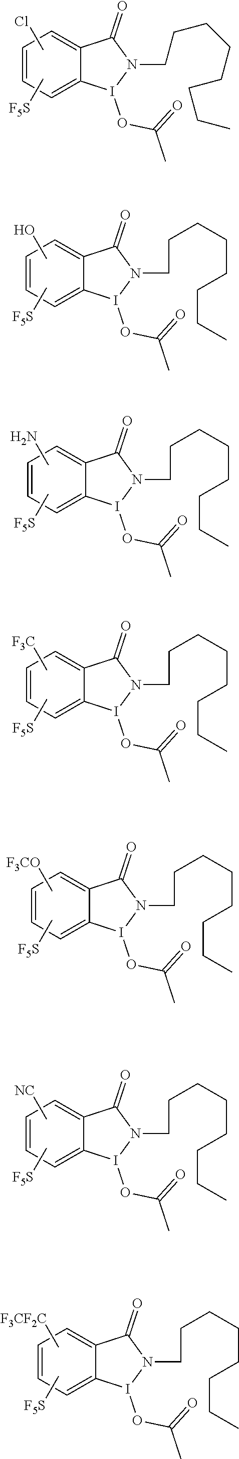

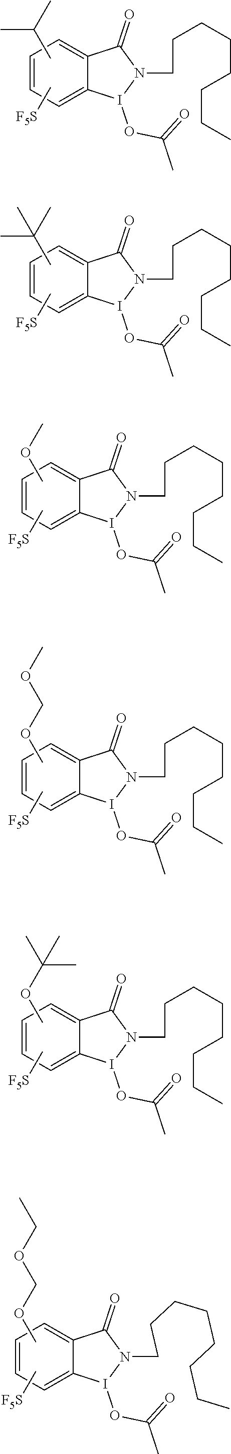

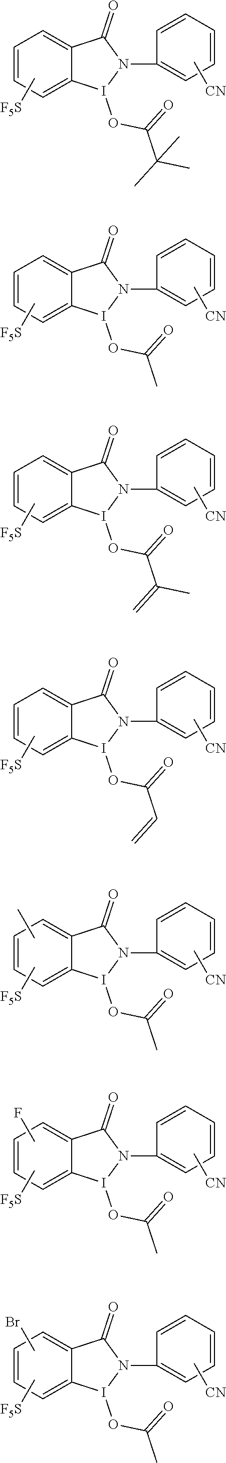

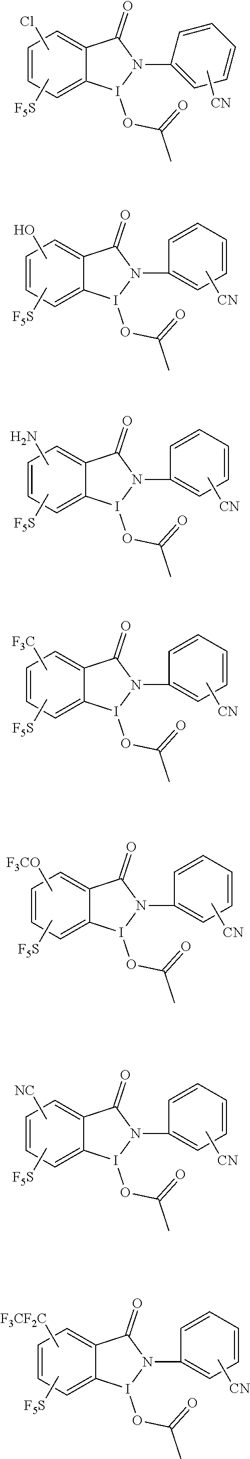

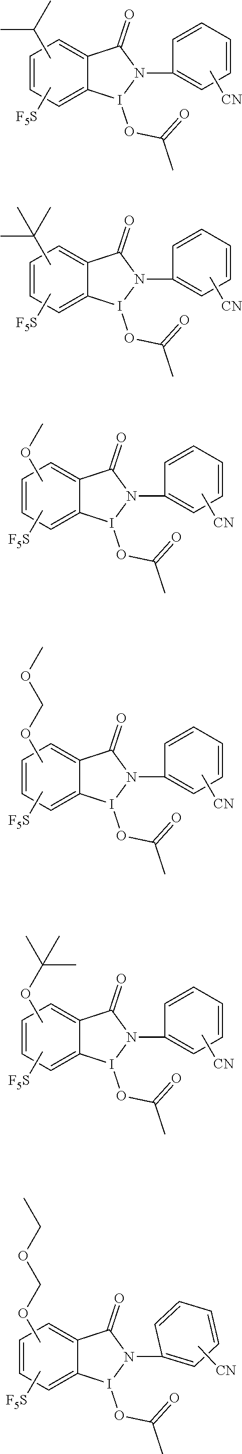

A resist composition containing, as a main component, the inventive hypervalent iodine compound, having an SF₅ group, has improved solubility in a solvent, and therefore, is favorable. Furthermore, a resist composition mainly containing the inventive hypervalent iodine compound has enhanced resolution in photolithography using a high-energy beam, especially EB lithography and EUV lithography, and can be used in a patterning process excellent in roughness resolution.

### [Method for Manufacturing Hypervalent Iodine Compound]

The hypervalent iodine compound represented by the general formula (1) used in the present invention can be obtained by a known method. For example, when the target hypervalent iodine compound contains iodine (III) and SF₅, the compound can be obtained by subjecting SF₅-containing iodobenzamide to oxidization and acetylation with an oxidizing agent such as peracetic acid. Regarding the synthesizing method, JP2013-119541A etc. can be consulted, for example.

### [Carboxy-Group-Containing Compound]

The carboxy-group-containing compound is preferably a polymer including a repeating unit represented by the following general formula (2) and/or a compound represented by the following general formula (3).

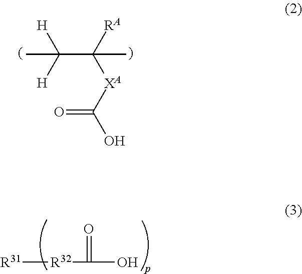

In the formulae, R^{A} represents a hydrogen atom, a halogen atom, a methyl group, or a trifluoromethyl group;
X^{A} represents a single bond, a phenylene group, a naphthylene group, or *—C(═O)—O—X^{A1}—, X^{A1} representing a saturated hydrocarbylene group having 1 to 10 carbon atoms, a phenylene group, or a naphthylene group, the saturated hydrocarbylene group optionally containing a hydroxy group, an ether bond, an ester bond, or a lactone ring, and “*” representing an attachment point to the carbon atom of the main chain;
“p” represents 1, 2, 3, or 4;
R³¹ represents a p-valent hydrocarbon group having 1 to 40 carbon atoms or a p-valent heterocyclic group having 2 to 40 carbon atoms, when “p” is 2, the R³¹ optionally being an ether bond, a carbonyl group, an azo group, a thioether bond, a carbonate bond, a carbamate bond, a sulfinyl group, or a sulfonyl group, part or all of hydrogen atoms of the p-valent hydrocarbon group or the p-valent heterocyclic group optionally being substituted with a group containing a heteroatom, and part of —CH₂— of the p-valent hydrocarbon group optionally being substituted with a group containing a heteroatom; and
R³² represents a single bond or a hydrocarbylene group having 1 to 10 carbon atoms, part or all of hydrogen atoms of the hydrocarbylene group optionally being substituted with a group containing a heteroatom, part of —CH₂— of the hydrocarbylene group optionally being substituted with a group containing a heteroatom, and when “p” is 2, 3, or 4, the R³²s being identical to or different from each other.

In the general formula (2), R^{A} represents a hydrogen atom, a halogen atom, a methyl group, or a trifluoromethyl group; and X^{A} represents a single bond, a phenylene group, a naphthylene group, or *—C(═O)—O—X^{A1}—, X^{A1} representing a saturated hydrocarbylene group having 1 to 10 carbon atoms, a phenylene group, or a naphthylene group, the saturated hydrocarbylene group optionally containing a hydroxy group, an ether bond, an ester bond, or a lactone ring, and “*” representing an attachment point to the carbon atom of the main chain.

In the general formula (3), “p” represents 1, 2, 3, or 4.

In the general formula (3), R³¹ represents a p-valent hydrocarbon group having 1 to 40 carbon atoms or a p-valent heterocyclic group having 2 to 40 carbon atoms, when “p” is 2, the R³¹ optionally being an ether bond, a carbonyl group, an azo group, a thioether bond, a carbonate bond, a carbamate bond, a sulfinyl group, or a sulfonyl group, part or all of hydrogen atoms of the p-valent hydrocarbon group or the p-valent heterocyclic group optionally being substituted with a group containing a heteroatom, and part of —CH₂— of the p-valent hydrocarbon group optionally being substituted with a group containing a heteroatom.

In the general formula (3), R³² represents a single bond or a hydrocarbylene group having 1 to 10 carbon atoms, part or all of hydrogen atoms of the hydrocarbylene group optionally being substituted with a group containing a heteroatom, part of —CH₂— of the hydrocarbylene group optionally being substituted with a group containing a heteroatom, and when “p” is 2, 3, or 4, the R³²s being identical to or different from each other.

In the general formula (3), the p-valent hydrocarbon group represented by R³¹ may be saturated or unsaturated, and may be linear, branched, or cyclic. The p-valent hydrocarbon group is a group obtained by “p” hydrogen atoms being removed from a hydrocarbon. Examples of the hydrocarbon include alkanes having 1 to 40 carbon atoms, alkenes having 2 to 40 carbon atoms, alkynes having 2 to 40 carbon atoms, cyclic saturated hydrocarbons having 3 to 40 carbon atoms, cyclic unsaturated hydrocarbons having 3 to 40 carbon atoms, and aromatic hydrocarbons having 6 to 40 carbon atoms.

Regarding the p-valent hydrocarbon group represented by R³¹, examples of the alkanes having 1 to 40 carbon atoms include methane, ethane, propane, butane, pentane, hexane, heptane, octane, nonane, decane, and structural isomers thereof.

Regarding the p-valent hydrocarbon group represented by R³¹, examples of the alkenes having 2 to 40 carbon atoms include ethylene, propylene, butene, pentene, hexene, heptene, octene, nonene, decene, and structural isomers thereof.

Regarding the p-valent hydrocarbon group represented by R³¹, examples of the alkynes having 2 to 40 carbon atoms include acetylene, propyne, butyne, pentyne, hexyne, heptyne, octyne, nonyne, decyne, and structural isomers thereof.

Regarding the p-valent hydrocarbon group represented by R³¹, examples of the cyclic saturated hydrocarbons having 3 to 40 carbon atoms include cyclopropane, cyclobutane, cyclohexane, cycloheptane, cyclooctane, adamantane, and norbornane.

Regarding the p-valent hydrocarbon group represented by R³¹, examples of the cyclic unsaturated hydrocarbons having 3 to 40 carbon atoms include cyclopropene, cyclobutene, cyclopentene, cyclohexene, cycloheptene, cyclooctene, and norbornene.

Regarding the p-valent hydrocarbon group represented by R³¹, examples of the aromatic hydrocarbons having 6 to 40 carbon atoms include benzene, naphthalene, and biphenyl.

The p-valent heterocyclic group represented by R³¹ is a group obtained by removing “p” hydrogen atoms from a heterocyclic compound. Examples of the heterocyclic compound include furan, pyridine, pyrazole, and thiazolidine.

Part or all of the hydrogen atoms of the p-valent hydrocarbon group or the p-valent heterocyclic group represented by R³¹ may be substituted with a group containing a heteroatom such as an oxygen atom, a sulfur atom, a nitrogen atom, or a halogen atom. The resulting p-valent hydrocarbon group or p-valent heterocyclic group may contain a hydroxy group, a cyano group, a fluorine atom, a chlorine atom, a bromine atom, an iodine atom, etc. Furthermore, part of the —CH₂— constituting the p-valent hydrocarbon group may be substituted with a group containing a heteroatom such as an oxygen atom, a sulfur atom, or a nitrogen atom. The resulting p-valent hydrocarbon group may contain a carbonyl group, an ether bond, a thioether bond, an ester bond, a sulfonic acid ester bond, a carbonate bond, a carbamate bond, a lactone ring, a sultone ring, a carboxylic anhydride (—C(═O)—O—C(═O)—), etc.

In the general formula (3), the hydrocarbylene group represented by R³² may be saturated or unsaturated, and may be linear, branched, or cyclic. Specific examples thereof include: alkanediyl groups having 1 to 20 carbon atoms, such as a methanediyl group, an ethane-1,1-diyl group, an ethane-1,2-diyl group, a propane-1,2-diyl group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, and a dodecane-1,12-diyl group; cyclic saturated hydrocarbylene groups having 3 to 20 carbon atoms, such as a cyclopentanediyl group, a cyclohexanediyl group, a norbornanediyl group, and an adamantanediyl group; unsaturated aliphatic hydrocarbylene groups having 2 to 20 carbon atoms, such as a vinylene group and a propene-1,3-diyl group; arylene groups having 6 to 20 carbon atoms, such as a phenylene group and a naphthylene group; and groups obtained by combining these groups. Furthermore, part or all of the hydrogen atoms of the hydrocarbylene group may be substituted with a group containing a heteroatom, such as an oxygen atom, a sulfur atom, a nitrogen atom, or a halogen atom, and part of the —CH₂— constituting the hydrocarbylene group may be substituted with a group containing a heteroatom such as an oxygen atom, a sulfur atom, or a nitrogen atom. The resulting hydrocarbylene group may contain a hydroxy group, a cyano group, a fluorine atom, a chlorine atom, a bromine atom, an iodine atom, a carbonyl group, an ether bond, a thioether bond, an ester bond, a sulfonic acid ester bond, a carbonate bond, a carbamate bond, a lactone ring, a sultone ring, a carboxylic anhydride, etc.

Among compounds represented by the general formula (3), compounds in which “p” is 2, 3, or 4 are preferable. In this case, a strong resist film having a high molecular weight can be easily formed when the compound is mixed with the hypervalent iodine compound, and therefore, such compounds are preferable from the viewpoints of etching resistance and developer resistance.

Specific examples of the repeating unit represented by the general formula (2) include the following, but are not limited thereto. Note that, in the following formulae, R^{A} is as defined above.

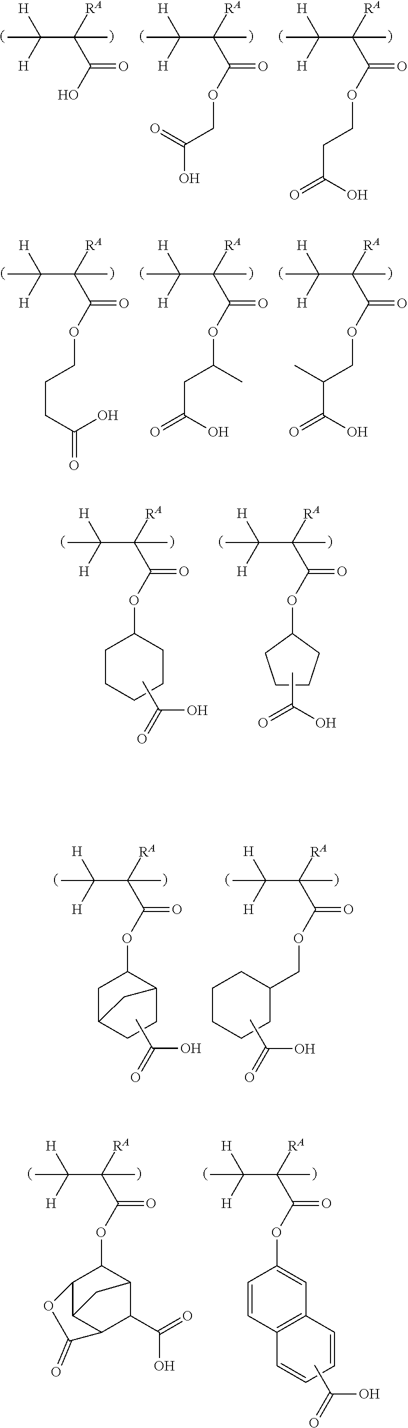

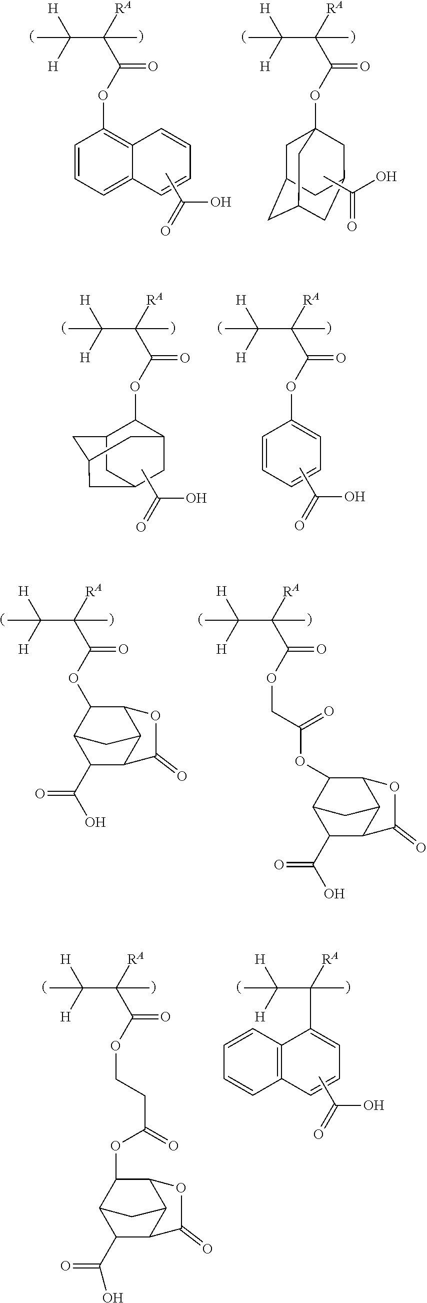

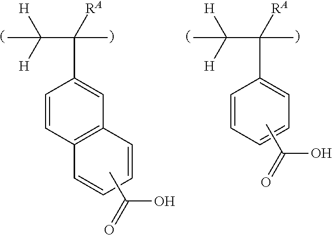

Examples of the compound represented by the general formula (3) include the following, but are not limited thereto. The compound represented by the general formula (3) may be a commercially available product or a synthesized compound.

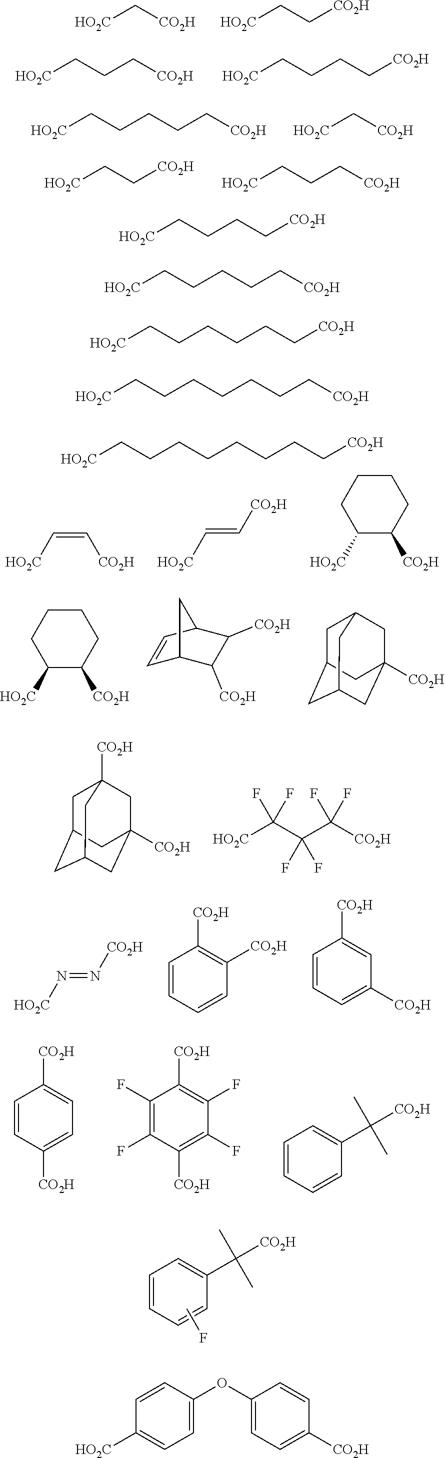

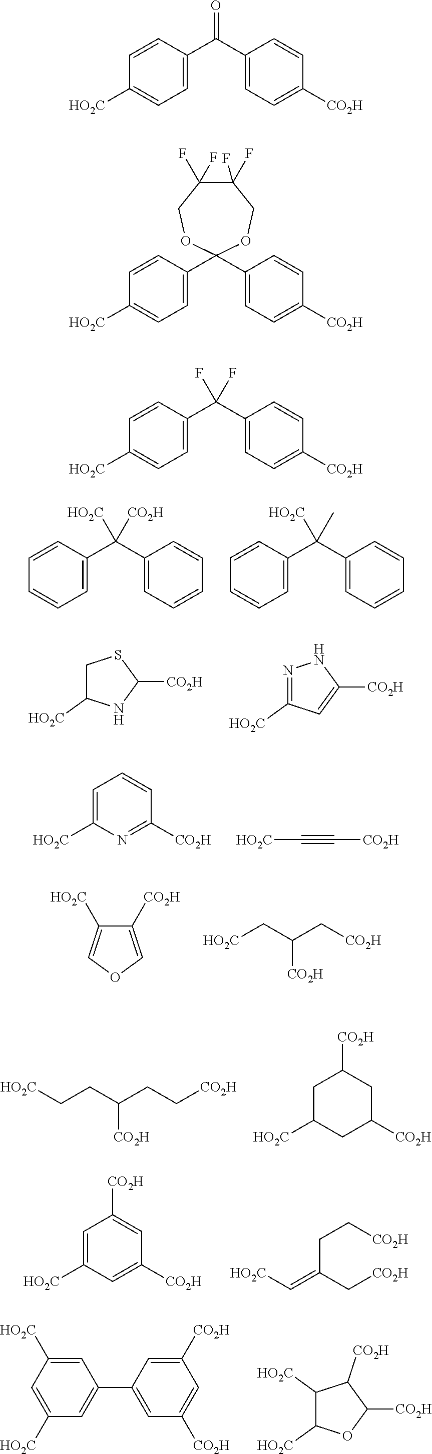

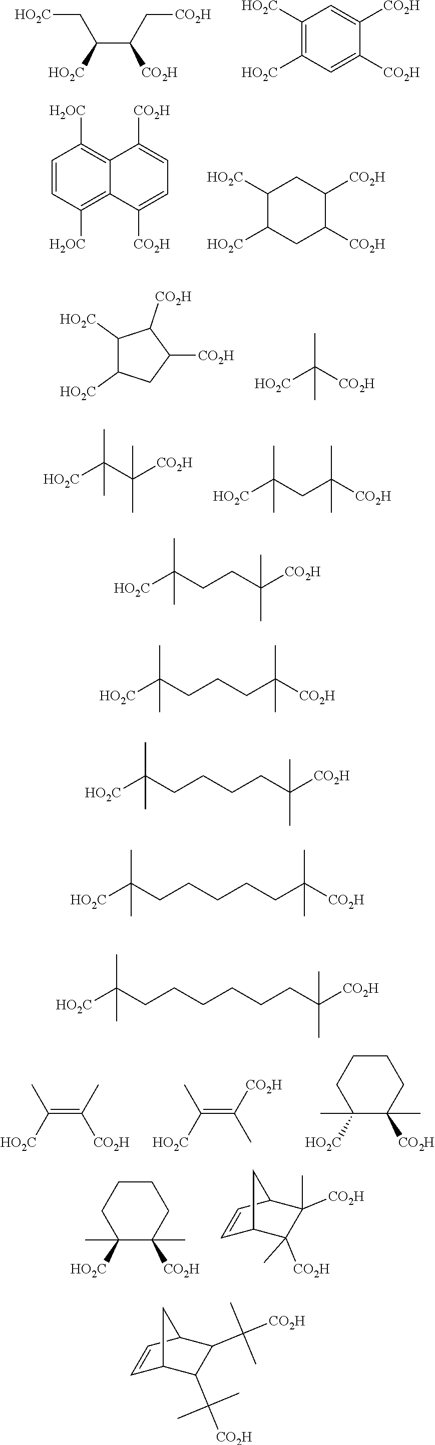

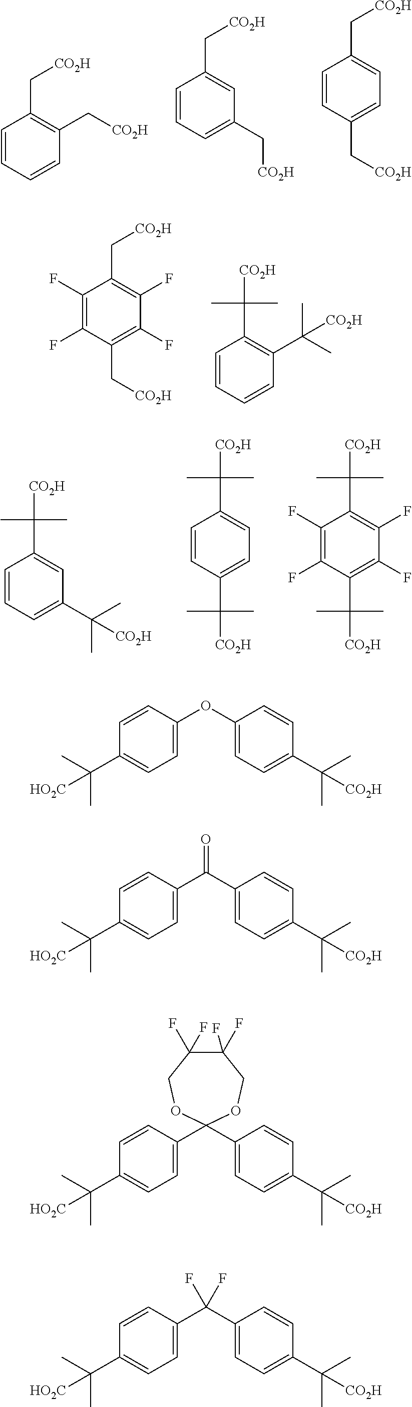

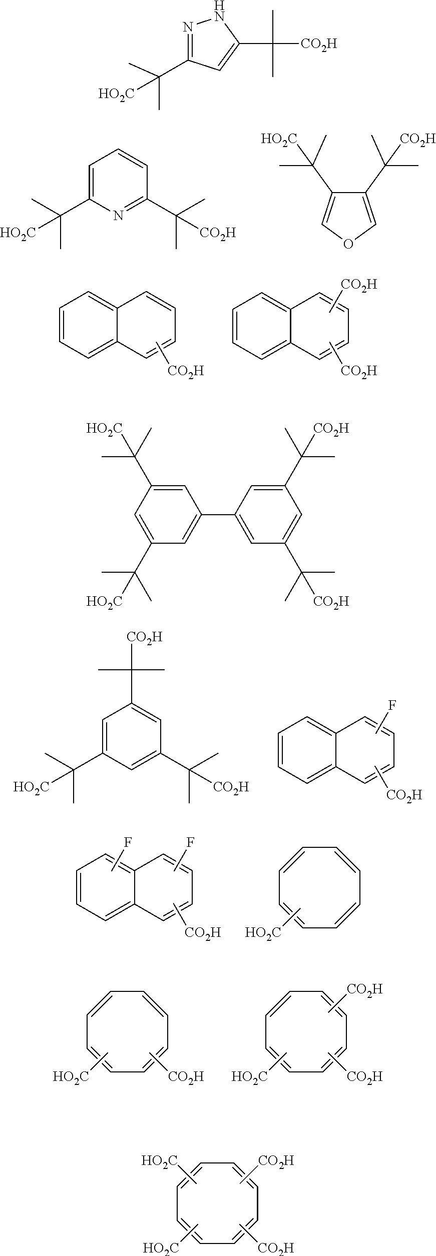

The polymer including the repeating unit represented by the general formula (2) may further include other repeating units (hereinafter, also referred to as “other repeating units”). The other repeating units are not particularly limited, but those which may enhance the solubility of the polymer in a solvent are preferable, because of the polymer being hardly soluble when having only a repeating unit having a carboxy group. As the other repeating units, repeating units having a cyclic structure and repeating units including a styrene skeleton, the units having a rigid skeleton being expected to have high etching resistance, are preferable.

Specific examples of the other repeating units include the following, but are not limited thereto. Note that, in the following formulae, R^{A} is as defined above and each X^{B} independently represents —CH₂— or —O—.

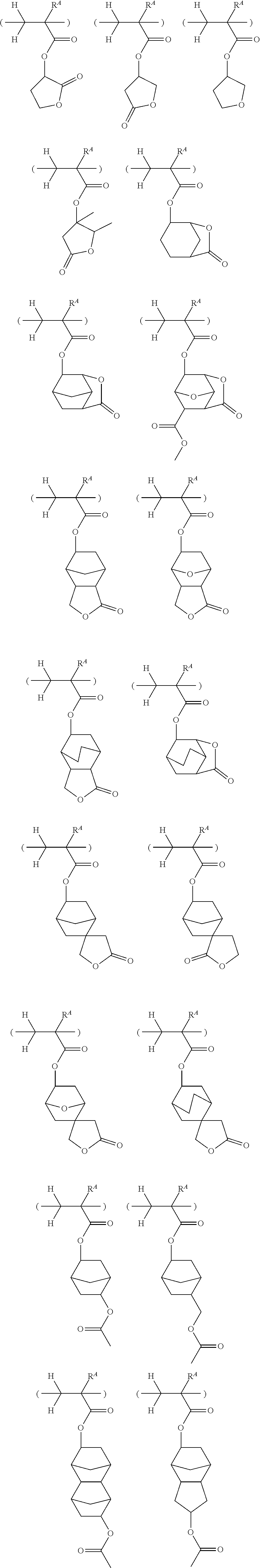

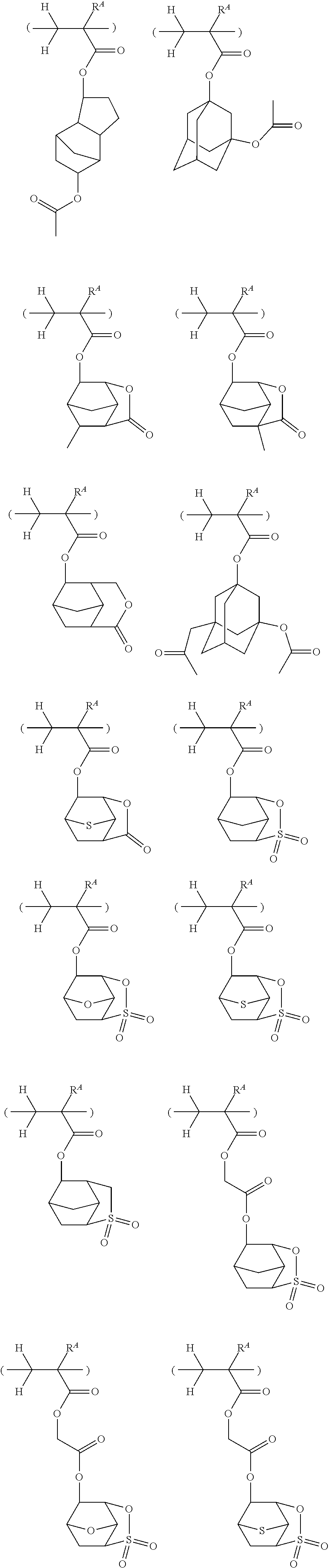

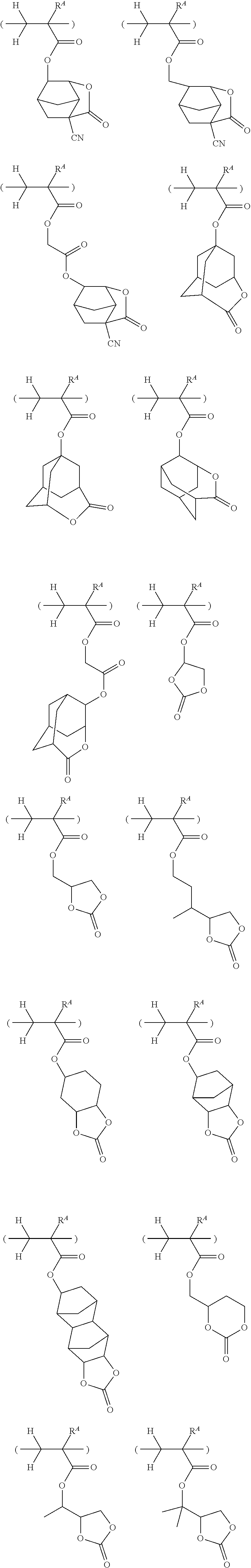

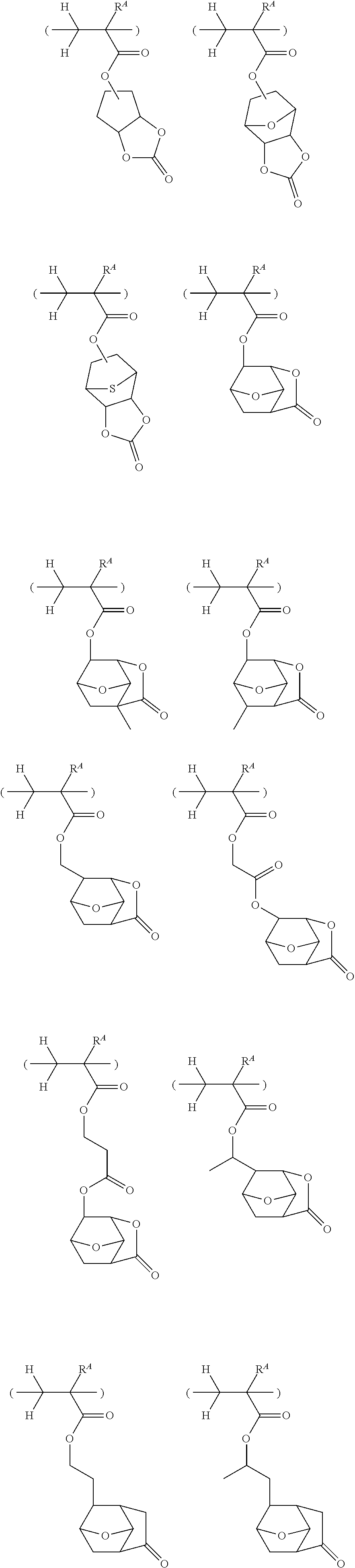

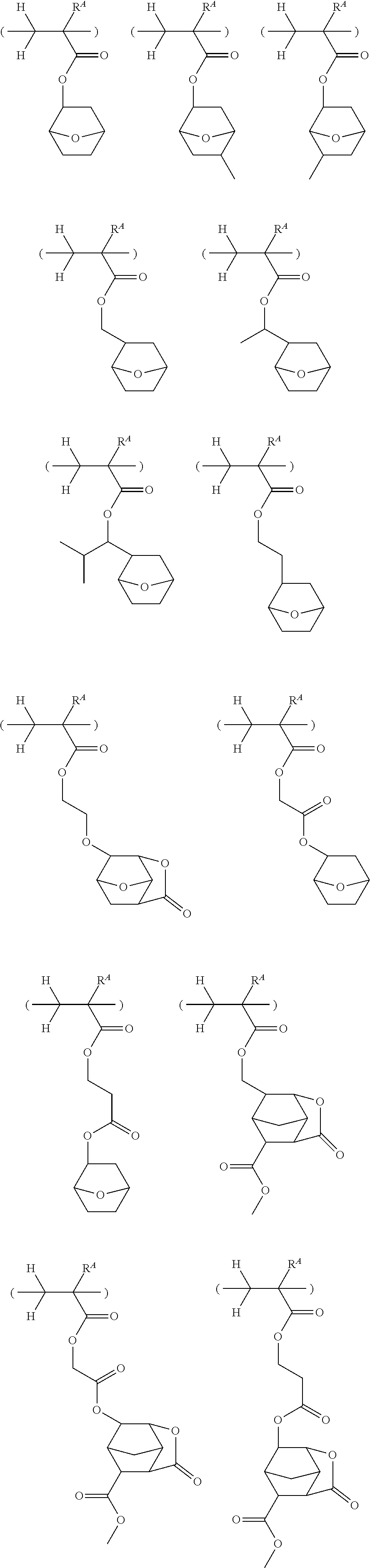

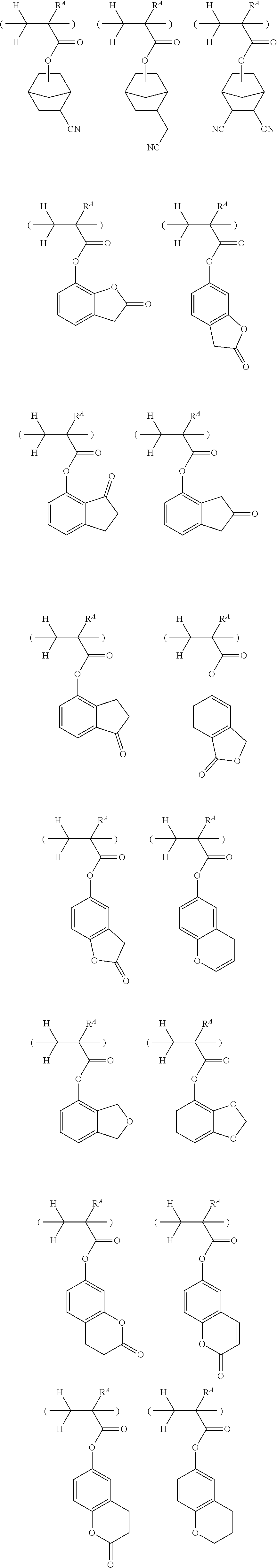

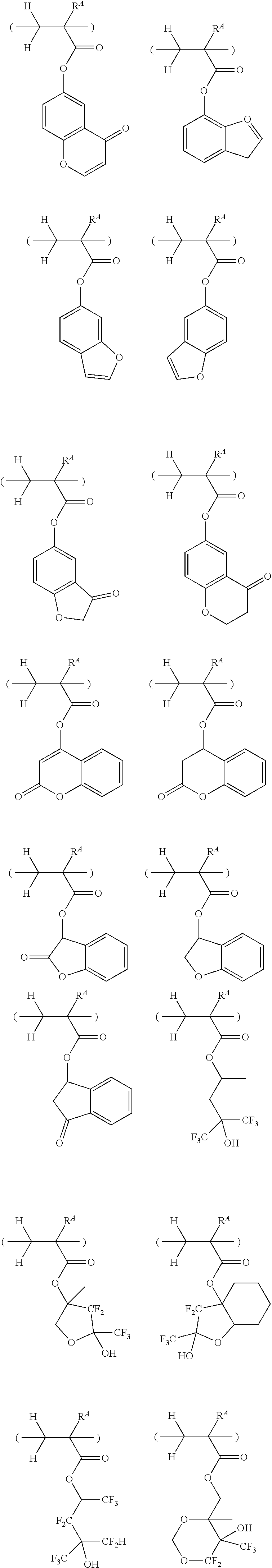

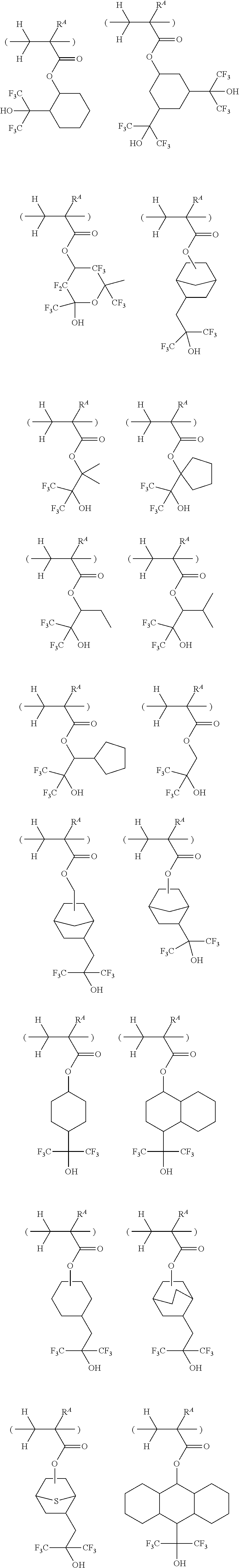

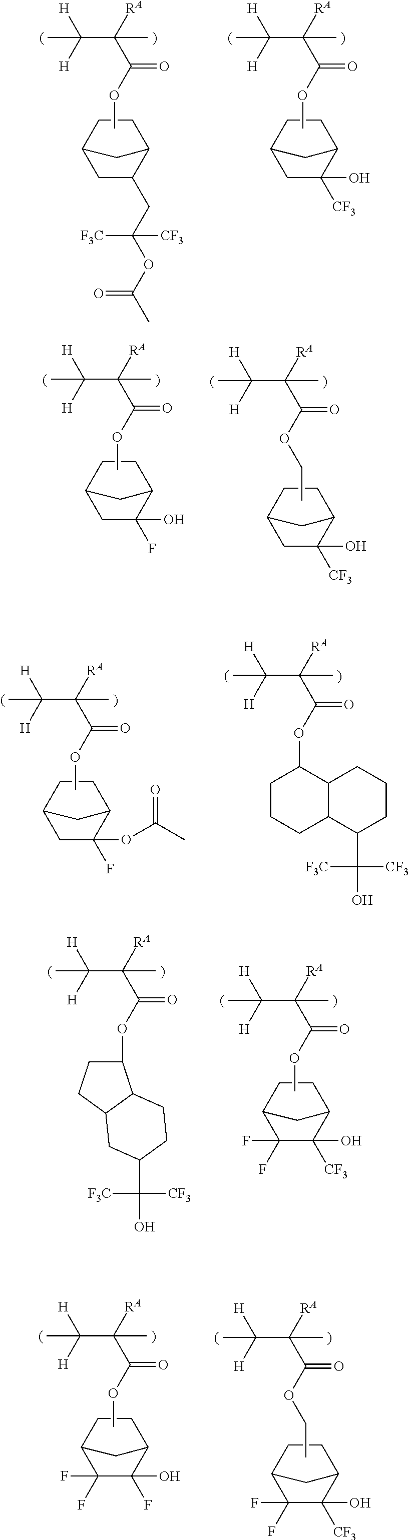

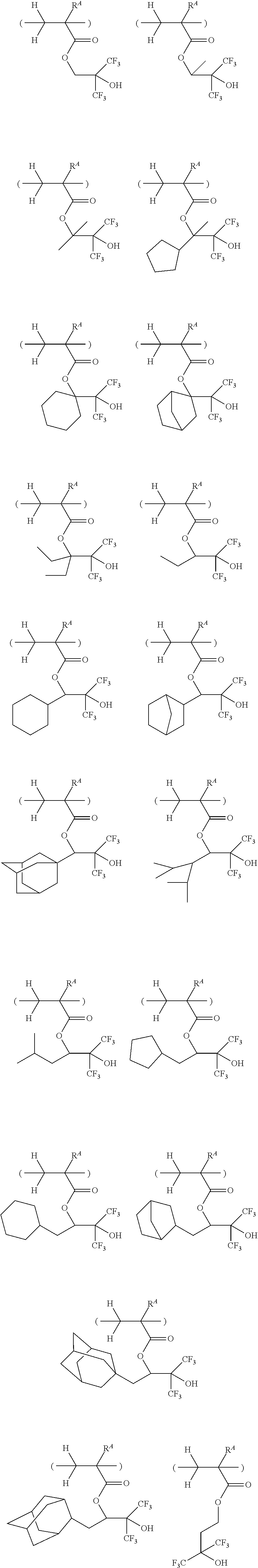

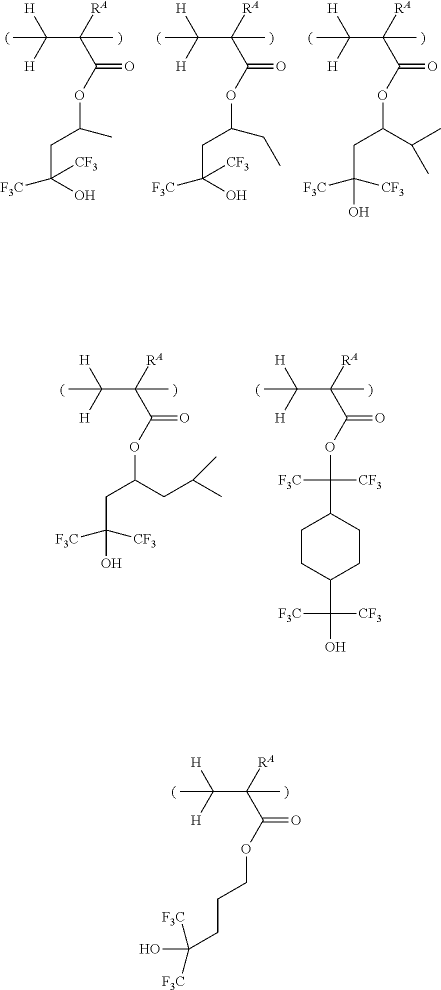

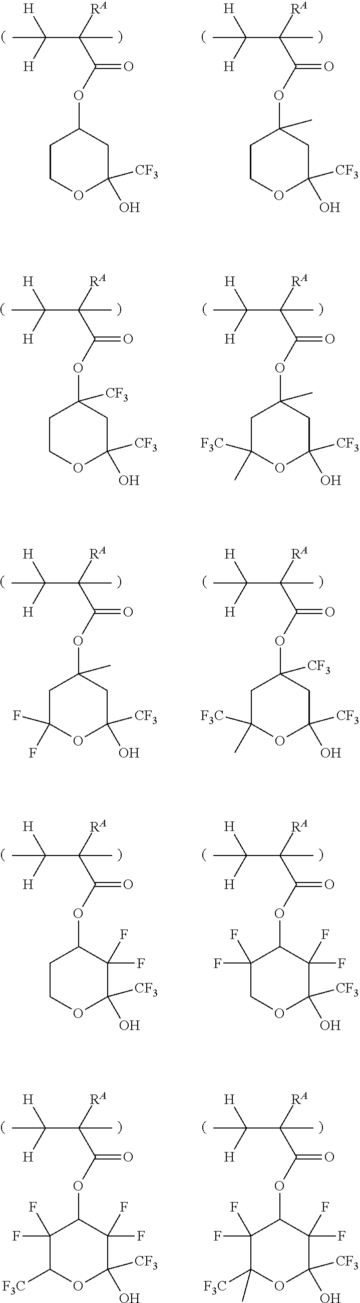

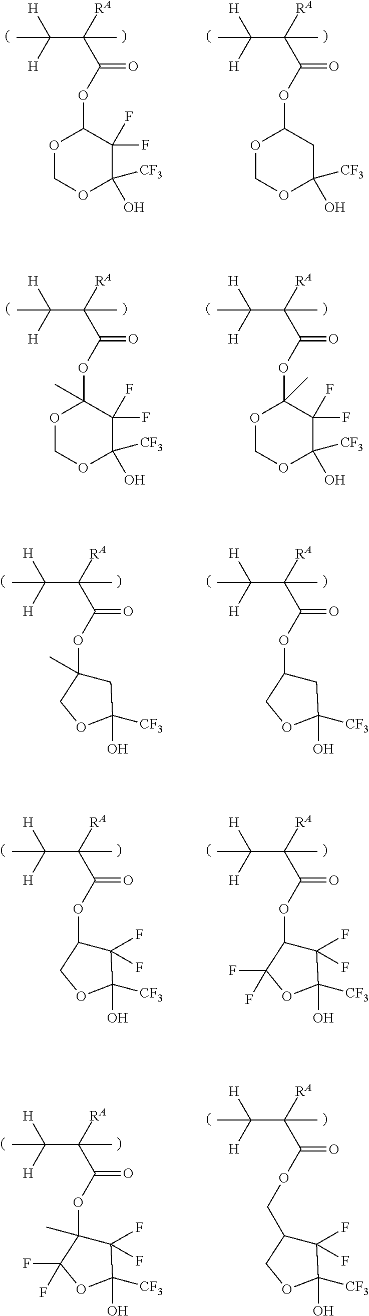

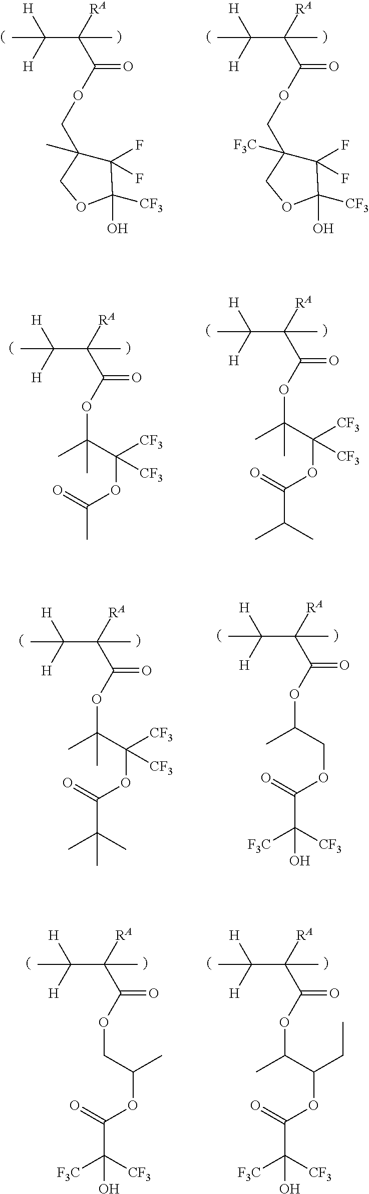

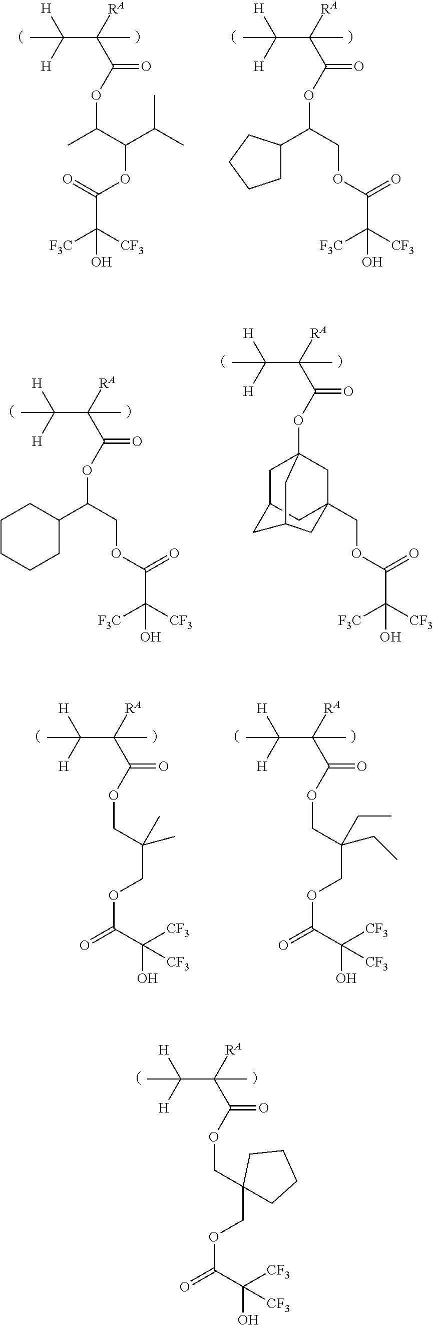

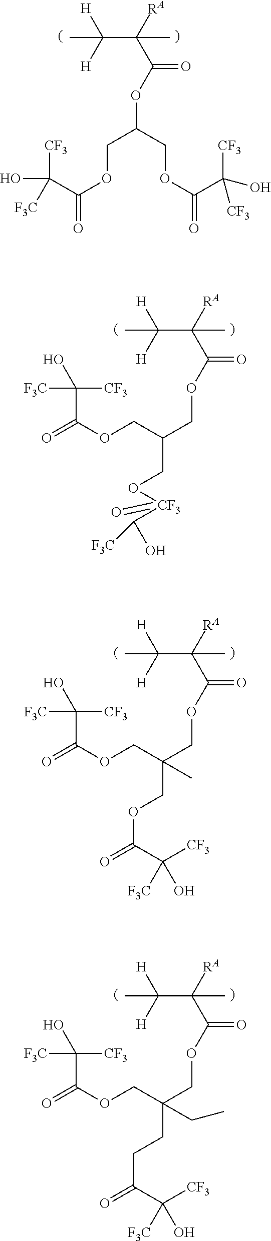

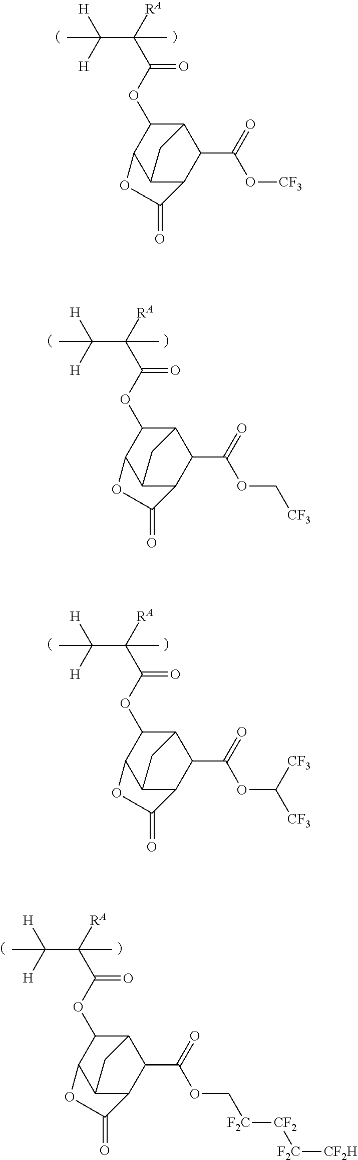

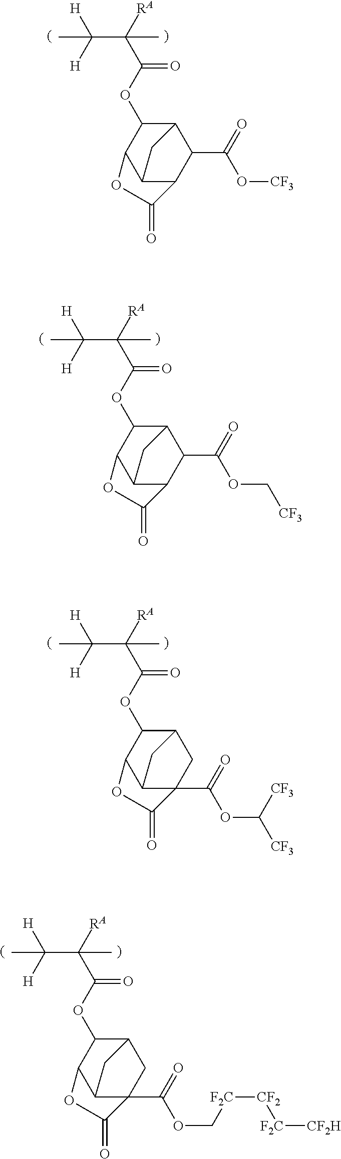

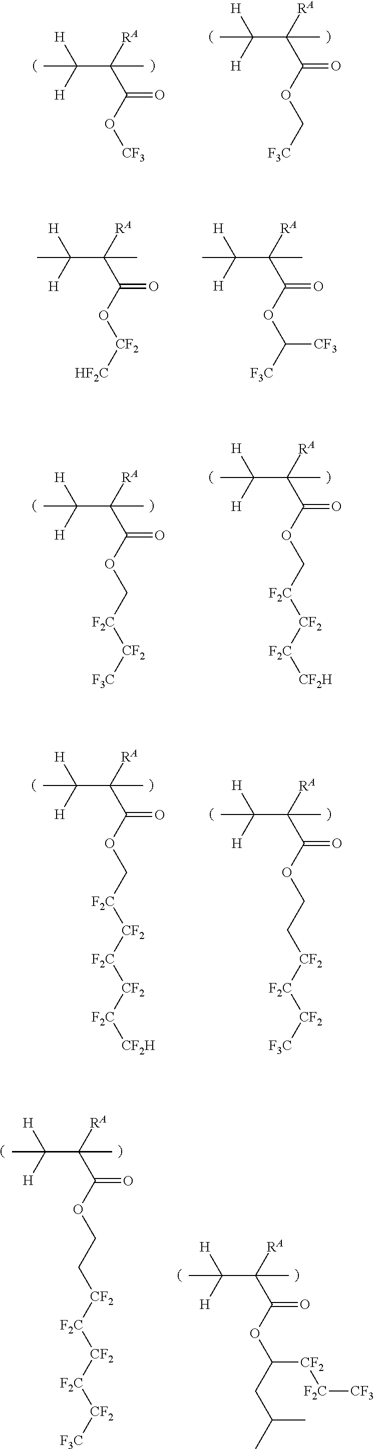

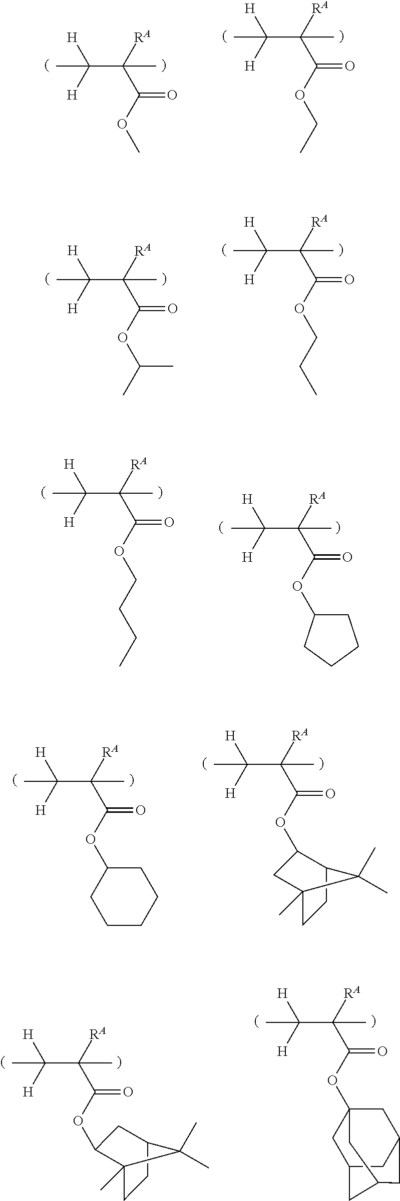

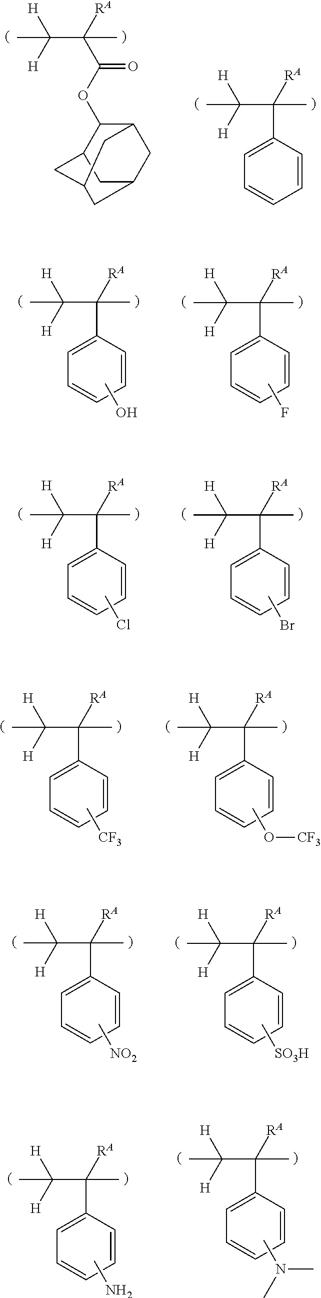

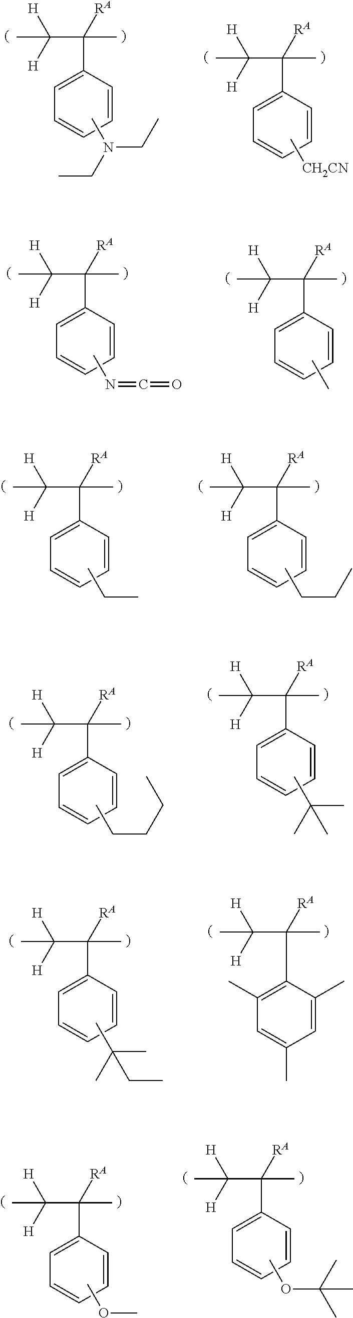

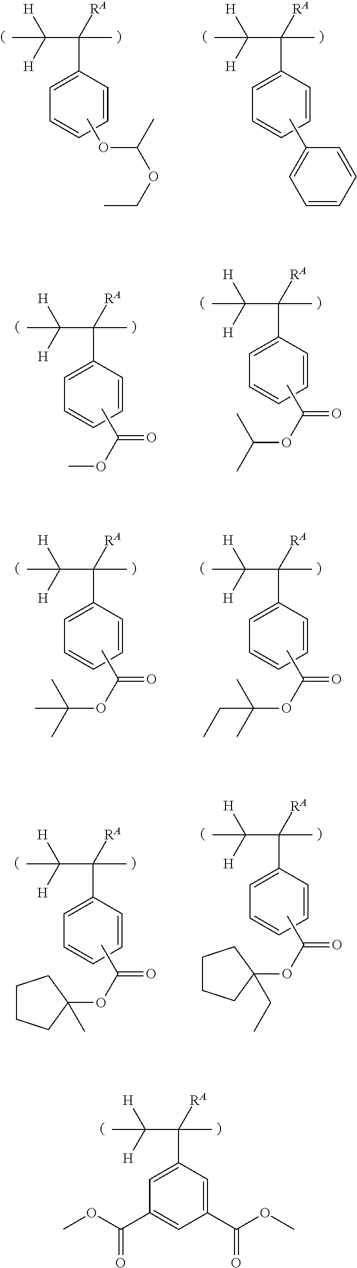

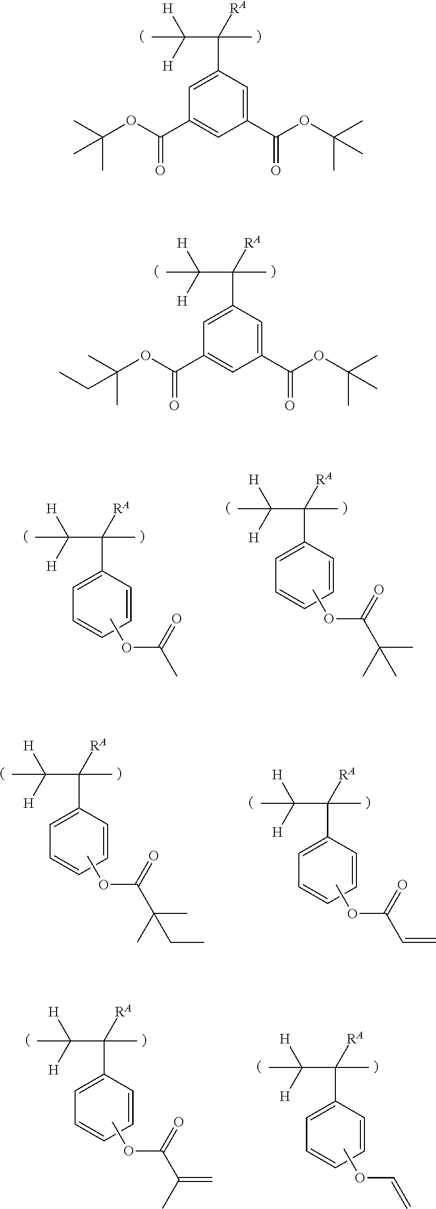

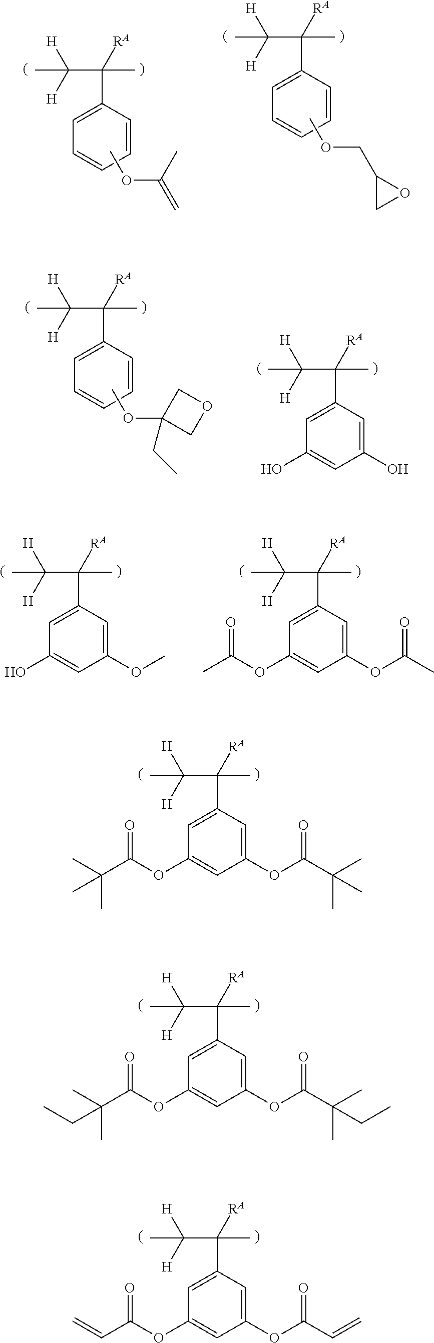

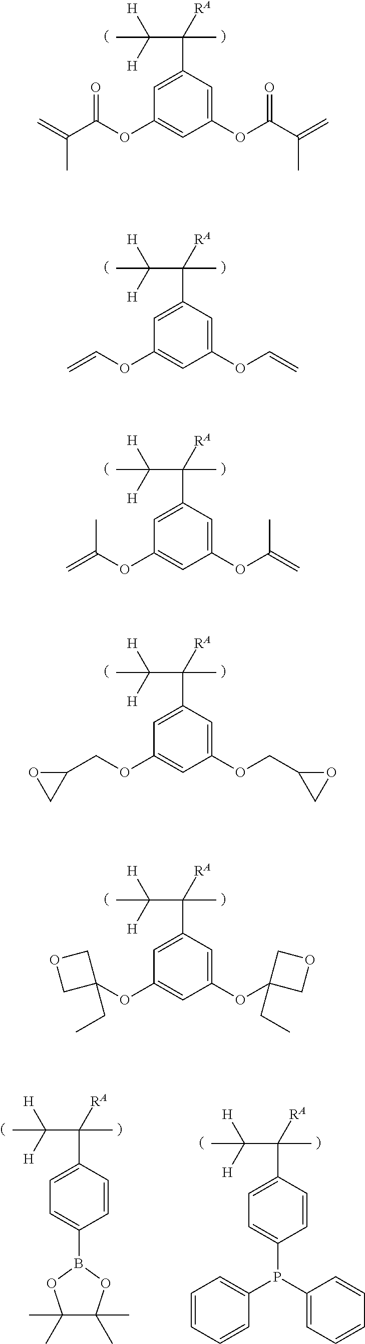

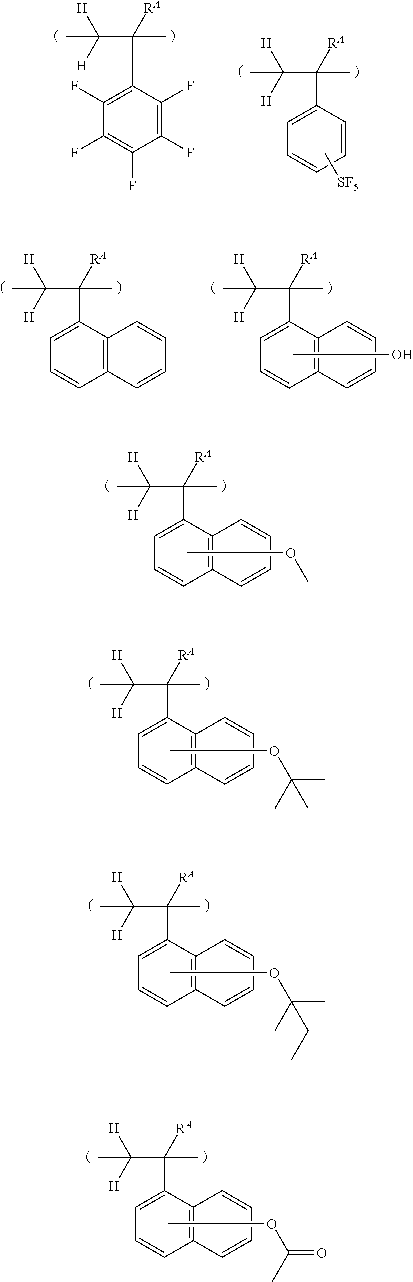

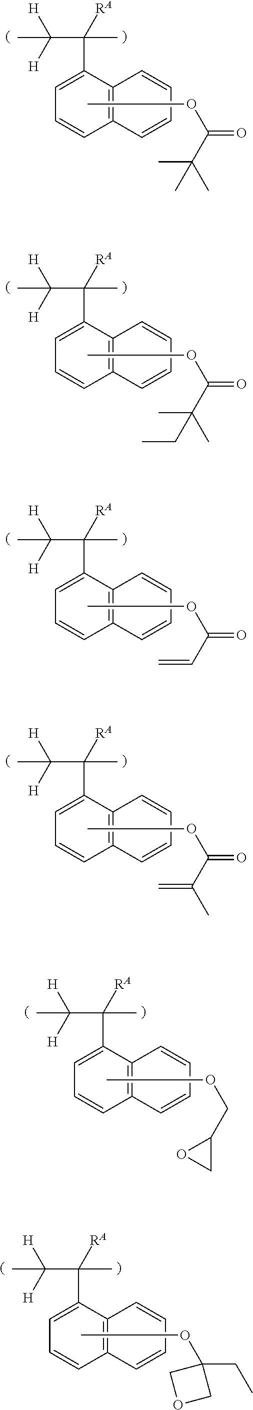

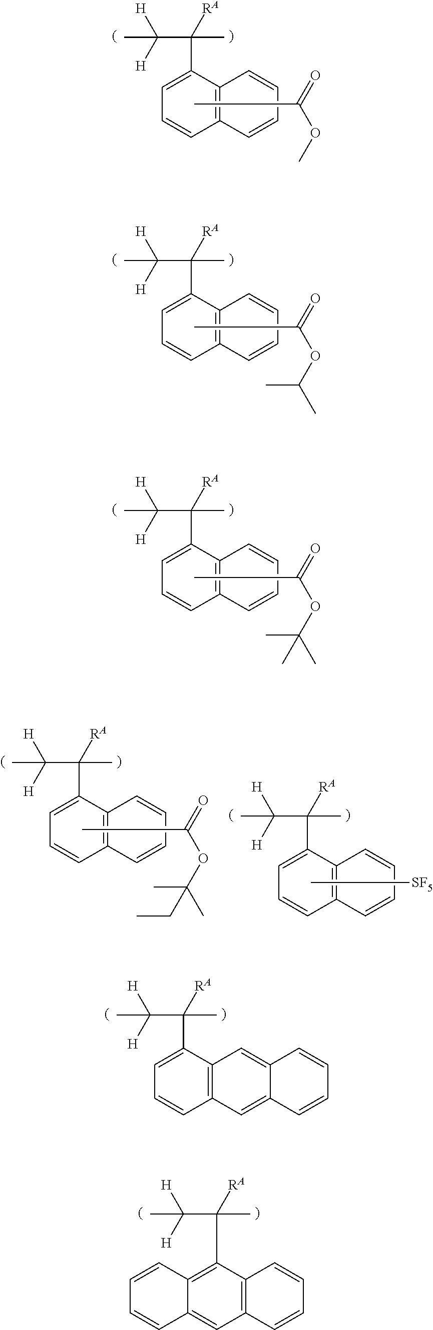

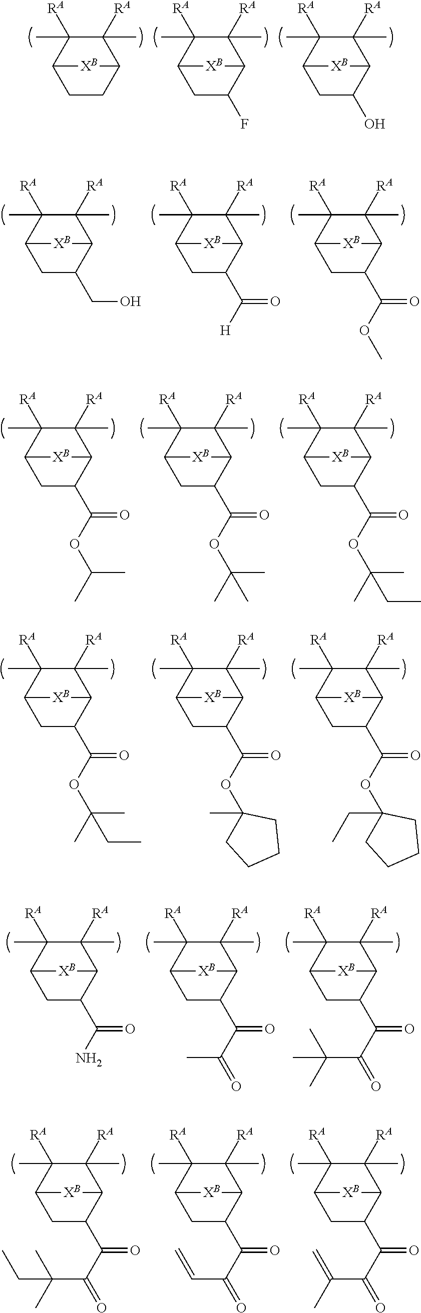

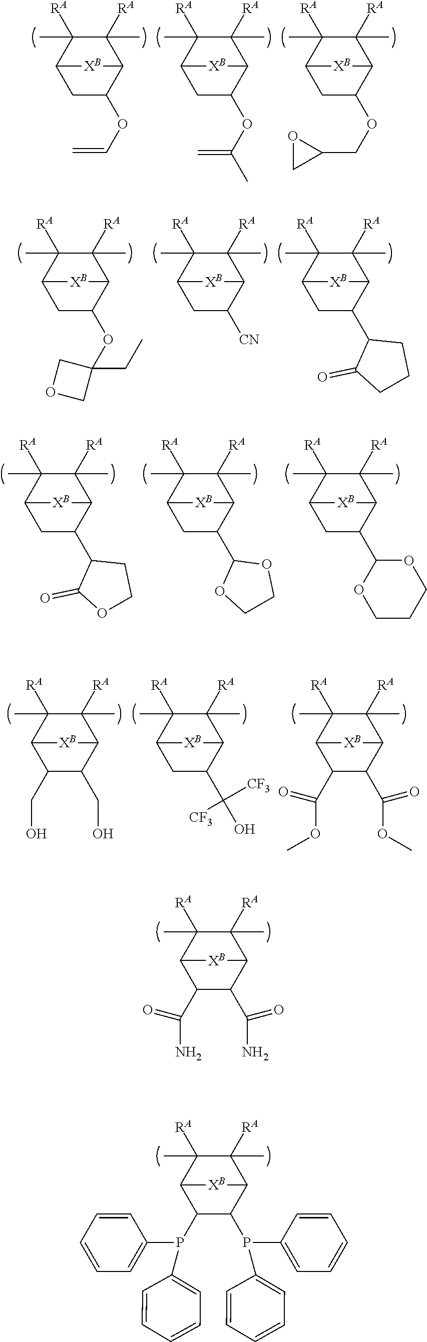

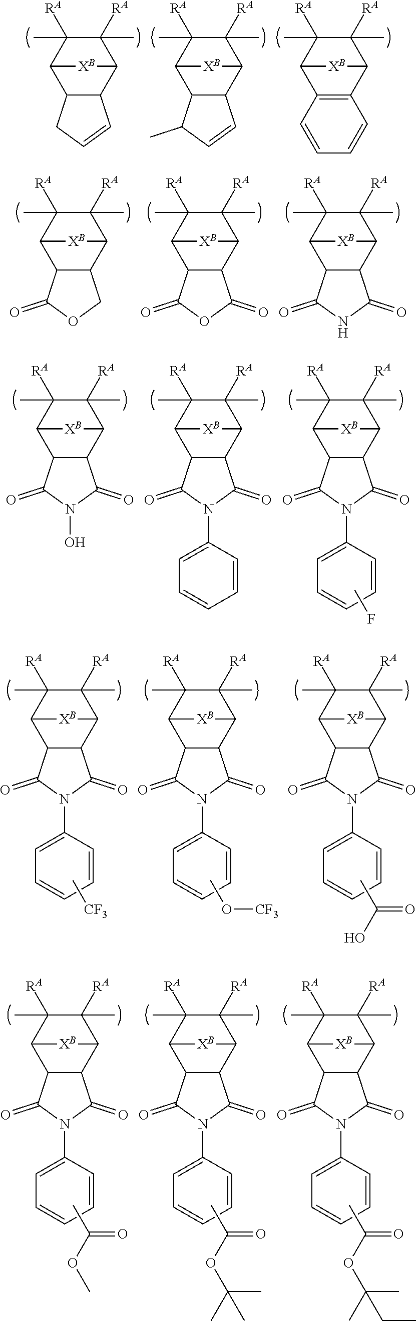

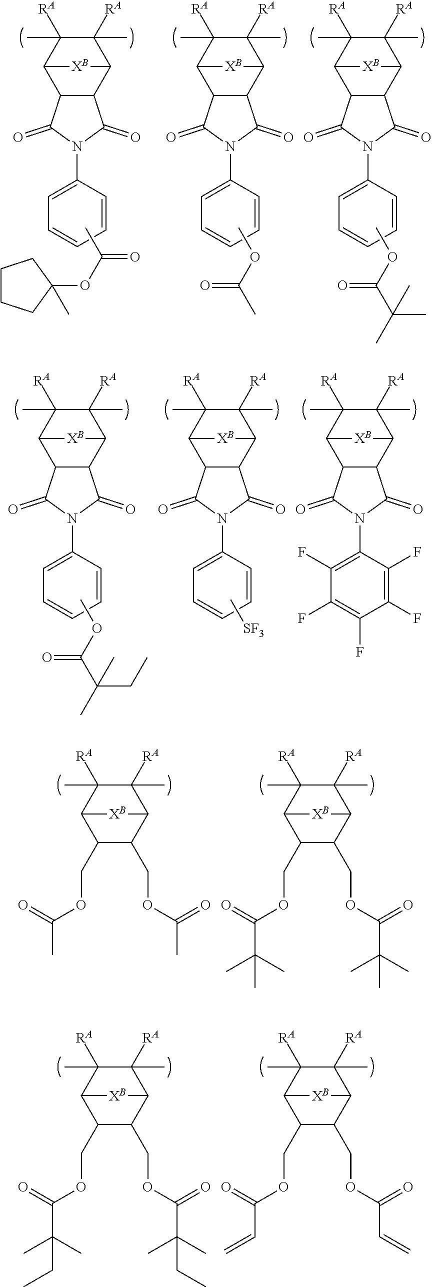

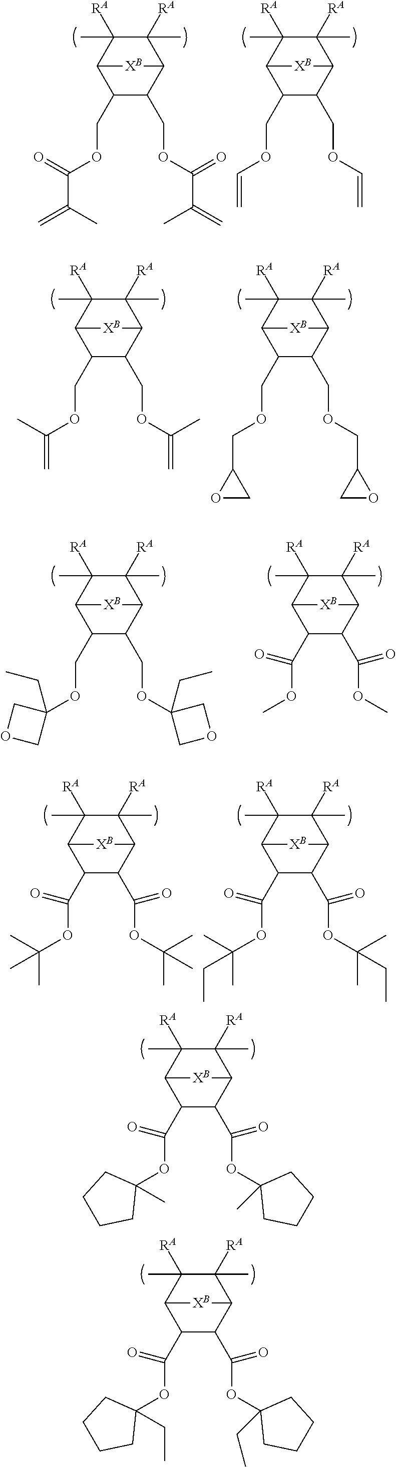

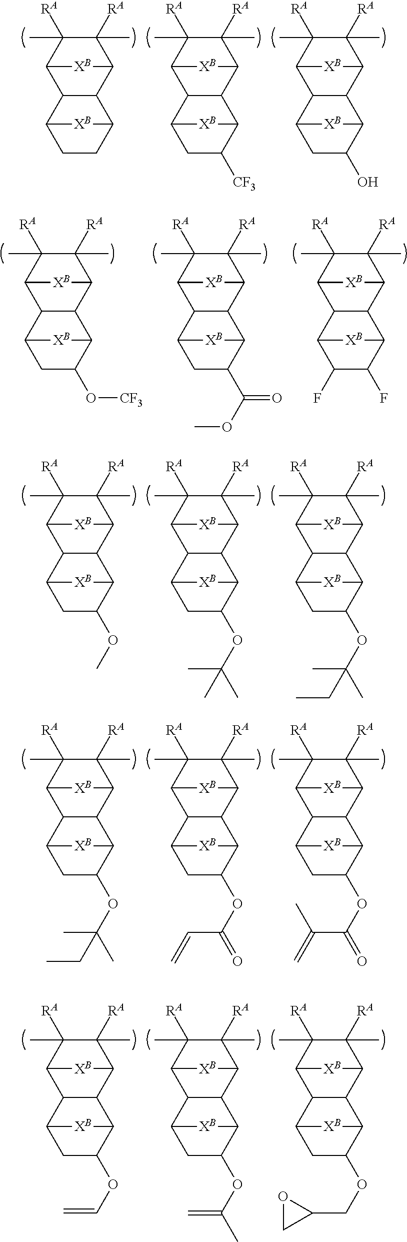

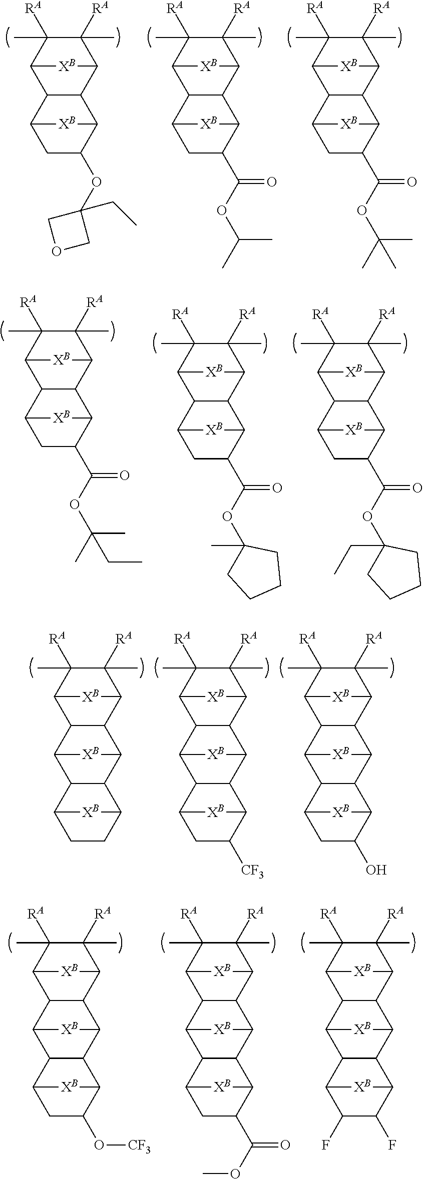

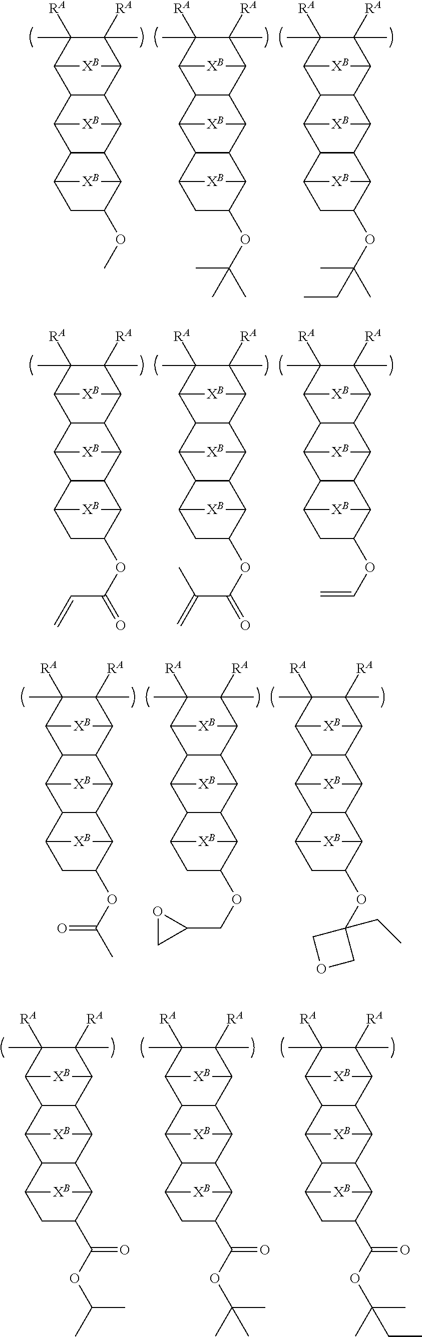

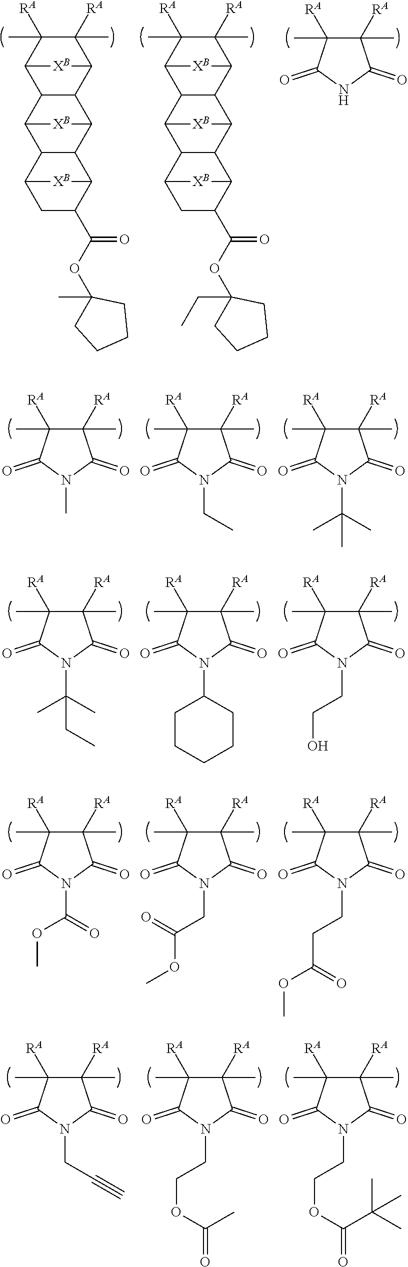

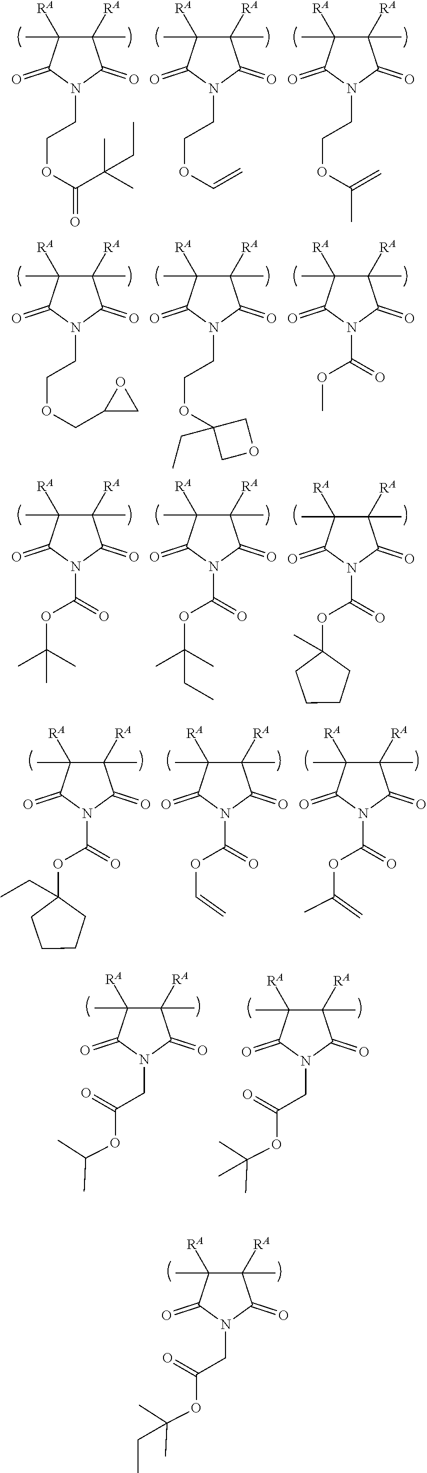

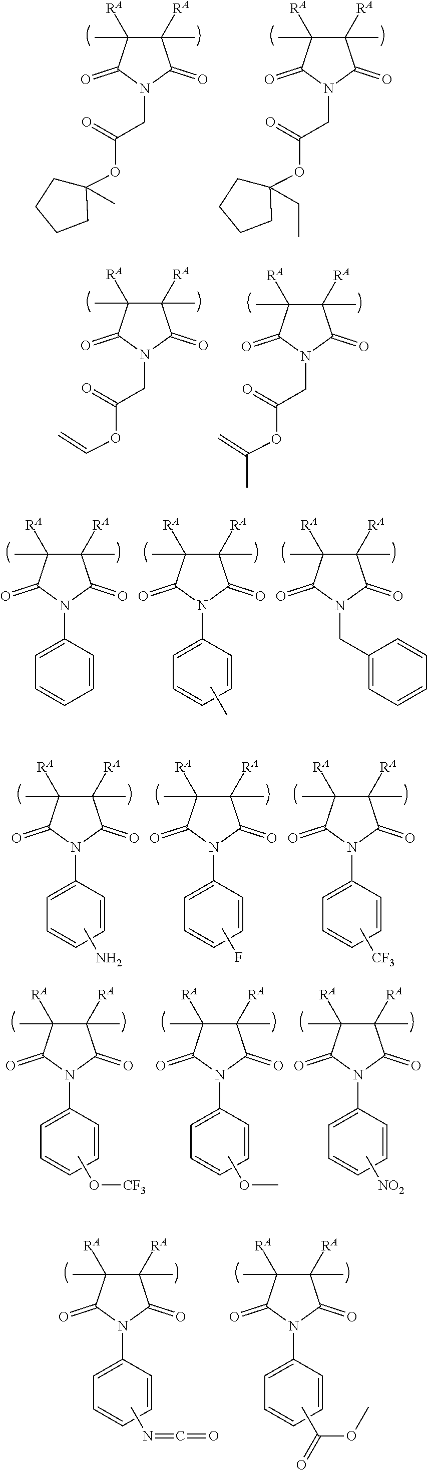

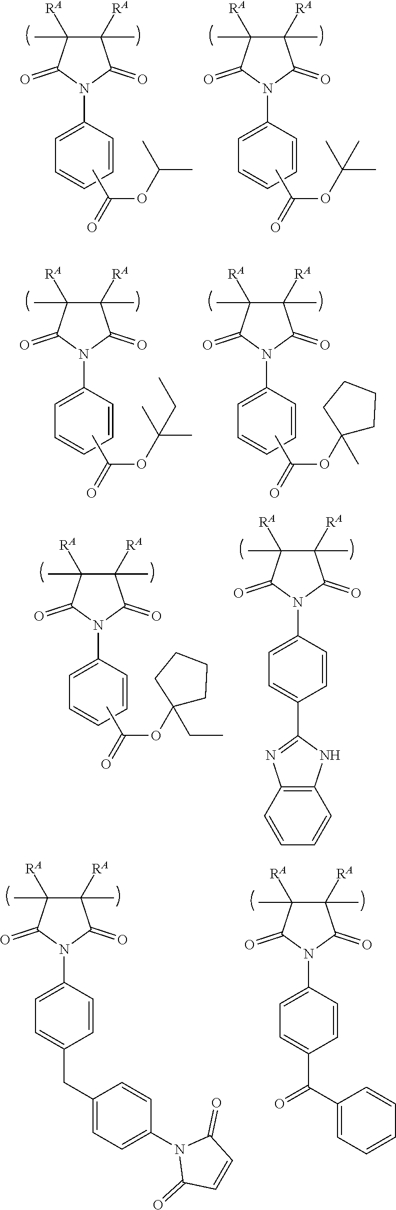

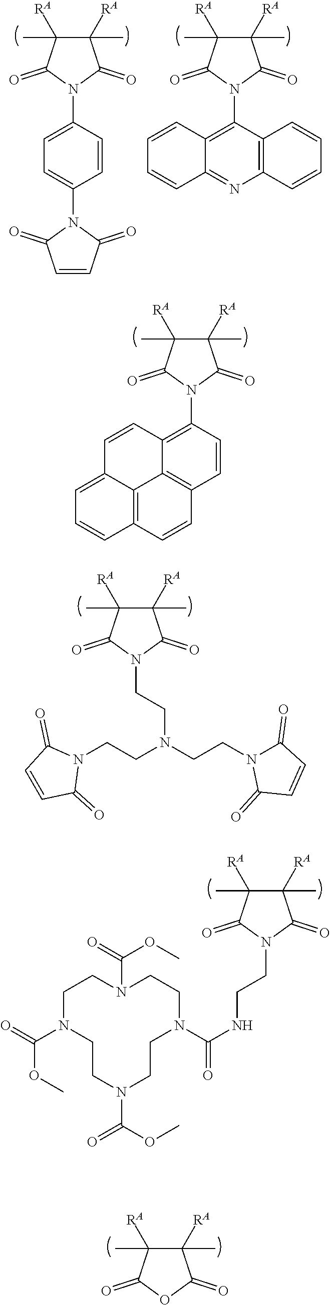

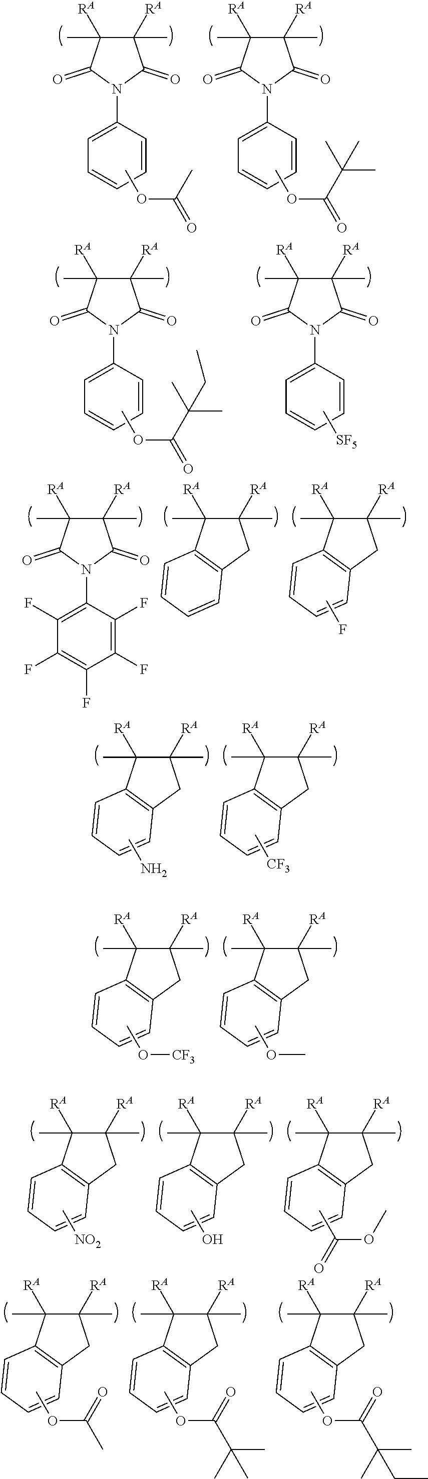

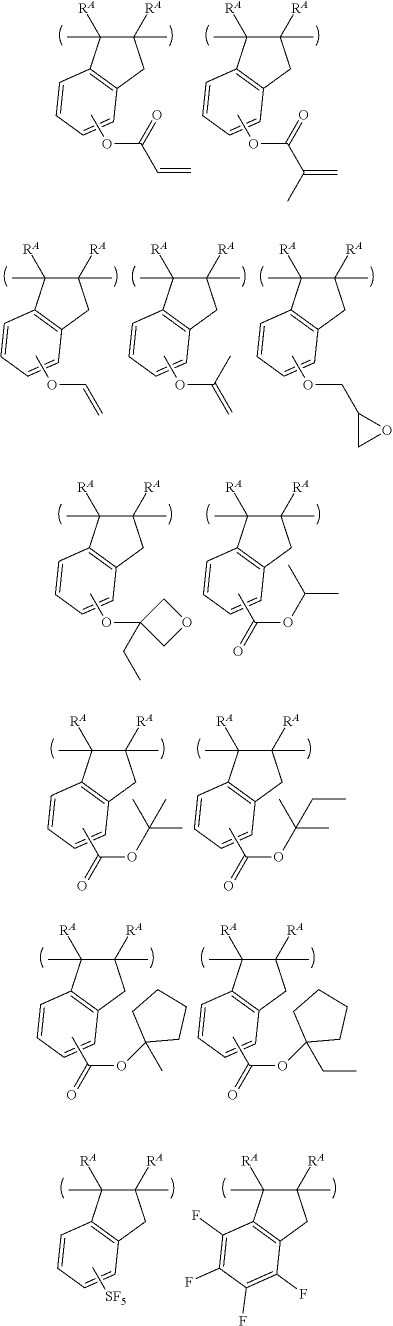

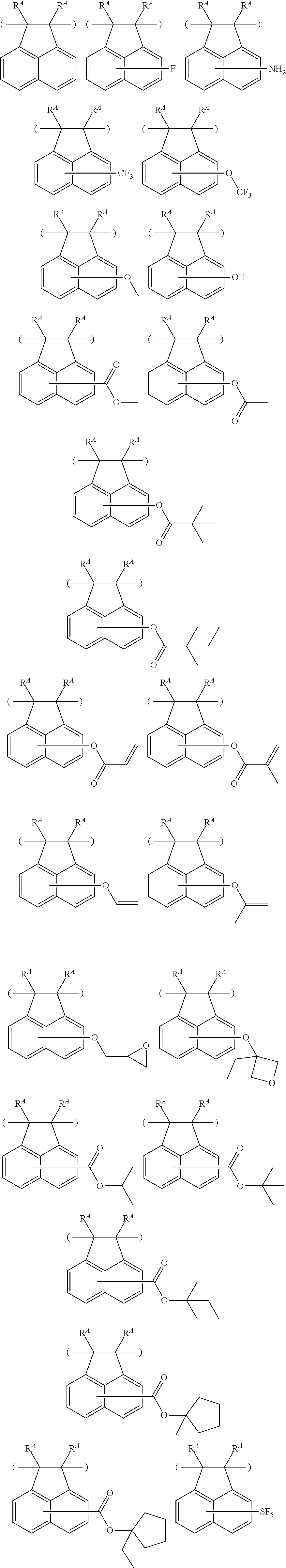

In the above-described resist composition, the content ratio of the hypervalent iodine compound to the carboxy-group-containing compound (the polymer including the repeating unit represented by the general formula (2) and/or the compound represented by the general formula (3)) (when the carboxy-group-containing compound is a carboxy-group-containing polymer, the content ratio of the hypervalent iodine compound (mol) to the carboxylic-acid-containing repeating unit (mol) in the polymer, and when the carboxy-group-containing compound is a monomolecular compound represented by the general formula (3), the content ratio of the hypervalent iodine compound (mol) to the monomolecular compound (mol)) is preferably “hypervalent iodine compound”: “carboxy-group-containing compound”=1:99 to 99:1, more preferably 10:90 to 90:10, and further preferably 20:80 to 80:20 in molar ratio. One kind of the hypervalent iodine compound may be used, or two or more kinds thereof may be used in combination. One kind of the carboxy-group-containing polymer may be used, or two or more kinds thereof having different composition ratios, weight-average molecular weights (Mw), and/or molecular weight distributions (Mw/Mn) may be used in combination. One kind of the monomolecular compound may be used, or two or more kinds thereof may be used in combination. One of the carboxy-group-containing polymer and the monomolecular compound may be used, or both may be used in combination.

In the carboxy-group-containing polymer, the content ratio (molar ratio) of the carboxy-group-containing repeating unit to the other repeating units is preferably “carboxy-group-containing repeating unit”:“other repeating units”=10:90 to 90:10, more preferably 15:85 to 85:15, and further preferably 20:80 to 80:20.

The carboxy-group-containing polymer preferably has a weight-average molecular weight (Mw) of 1000 to 500000, more preferably 3000 to 100000. Note that, in the present invention, weight-average molecular weight Mw and number-average molecular weight Mn are values measured in terms of standard polystyrene by gel permeation chromatography (GPC) using tetrahydrofuran (THF) as an eluent, and dispersity Mw/Mn is a value calculated therefrom.

Furthermore, if the carboxy-group-containing polymer has a wide molecular weight distribution (Mw/Mn), polymers having a lower molecular weight or a higher molecular weight are present, and therefore, there are risks that foreign substances may be found on the pattern after exposure, and the pattern shape may be degraded. Accordingly, as pattern rule is miniaturized, the influence of Mw and Mw/Mn is likely to be greater, and therefore, to obtain a resist composition that can be used suitably for a fine pattern size, the carboxy-group-containing polymer preferably has a narrow dispersity Mw/Mn of 1.00 to 2.00. Mw/Mn is preferably greater than 1.30, and the lower limit may be 1.40, 1.50, or 1.60, and the upper limit may be 1.70, 1.80, or 1.90.

Examples of methods for synthesizing the carboxy-group-containing polymer include a method of polymerizing a monomer to give a repeating unit described above by heating in an organic solvent in the presence of a radical polymerization initiator.

Specific examples of the organic solvent to be used in the polymerization reaction include toluene, benzene, THE, diethyl ether, dioxane, cyclohexane, cyclopentane, cyclopentanone, cyclohexanone, methyl ethyl ketone (MEK), propylene glycol monomethyl ether (PGME), propylene glycol monomethyl ether acetate (PGMEA), and γ-butyrolactone (GBL). Specific examples of the polymerization initiator include 2,2′-azobisisobutyronitrile (AIBN), 2,2′-azobis(2,4-dimethylvaleronitrile), dimethyl-2,2-azobis(2-methylpropionate), 1,1′-azobis(1-acetoxy-1-phenylethane), benzoyl peroxide, and lauroyl peroxide. The amount of the polymerization initiator to be added is preferably 0.01 to 25 mol % of the total amount of the monomers to be polymerized. The reaction temperature is preferably 50 to 150° C., more preferably 60 to 100° C. The reaction time is preferably 2 to 24 hours, and from the viewpoint of production efficiency, more preferably 2 to 12 hours.

The polymerization initiator may be added to the solution of the monomer and supplied to the reaction vessel, or a solution of the initiator may be prepared separately from the solution of the monomer, and each may be supplied to the reaction vessel independently. There is a possibility that the polymerization reaction may progress due to radicals generated from the initiator during waiting time and an ultra-high molecular weight polymer may be generated, and therefore, from the viewpoint of quality control, it is preferable to prepare each of the monomer solution and the initiator solution independently and add the solutions dropwise. Furthermore, to adjust the molecular weight, a known chain transfer agent, such as dodecyl mercaptan and 2-mercaptoethanol, may also be used. In this case, the amount of the chain transfer agent to be added is preferably 0.01 to 20 mol % of the total amount of the monomers to be polymerized.

Note that the amount of each monomer in the monomer solution can be, for example, set appropriately to achieve the preferable content ratios of the above-described repeating units.

### [Other Hypervalent Iodine Compounds]

The inventive resist composition may contain a hypervalent iodine compound represented by the following general formula (4) or (5) (hereinafter, also referred to as “other hypervalent iodine compounds”). When the inventive resist composition contains other hypervalent iodine compounds, reactivity to light can be controlled, and sensitivity can be adjusted.

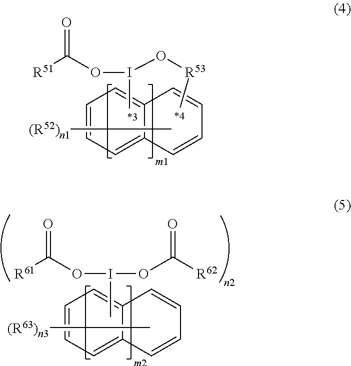

In the formulae, “m1” and “m2” each represent an integer of 0 to 2, “n1” representing an integer of 0 to 4 when “m1” is 0, an integer of 0 to 6 when “m1” is 1, and an integer of 0 to 8 when “m1” is 2, when “m2” is 0, “n2” representing an integer of 1 to 3, “n3” representing an integer of 0 to 5, and 1≤(n2+n3)≤6 being satisfied, when “m2” is 1, “n2” representing an integer of 1 to 3, “n3” representing an integer of 0 to 7, and 1≤(n2+n3)≤8 being satisfied, and when “m2” is 2, “n2” representing an integer of 1 to 3, “n3” representing an integer of 0 to 9, and 1≤(n2+n3)≤10 being satisfied; R⁵¹ represents a halogen atom or a hydrocarbyl group having 1 to 10 carbon atoms and optionally containing a heteroatom; R⁵² represents a halogen atom or a hydrocarbyl group having 1 to 40 carbon atoms and optionally containing a heteroatom, when “n1” is 2 to 8, the R⁵²s being identical to or different from each other and the R⁵²s optionally being bonded to each other to form a ring together with the carbon atoms of the aromatic ring bonded to the R⁵²s; R⁵³ represents a hydrocarbylene group having 1 to 10 carbon atoms and optionally containing a heteroatom; “*3” and “*4” each represent an attachment point to one of the carbon atoms of the aromatic ring in the formula, provided that “*3” and “*4” are bonded to adjacent carbon atoms of the aromatic ring; R⁶¹ and R⁶² each independently represent a halogen atom or a hydrocarbyl group having 1 to 10 carbon atoms and optionally containing a heteroatom, the R⁶¹ and the R⁶² optionally being bonded to each other to form a ring together with the carbon atoms bonded thereto and the atoms between the carbon atoms, and when “n2” is 2 or 3, the Rols and the R⁶²s being identical to or different from each other; and R⁶³ represents a halogen atom or a hydrocarbyl group having 1 to 40 carbon atoms and optionally containing a heteroatom, when “n3” is 2 to 9, the R⁶³s being identical to or different from each other and the R⁶³s optionally being bonded to each other to form a ring together with the carbon atoms of the aromatic ring bonded to the R⁶³s.

In the general formula (4) and the general formula (5), “m1” and “m2” each represent an integer of 0 to 2. “n1” represents an integer of 0 to 4 when “m1” is 0, an integer of 0 to 6 when “m1” is 1, and an integer of 0 to 8 when “m1” is 2.

When “m2” is 0, “n2” represents an integer of 1 to 3, “n3” represents an integer of 0 to 5, and 1≤(n2+n3)≤6 is satisfied.

When “m2” is 1, “n2” represents an integer of 1 to 3, “n3” represents an integer of 0 to 7, and 1≤(n2+n3)≤8 is satisfied.

When “m2” is 2, “n2” represents an integer of 1 to 3, “n3” represents an integer of 0 to 9, and 1≤(n2+n3)≤10 is satisfied.

R⁵¹ represents a halogen atom or a hydrocarbyl group having 1 to 10 carbon atoms and optionally containing a heteroatom.

R⁵² represents a halogen atom or a hydrocarbyl group having 1 to 40 carbon atoms and optionally containing a heteroatom, when “n1” is 2 to 8, the R⁵²s being identical to or different from each other and the R⁵²s optionally being bonded to each other to form a ring together with the carbon atoms of the aromatic ring bonded to the R⁵²s.

R⁵³ represents a hydrocarbylene group having 1 to 10 carbon atoms and optionally containing a heteroatom.

“*3” and “*4” each represent an attachment point to one of the carbon atoms of the aromatic ring in the formula, provided that “*3” and “*4” are bonded to adjacent carbon atoms of the aromatic ring.

R⁶¹ and R⁶² each independently represent a halogen atom or a hydrocarbyl group having 1 to 10 carbon atoms and optionally containing a heteroatom, the R⁶¹ and the R⁶² optionally being bonded to each other to form a ring together with the carbon atoms bonded thereto and the atoms between the carbon atoms.

R⁶³ represents a halogen atom or a hydrocarbyl group having 1 to 40 carbon atoms and optionally containing a heteroatom, when “n3” is 2 to 9, the R⁶³s being identical to or different from each other and the R⁶³s optionally being bonded to each other to form a ring together with the carbon atoms of the aromatic ring bonded to the R⁶³s.

In the general formula (4), “m1” represents an integer of 0 to 2. “n1” represents an integer of 0 to 4 when “m1” is 0, an integer of 0 to 6 when “m1” is 1, and an integer of 0 to 8 when “m1” is 2. “n1” is preferably 0, 1, 2, 3, or 4, more preferably 0, 1, 2, or 3, further preferably 0, 1, or 2, and most preferably 0 or 1.

In the general formula (4), R⁵¹ represents a halogen atom or a hydrocarbyl group having 1 to 10 carbon atoms and optionally containing a heteroatom. Specific examples of the halogen atom include a fluorine atom, a chlorine atom, a bromine atom, and an iodine atom. The hydrocarbyl group having 1 to 10 carbon atoms may be saturated or unsaturated, and may be linear, branched, or cyclic. Specific examples thereof include: alkyl groups having 1 to 10 carbon atoms, such as a methyl group, an ethyl group, an n-propyl group, an isopropyl group, an n-butyl group, an isobutyl group, a sec-butyl group, a tert-butyl group, an n-pentyl group, a tert-pentyl group, an n-hexyl group, an n-octyl group, a 2-ethylhexyl group, an n-nonyl group, and an n-decyl group; cyclic saturated hydrocarbyl groups having 3 to 10 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.0^{2,6}]decanyl group, and an adamantyl group; alkenyl groups having 2 to 10 carbon atoms, such as a vinyl group and an allyl group; aryl groups having 6 to 10 carbon atoms, such as a phenyl group and a naphthyl group; and groups which are combinations of these groups. Furthermore, part or all of the hydrogen atoms of the hydrocarbyl group may be substituted with a group containing a heteroatom, such as an oxygen atom, a sulfur atom, a nitrogen atom, or a halogen atom, and part of the —CH₂— of the hydrocarbyl group may be substituted with a group containing a heteroatom such as an oxygen atom, a sulfur atom, or a nitrogen atom. The resulting hydrocarbyl group may contain a hydroxy group, a cyano group, a halogen atom, a carbonyl group, an ether bond, a thioether bond, an ester bond, a sulfonic acid ester bond, a carbonate bond, a carbamate bond, a lactone ring, a sultone ring, a carboxylic anhydride (—C(═O)—O—C(═O)—), etc. As the R⁵¹, a hydrocarbyl group having 1 to 4 carbon atoms or a fluorinated hydrocarbyl group having 1 to 4 carbon atoms is preferable, and a hydrocarbyl group having 1 to 4 carbon atoms is more preferable.

In the general formula (4), R⁵² represents a halogen atom or a hydrocarbyl group having 1 to 40 carbon atoms and optionally containing a heteroatom. Specific examples of the halogen atom include a fluorine atom, a chlorine atom, a bromine atom, and an iodine atom. The hydrocarbyl group having 1 to 40 carbon atoms may be saturated or unsaturated, and may be linear, branched, or cyclic. Specific examples thereof include: alkyl groups having 1 to 40 carbon atoms, such as a methyl group, an ethyl group, an n-propyl group, an isopropyl group, an n-butyl group, an isobutyl group, a sec-butyl group, a tert-butyl group, an n-pentyl group, a tert-pentyl group, an n-hexyl group, an n-octyl group, a 2-ethylhexyl group, an n-nonyl group, and an n-decyl group; cyclic saturated hydrocarbyl groups having 3 to 40 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.0^{2,6}]decanyl group, an adamantyl group, and an adamantylmethyl group; and aryl groups having 6 to 40 carbon atoms, such as a phenyl group, a naphthyl group, and an anthracenyl group. Furthermore, part or all of the hydrogen atoms of the hydrocarbyl group may be substituted with a group containing a heteroatom, such as an oxygen atom, a sulfur atom, a nitrogen atom, or a halogen atom, and part of the —CH₂— of the hydrocarbyl group may be substituted with a group containing a heteroatom such as an oxygen atom, a sulfur atom, or a nitrogen atom. The resulting hydrocarbyl group may contain a hydroxy group, a cyano group, a halogen atom, a carbonyl group, an ether bond, a thioether bond, an ester bond, a sulfonic acid ester bond, a carbonate bond, a carbamate bond, a lactone ring, a sultone ring, a carboxylic anhydride (—C(═O)—O—C(═O)—), etc. When “n1” is 2 to 8, the R⁵²s are identical to or different from each other and the R⁵²s are optionally bonded to each other to form a ring together with the carbon atoms of the aromatic ring bonded to the R⁵²s.

In the general formula (4), R⁵³ represents a hydrocarbylene group having 1 to 10 carbon atoms and optionally containing a heteroatom. The hydrocarbylene group having 1 to 10 carbon atoms may be saturated or unsaturated, and may be linear, branched, or cyclic. Specific examples thereof include: alkylene groups having 1 to 10 carbon atoms, such as a methanediyl group, an ethane-1,1-diyl group, an ethane-1,2-diyl group, a propane-1,1-diyl group, a propane-1,2-diyl group, a propane-1,3-diyl group, a propane-2,2-diyl group, a butane-2,3-diyl group, a butane-1,4-diyl group, a 2-methylpropane-1,2-diyl group, a pentane-1,5-diyl group, a hexane-1,6-diyl group, a heptane-1,7-diyl group, an octane-1,8-diyl group, a nonane-1,9-diyl group, and a decane-1,10-diyl group; cyclic saturated hydrocarbylene groups having 3 to 10 carbon atoms, such as a cyclopentanediyl group, a cyclohexanediyl group, a norbornanediyl group, an adamantanediyl group, and a tricyclo[5.2.1.0^{2,6}]decanediyl group; alkenylene groups having 2 to 10 carbon atoms, such as a vinylene group and a propynylene group; arylene groups having 6 to 10 carbon atoms, such as a phenylene group, a methylphenylene group, an ethylphenylene group, an n-propylphenylene group, an isopropylphenylene group, an n-butylphenylene group, and a naphthylene group; and groups which are combinations of these groups. Furthermore, part or all of the hydrogen atoms of the hydrocarbylene group may be substituted with a group containing a heteroatom, such as an oxygen atom, a sulfur atom, a nitrogen atom, or a halogen atom, and part of the —CH₂— of the hydrocarbylene group may be substituted with a group containing a heteroatom such as an oxygen atom, a sulfur atom, or a nitrogen atom. The resulting hydrocarbylene group may contain a hydroxy group, a cyano group, a halogenated alkyl group, a halogen atom, a carbonyl group, an ether bond, a thioether bond, an ester bond, a sulfonic acid ester bond, a carbonate bond, a carbamate bond, a lactone ring, a sultone ring, a carboxylic anhydride (—C(═O)—O—C(═O)—), etc. As R⁵³, a carbonyl group, a hydrocarbylene group having 1 to 4 carbon atoms, or a fluorinated hydrocarbylene group having 1 to 4 carbon atoms is preferable.

In the general formula (4), “*3” and “*4” each represent an attachment point to one of the carbon atoms of the aromatic ring in the general formula (4), provided that “*3” and “*4” are bonded to adjacent carbon atoms of the aromatic ring. As combinations of such “*3”, “*4”, and “m1”, the seven cases shown below are possible.

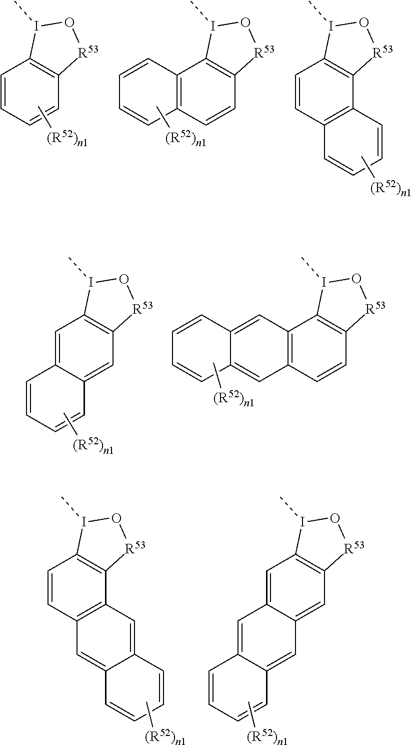

In the formulae, “n1”, R⁵², and R⁵³ are as defined above. A broken line represents an attachment point to R⁵¹—C(═O)—O—.

Note that the R⁵² and the R⁵³ can substitute any position in the aromatic rings in the above formulae.

Specific examples of the hypervalent iodine compound represented by the general formula (4) include the following, but are not limited thereto. Note that, in the following formulae, Me represents a methyl group.

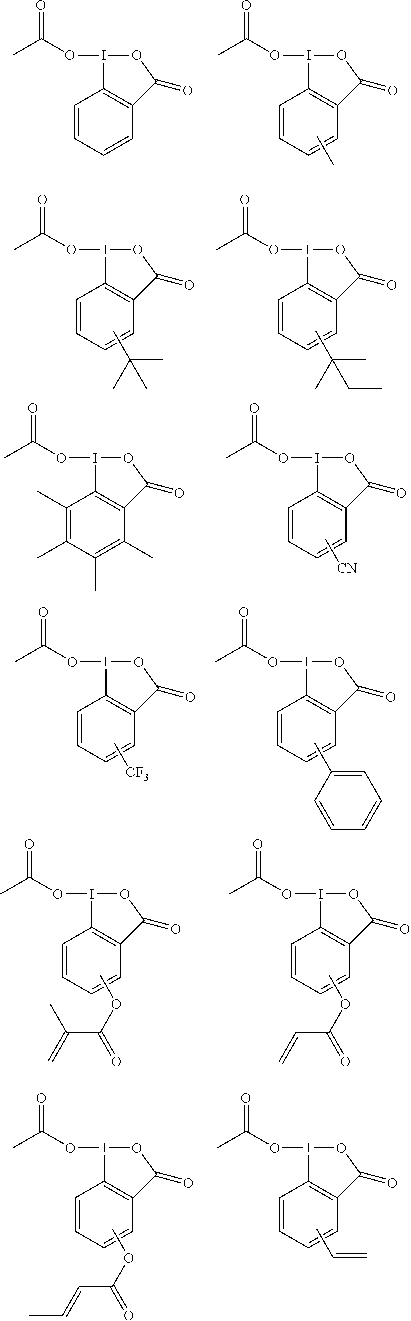

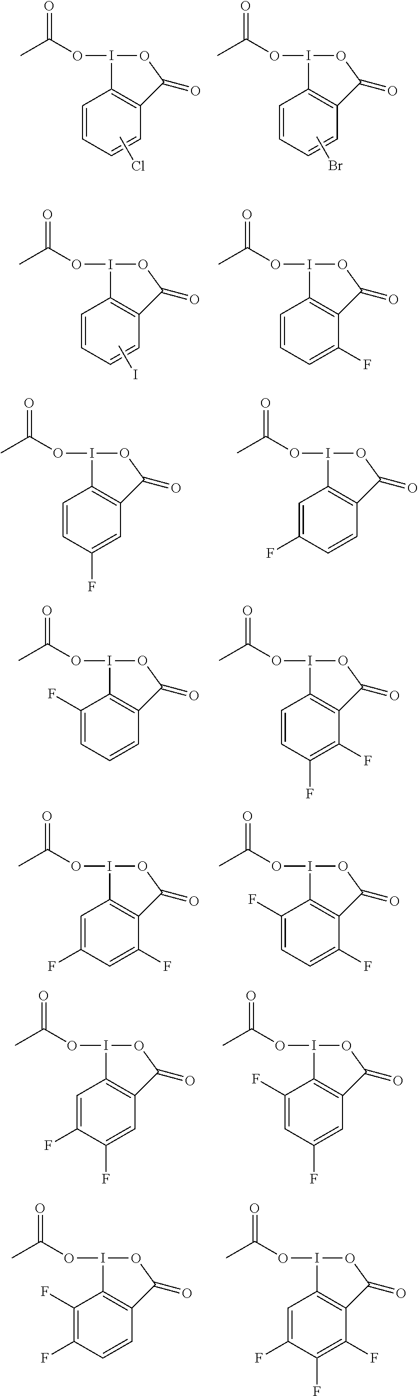

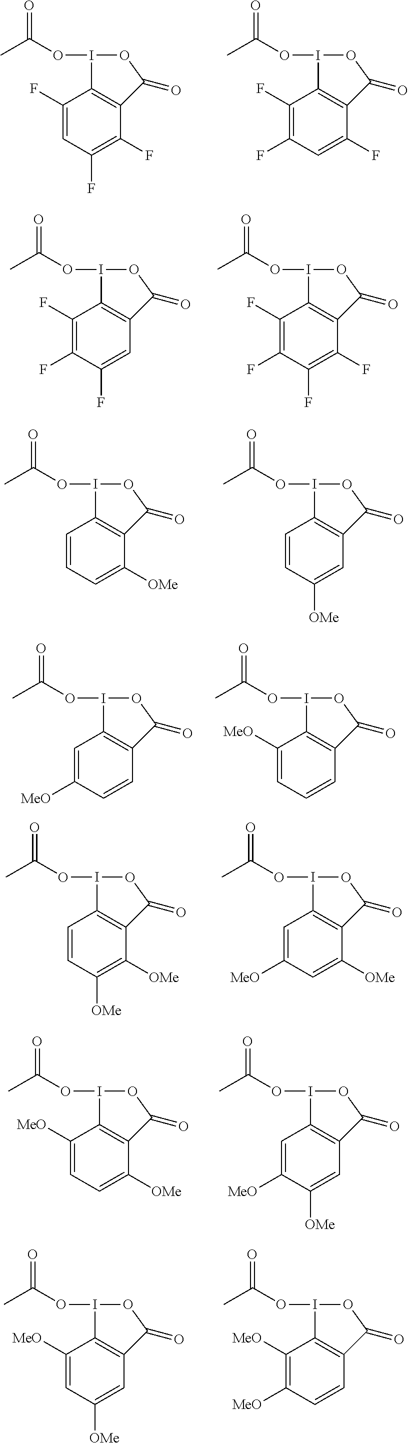

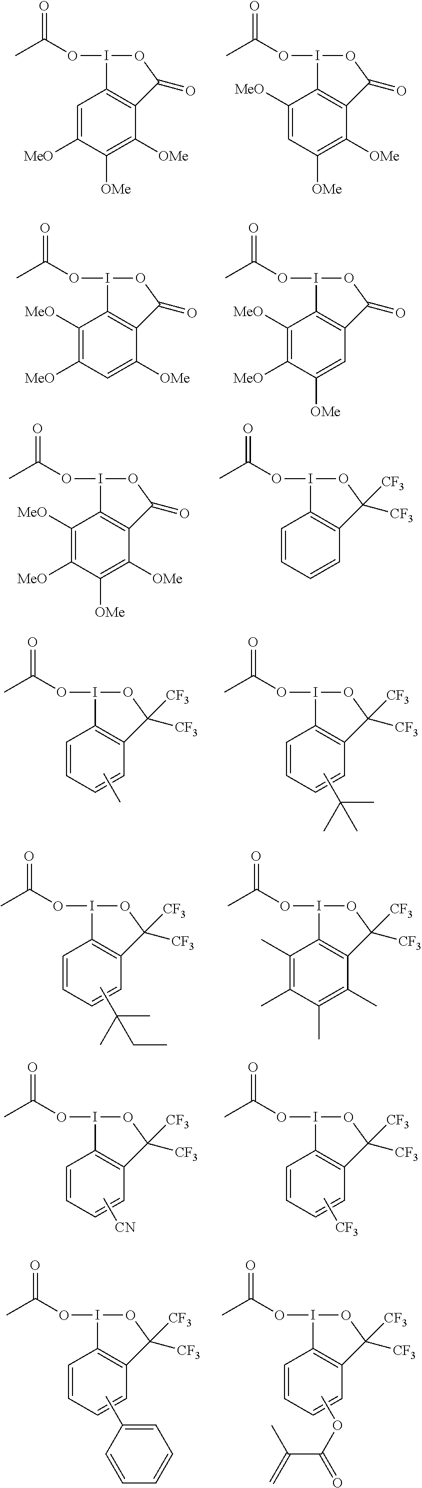

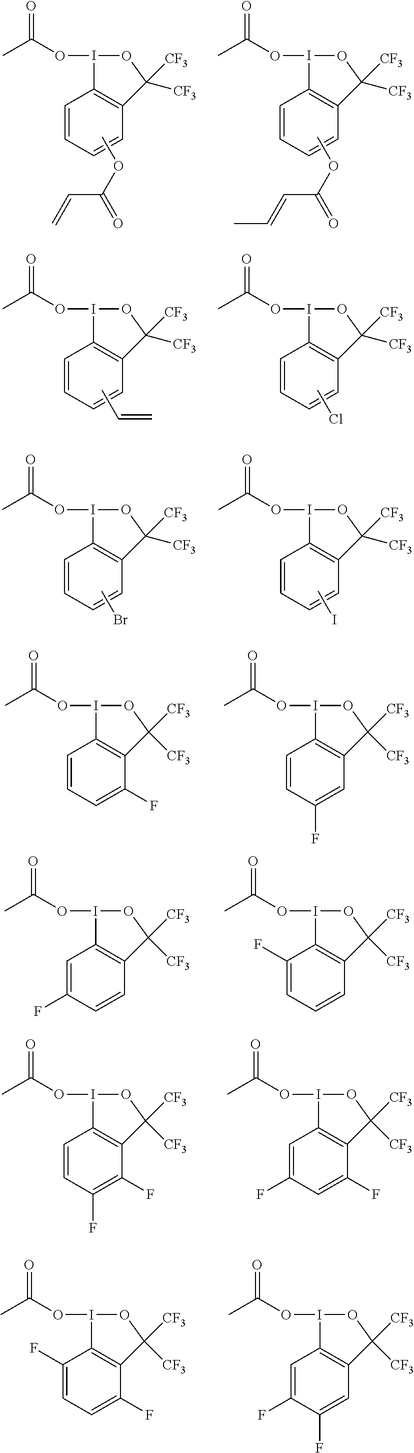

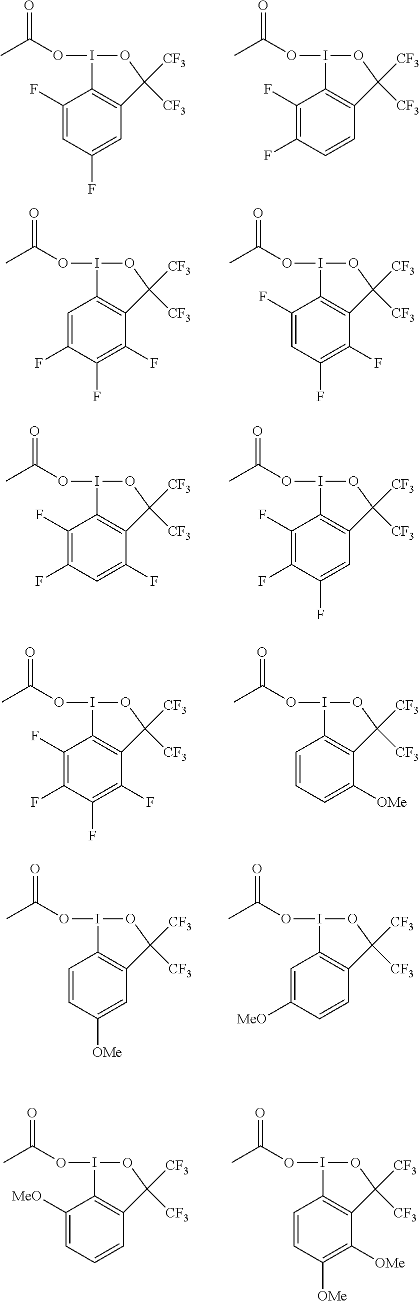

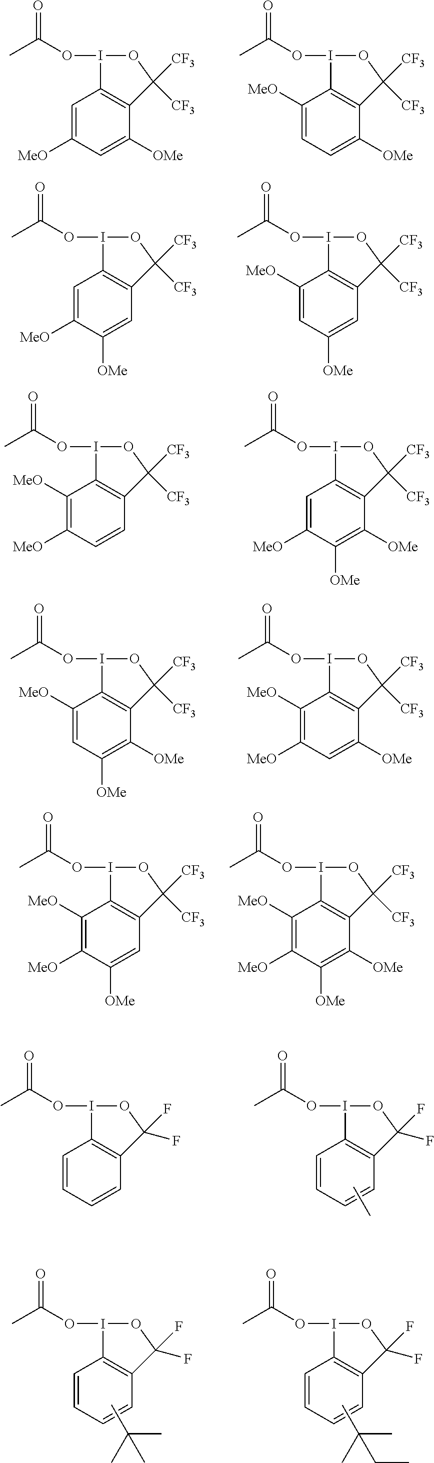

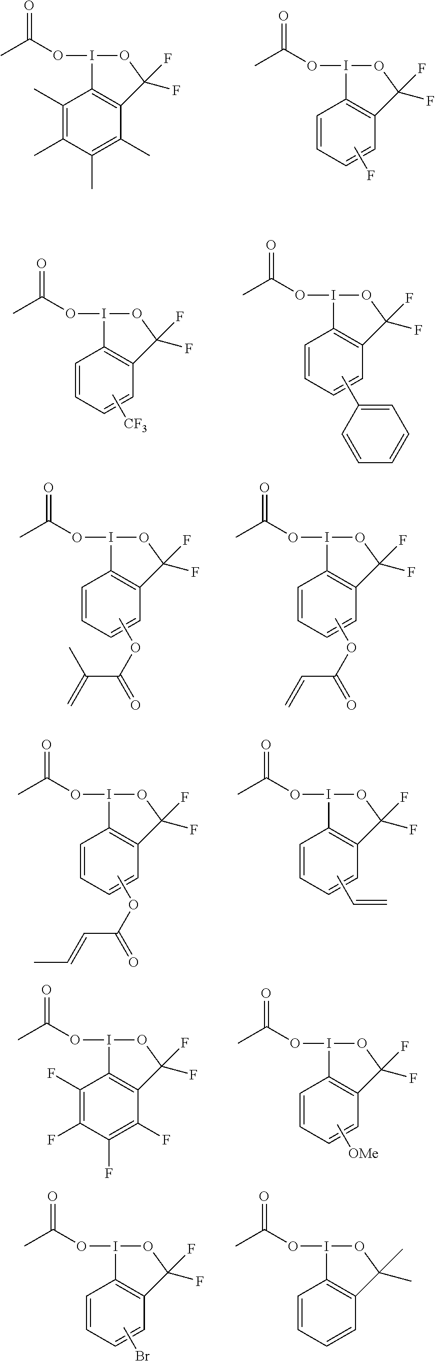

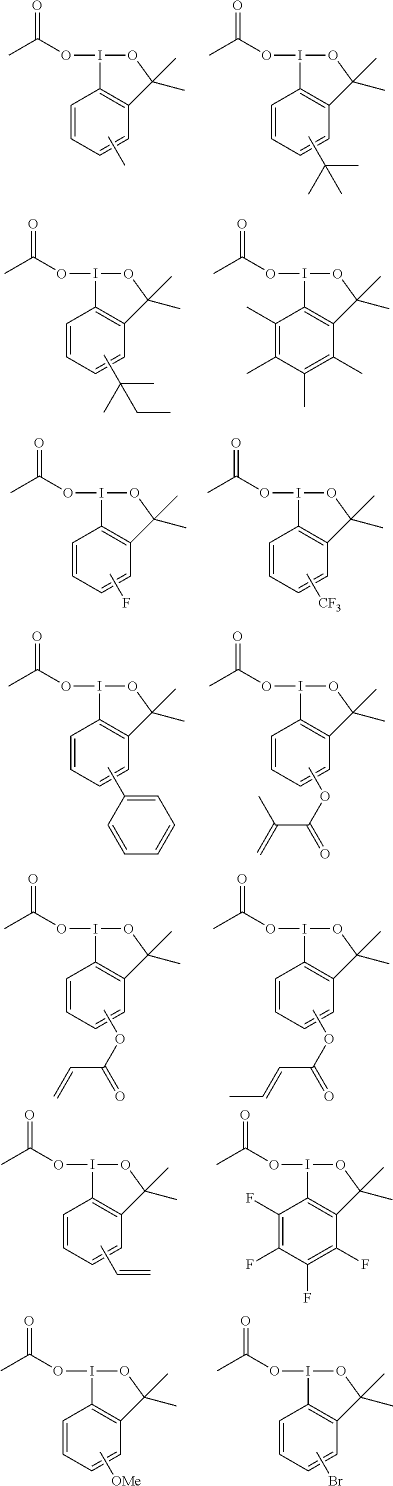

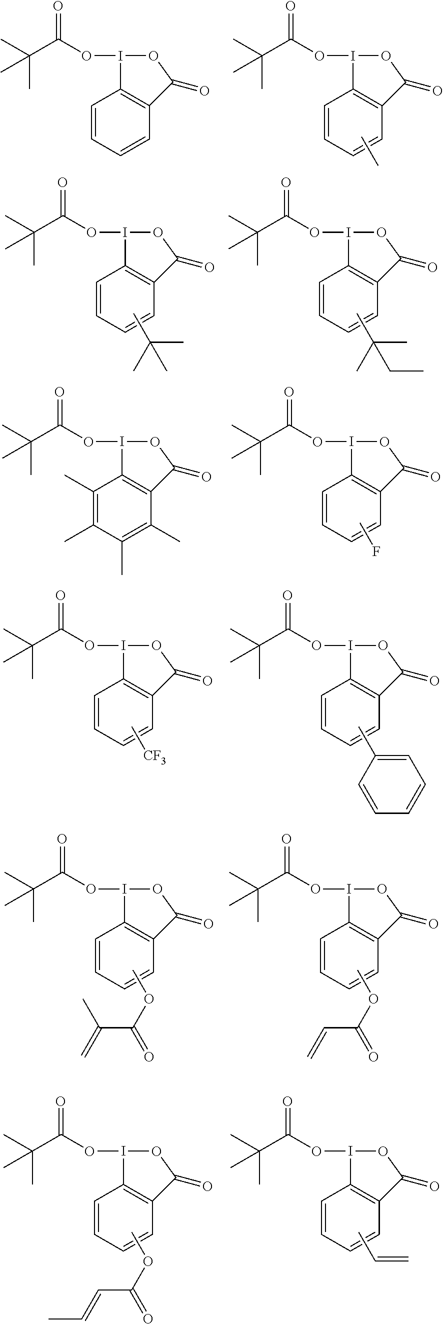

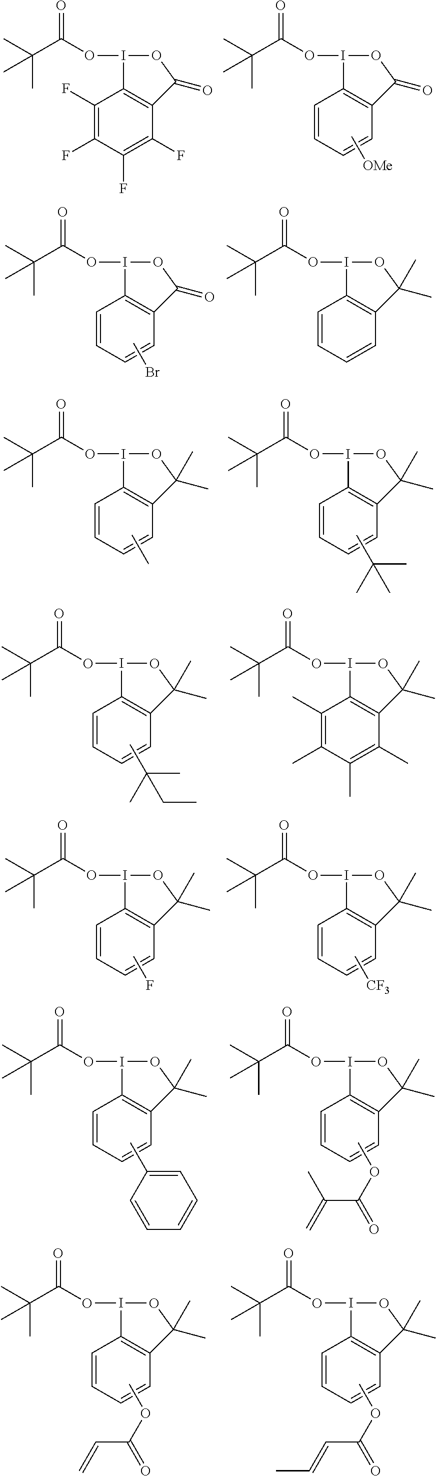

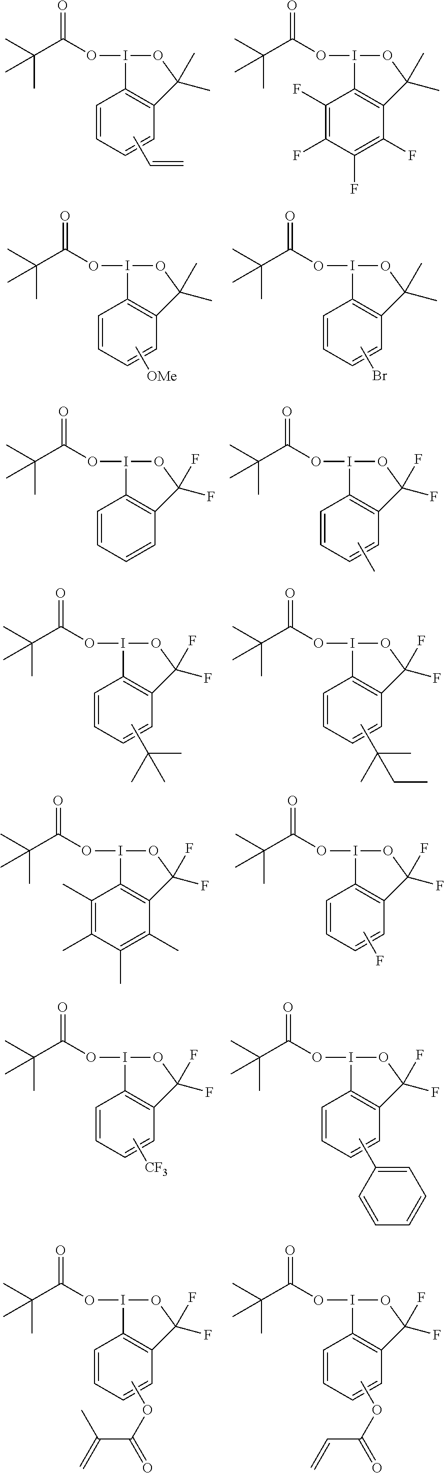

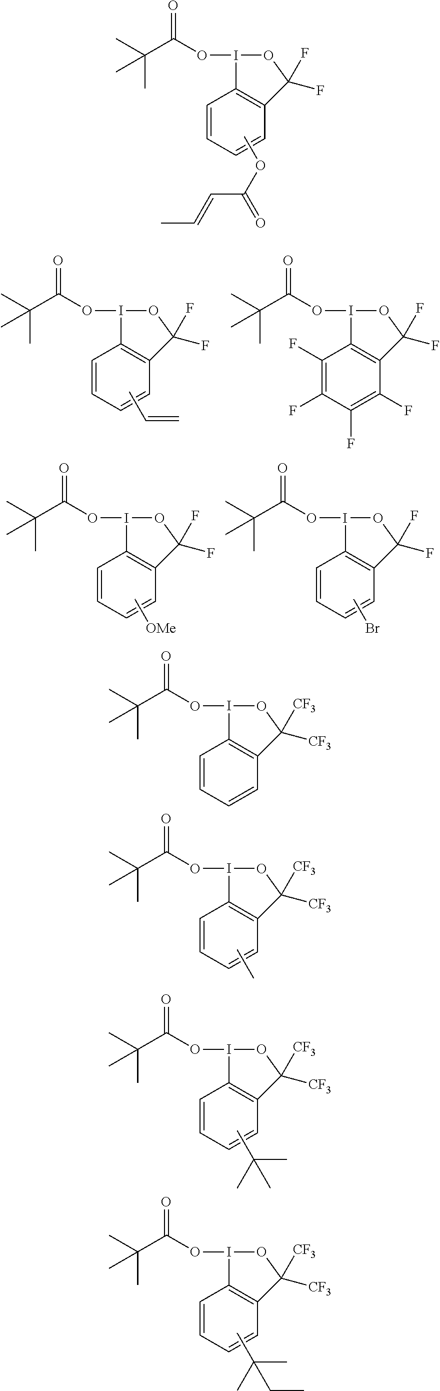

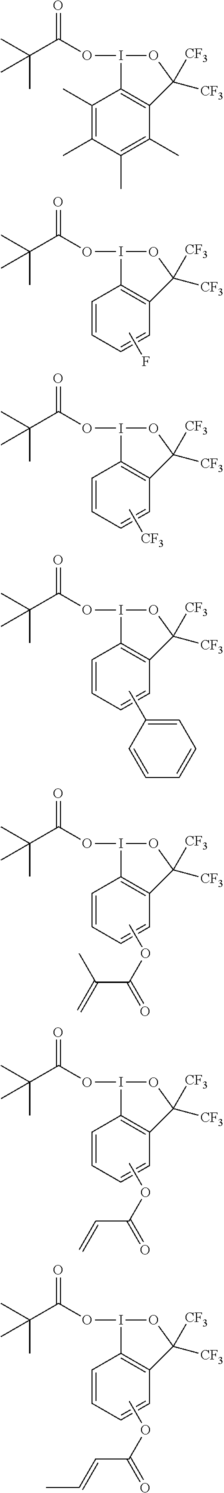

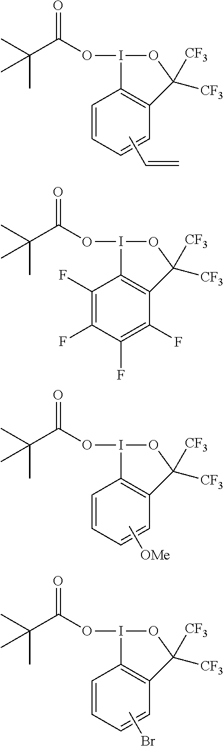

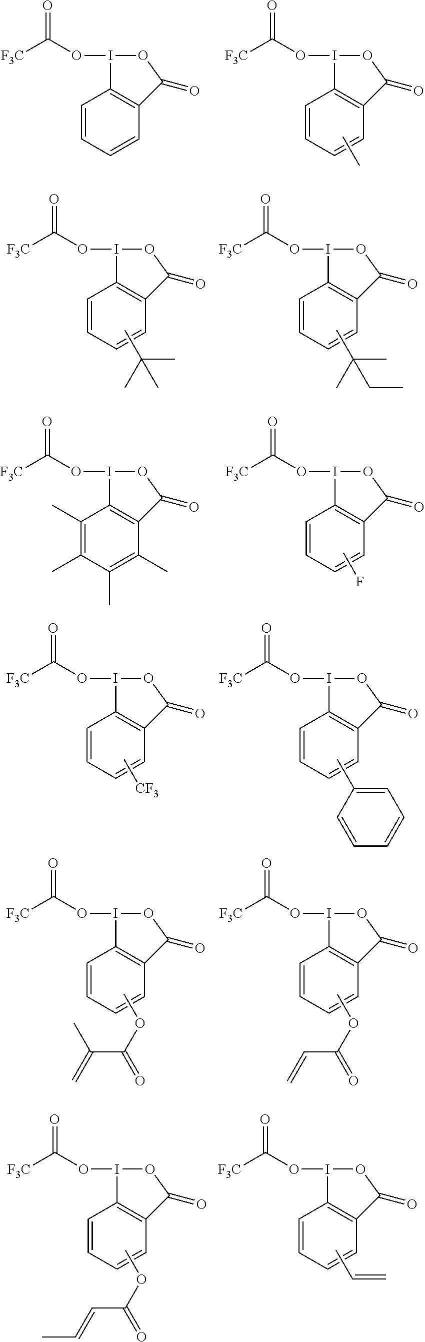

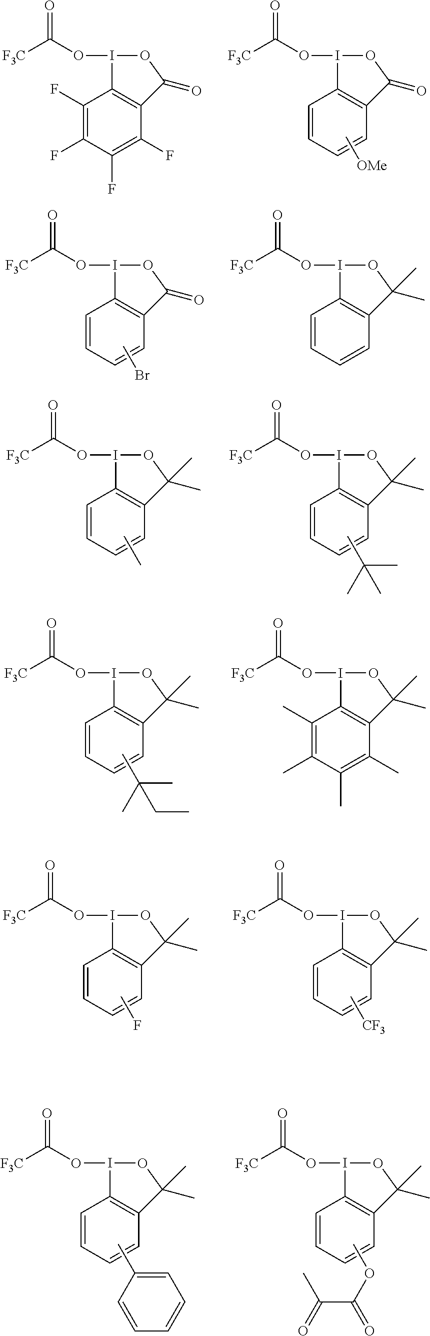

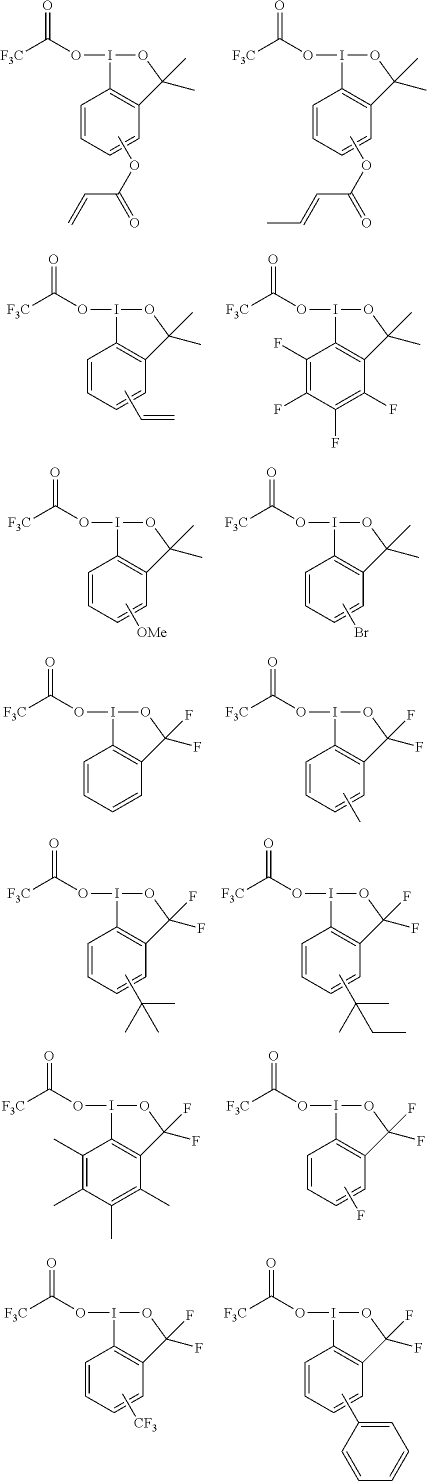

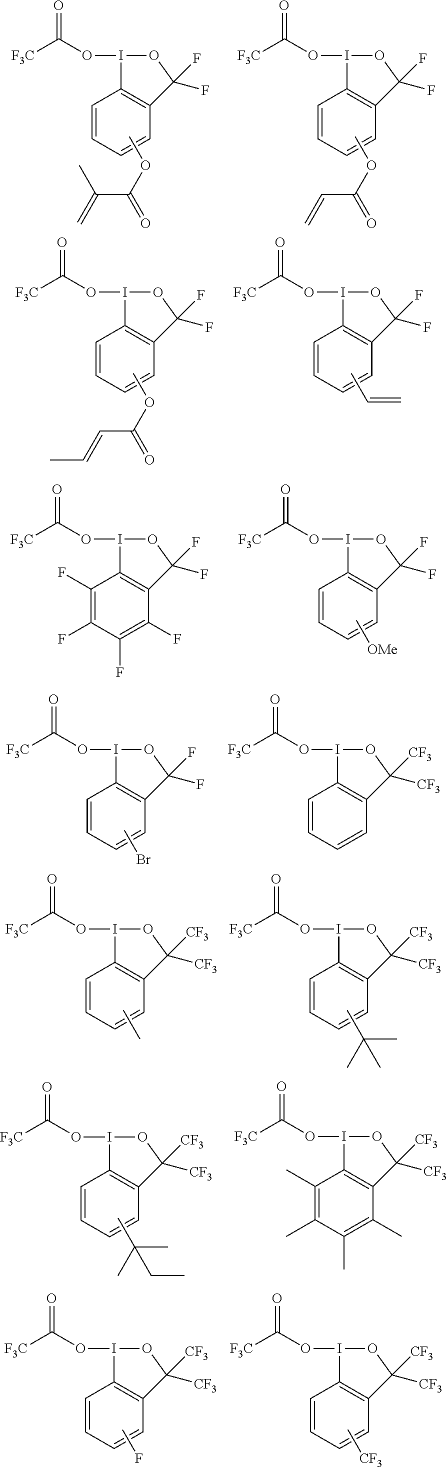

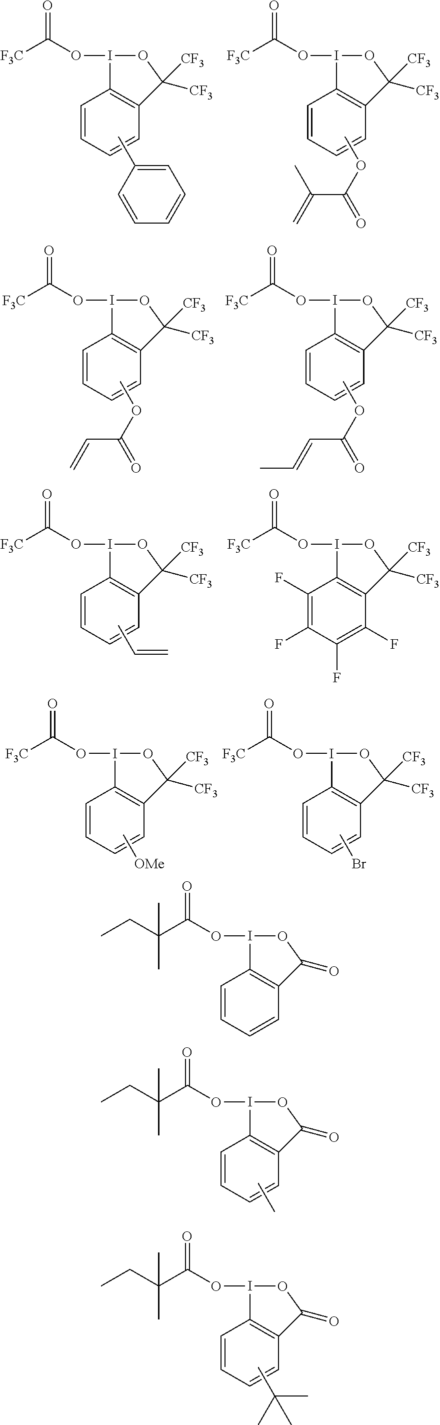

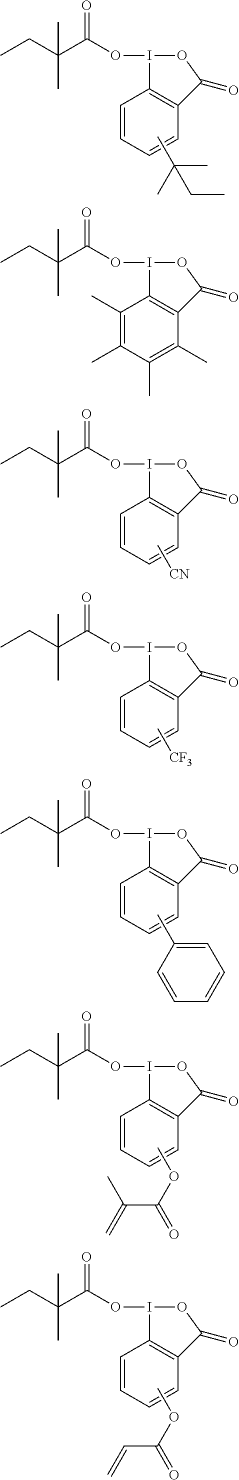

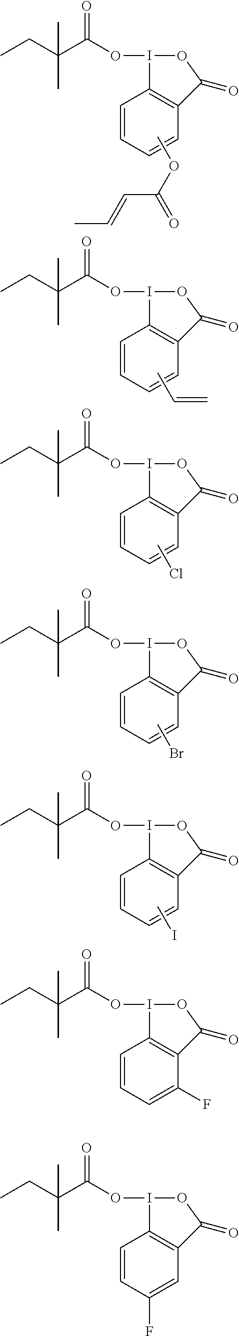

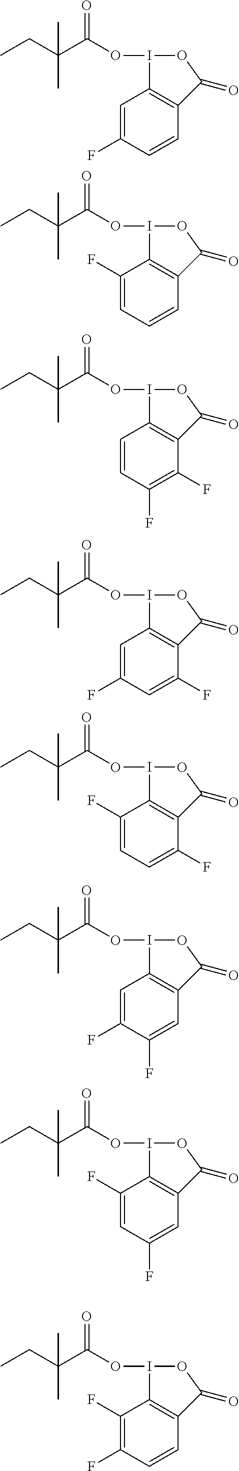

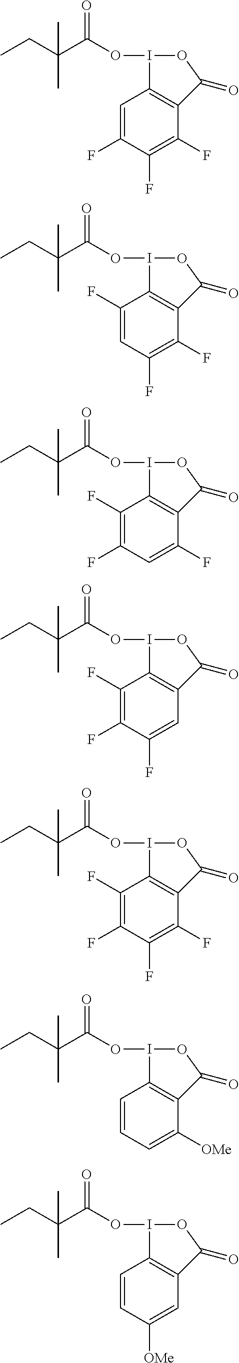

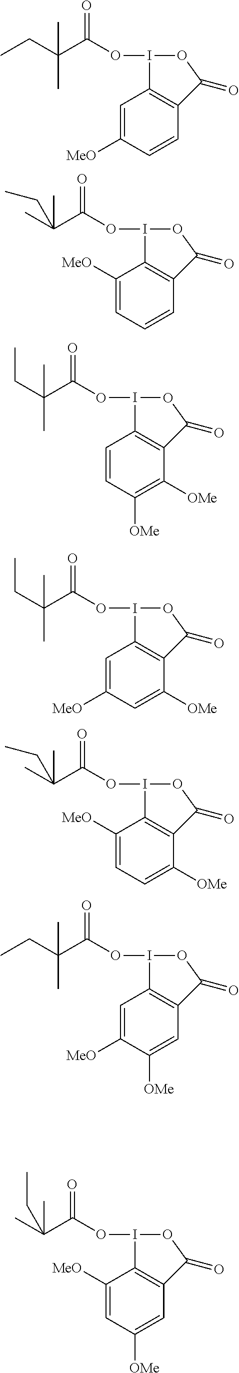

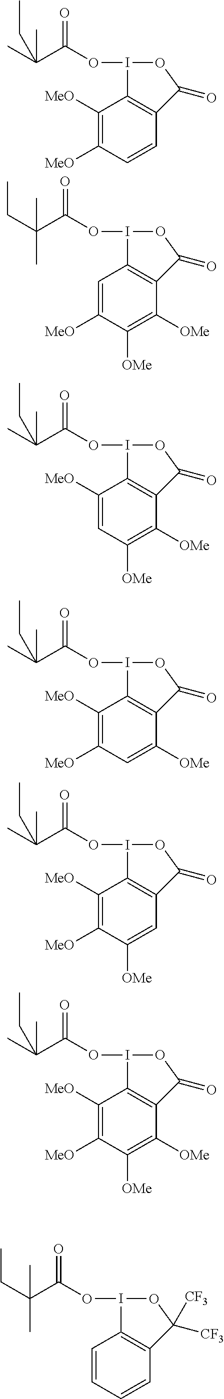

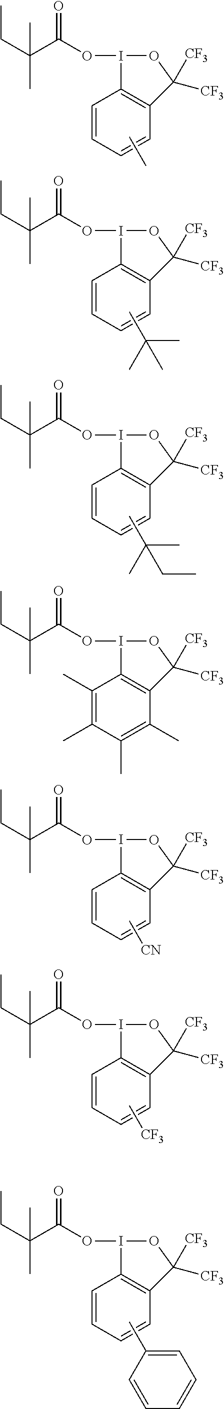

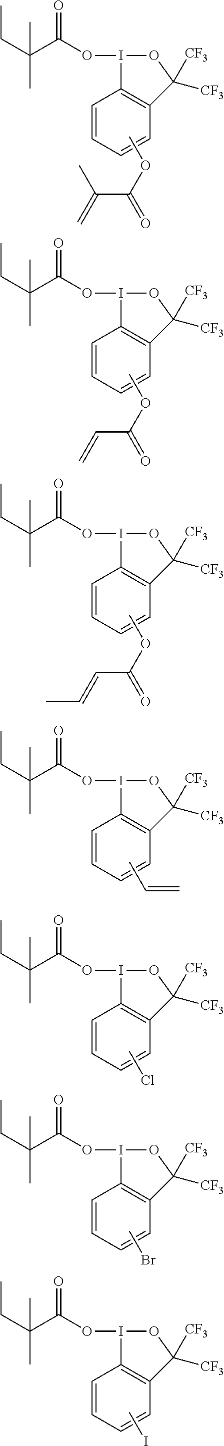

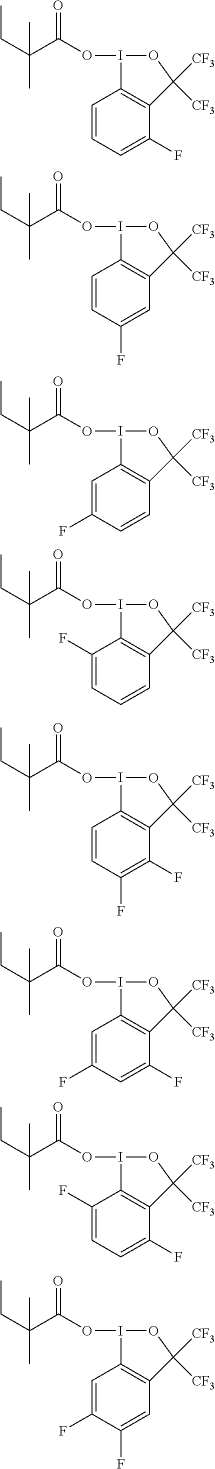

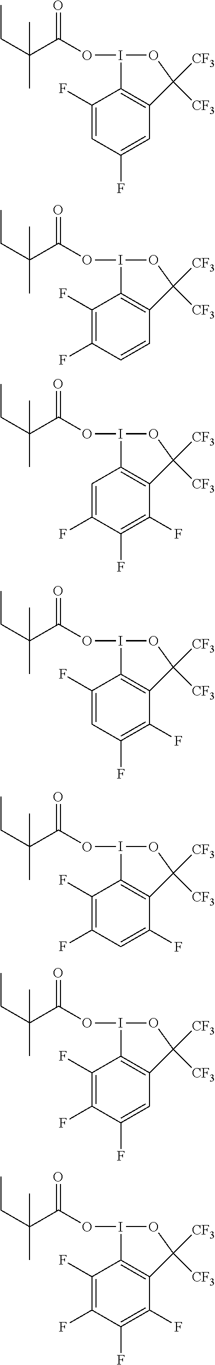

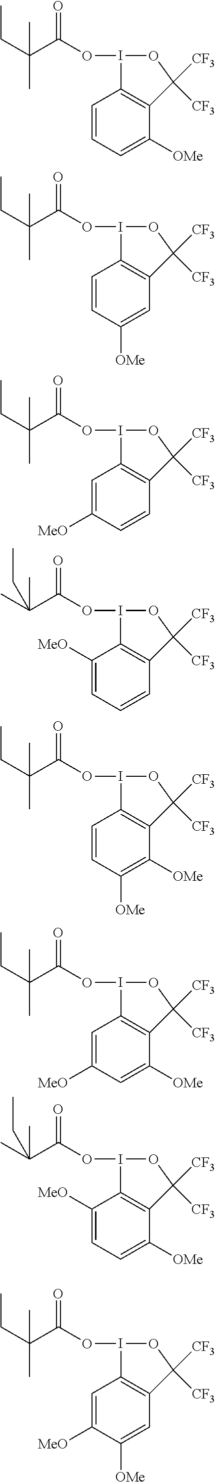

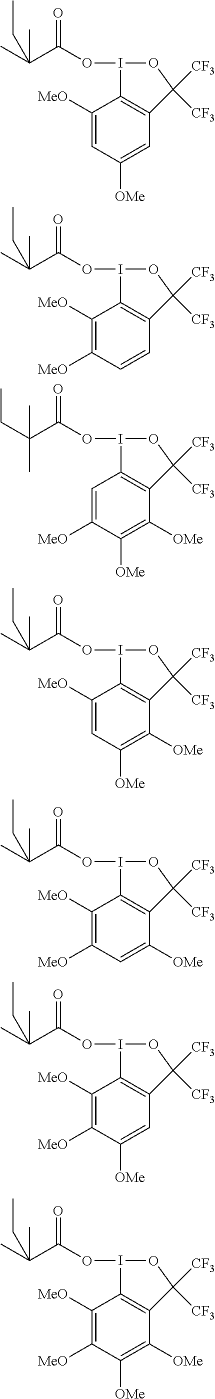

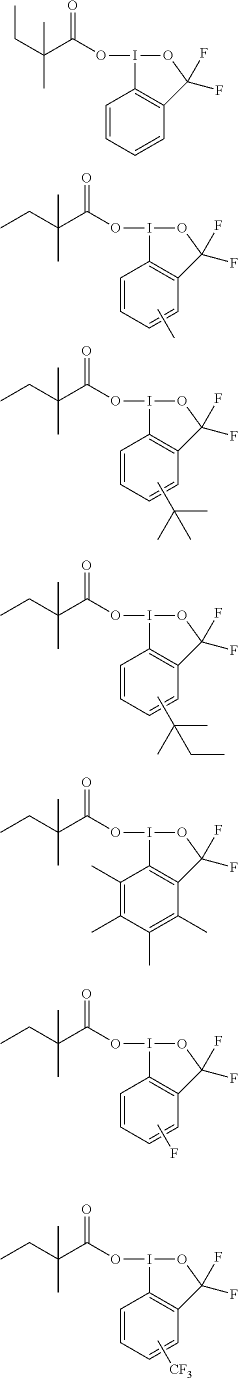

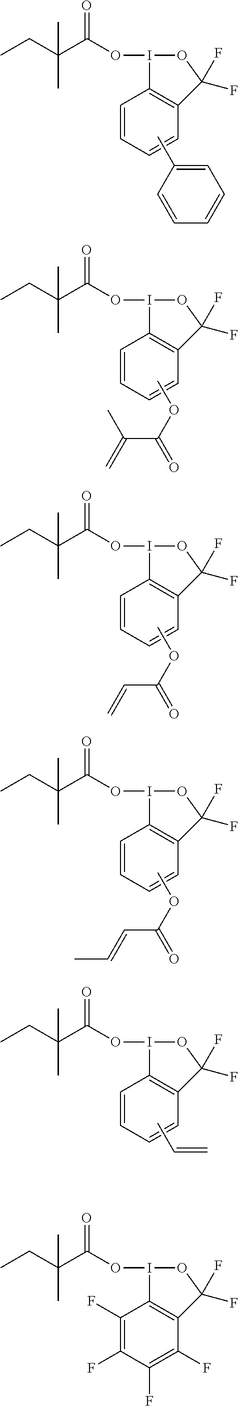

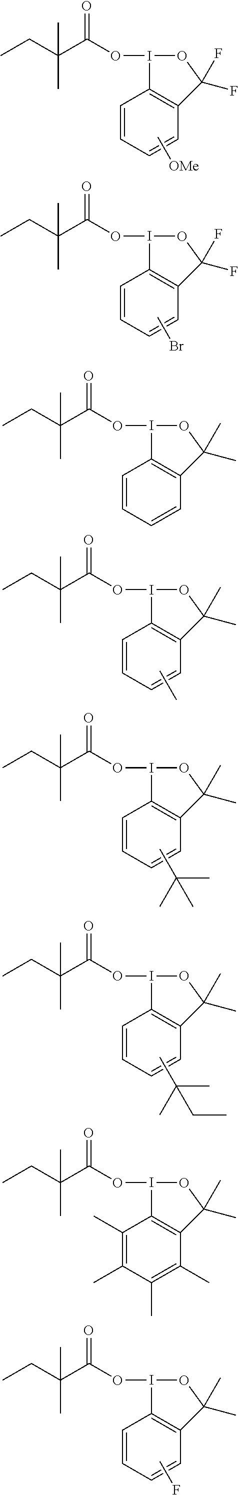

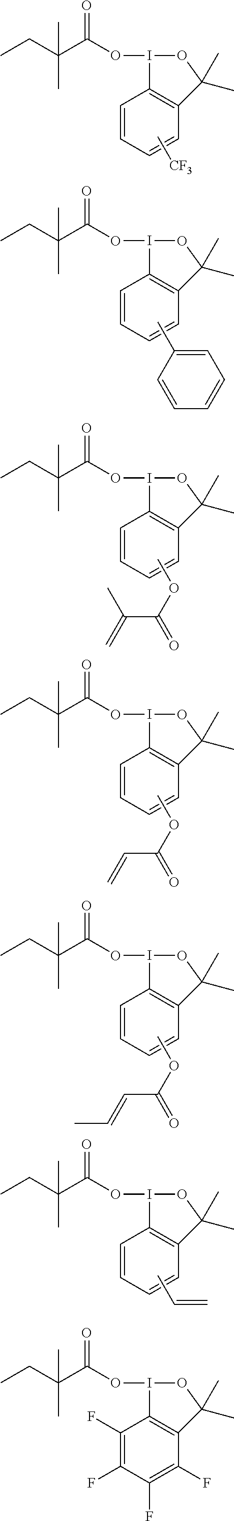

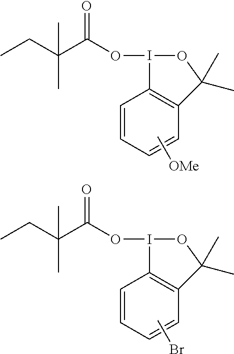

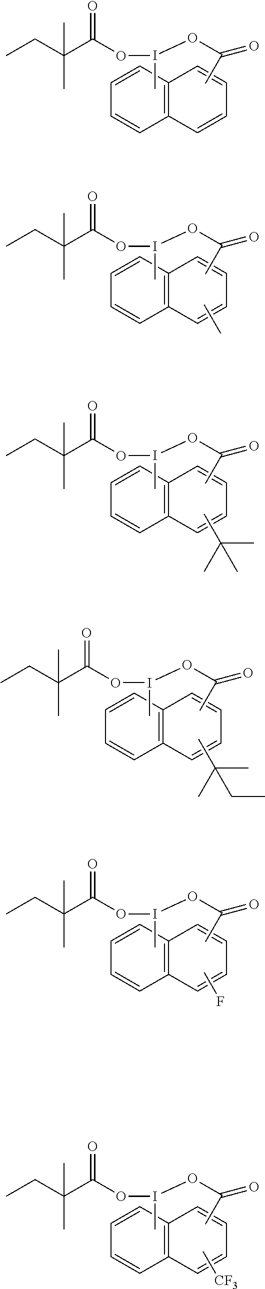

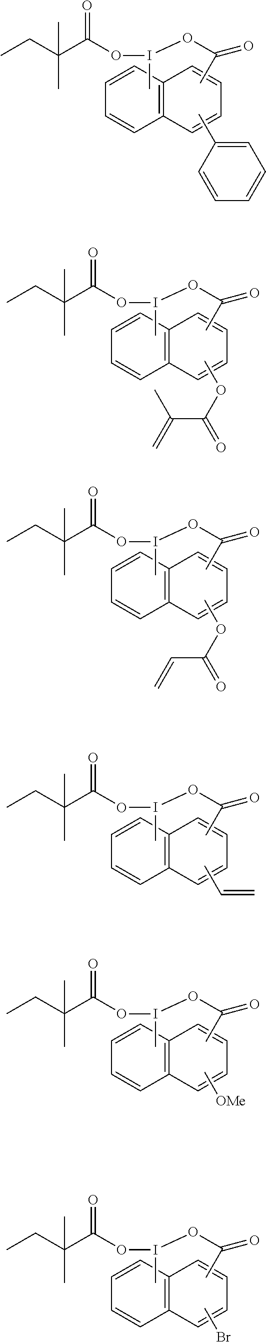

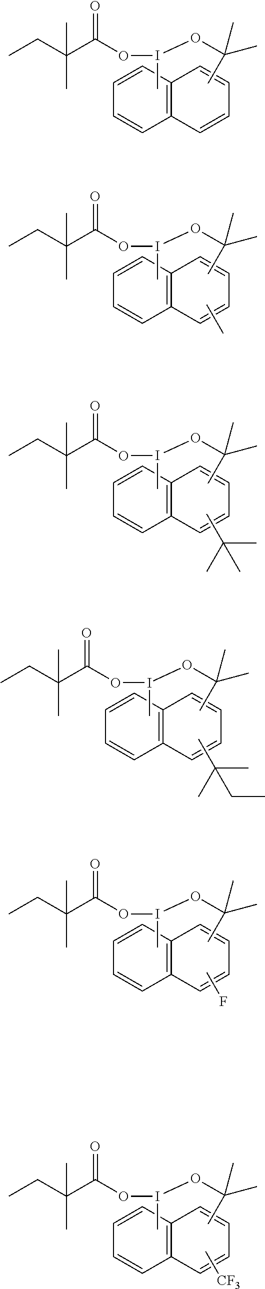

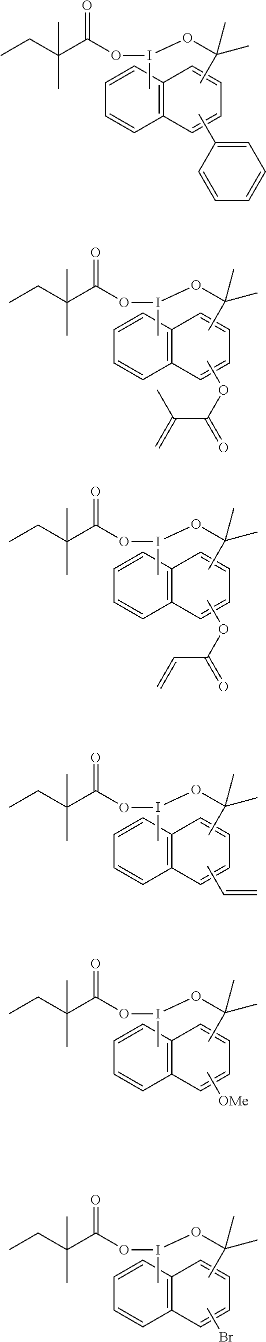

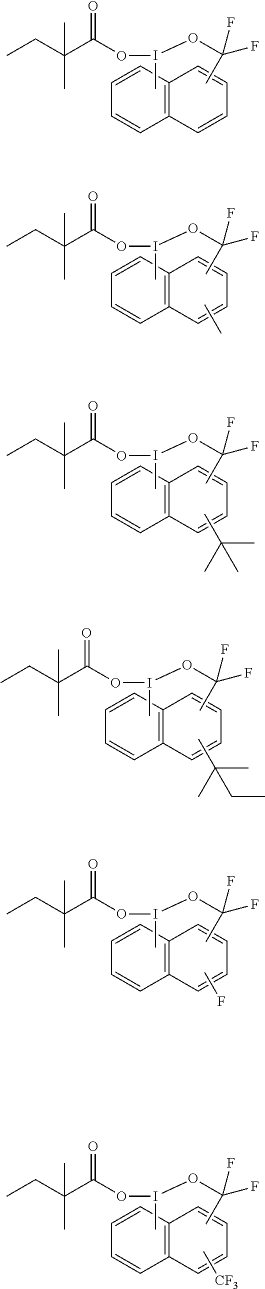

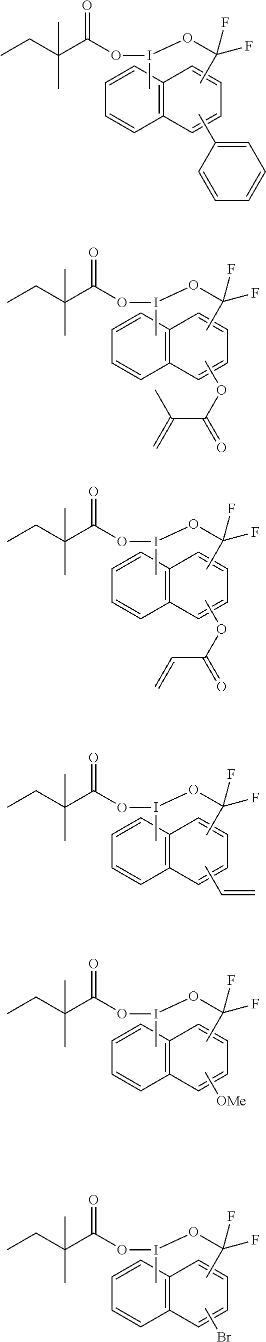

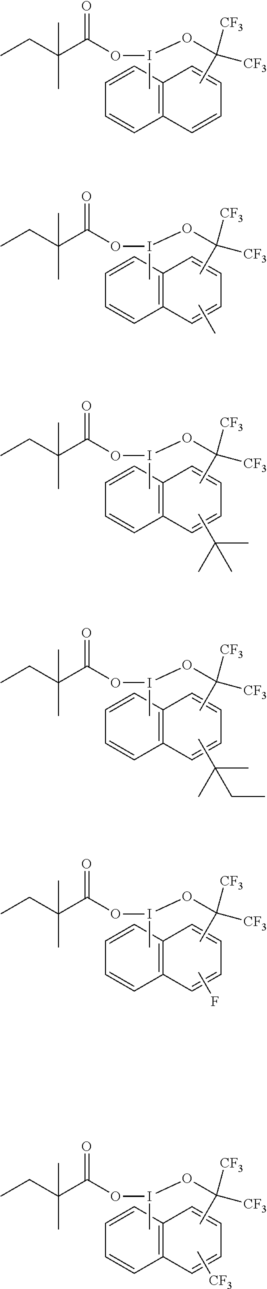

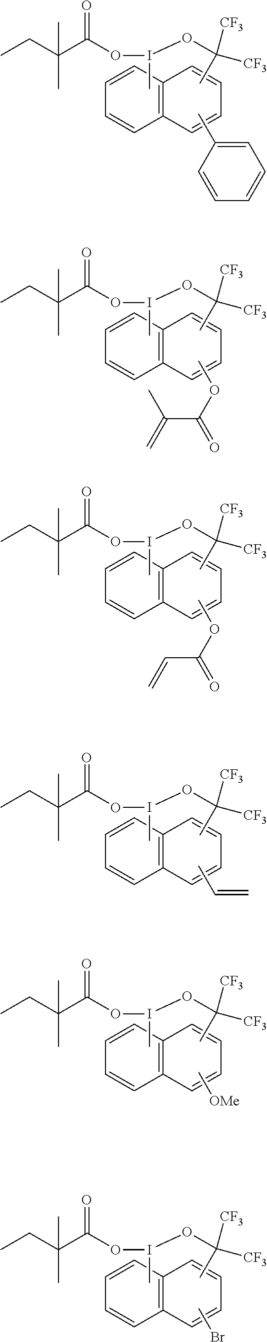

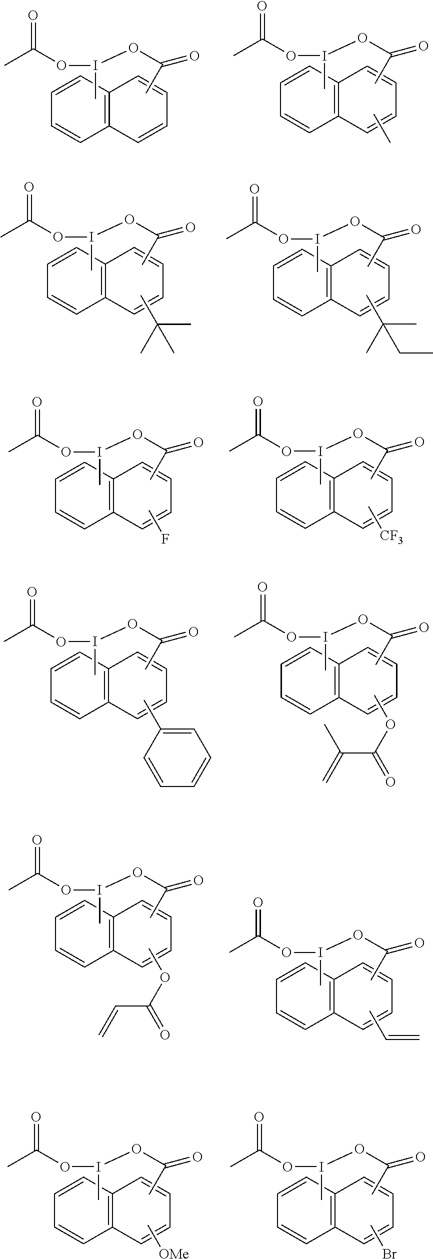

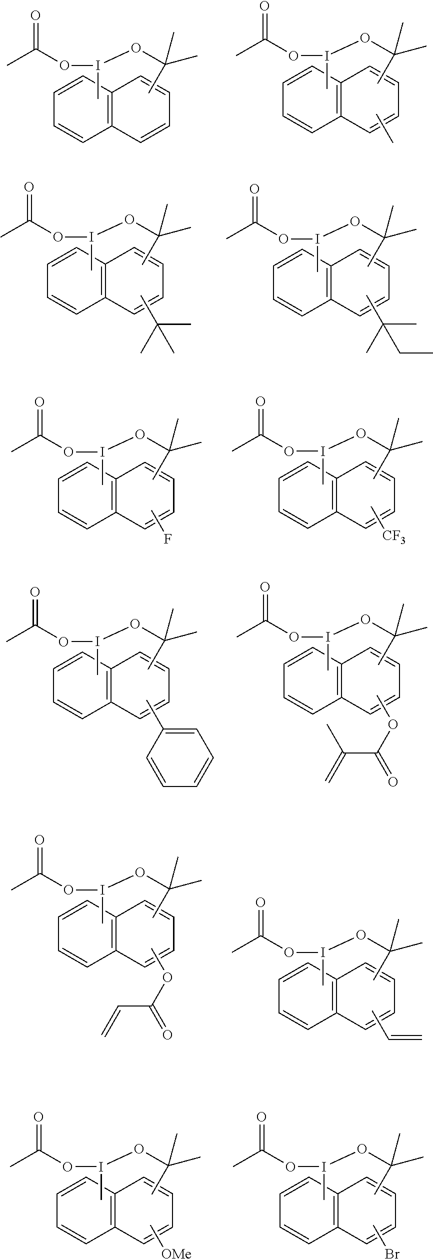

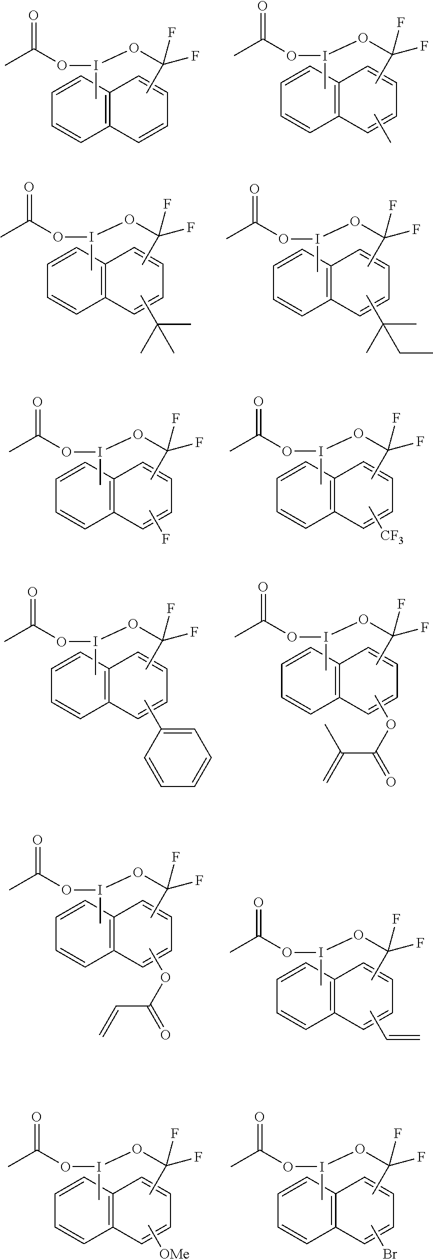

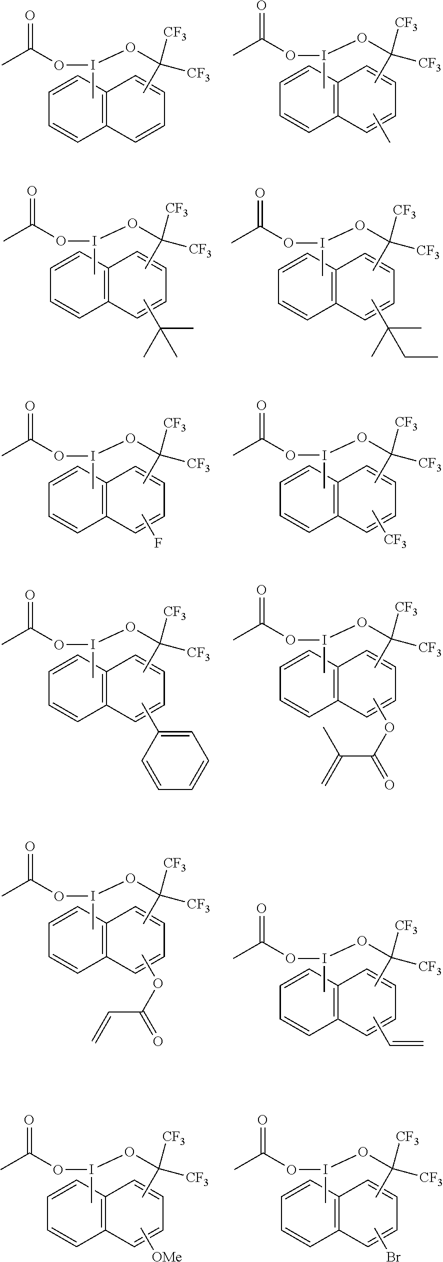

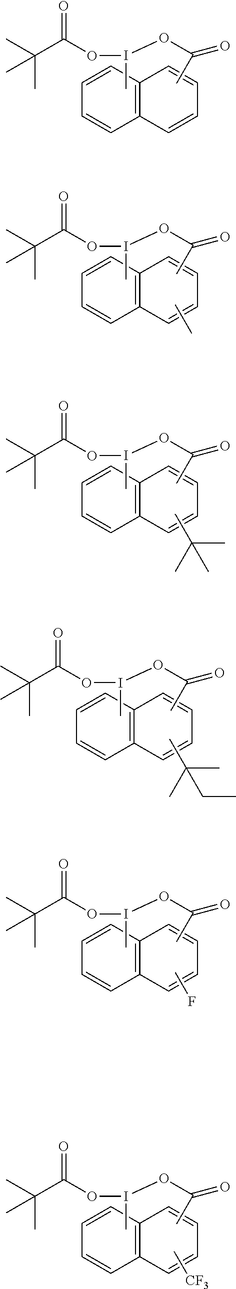

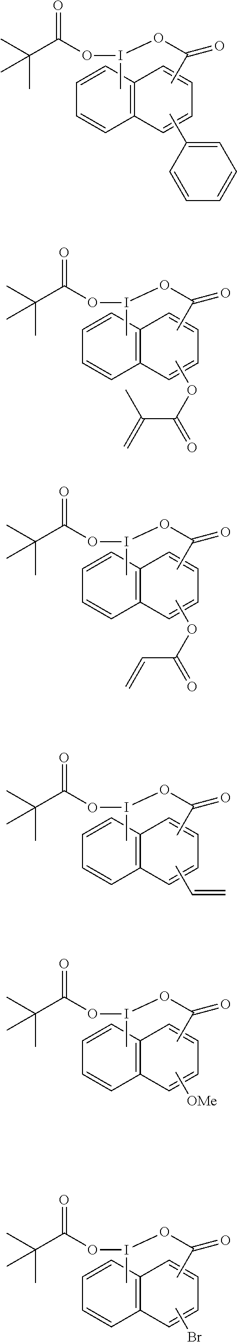

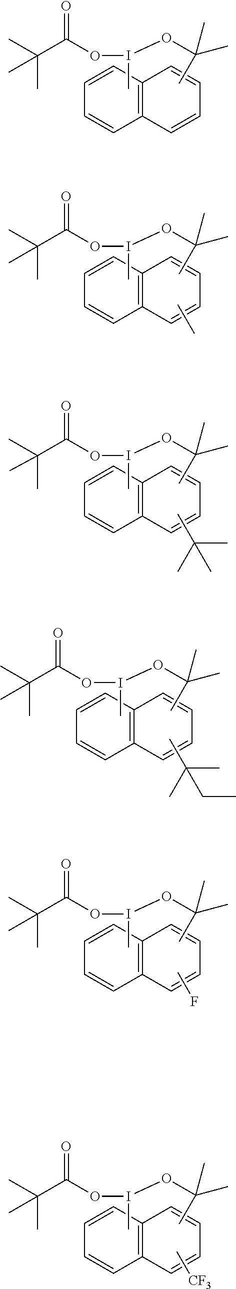

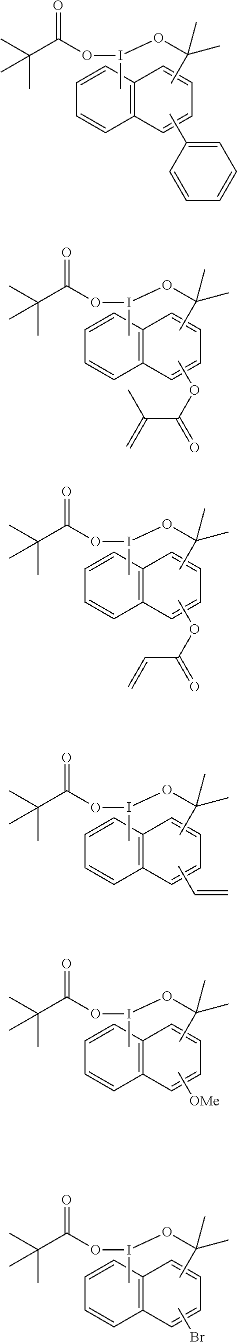

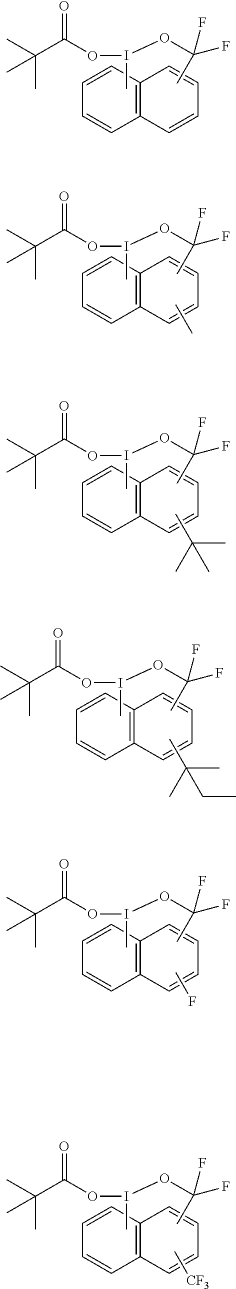

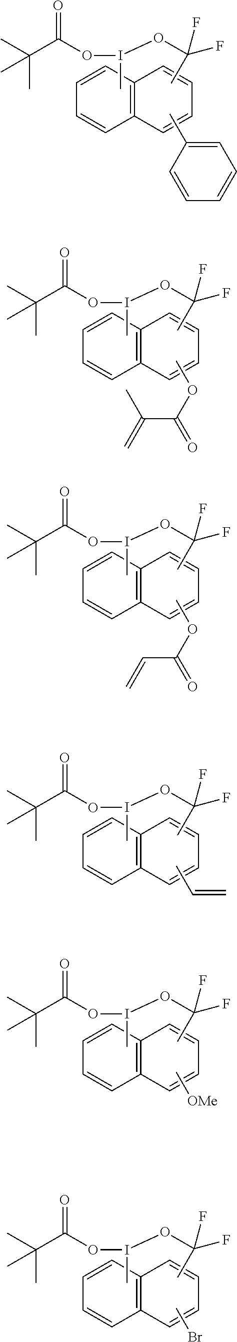

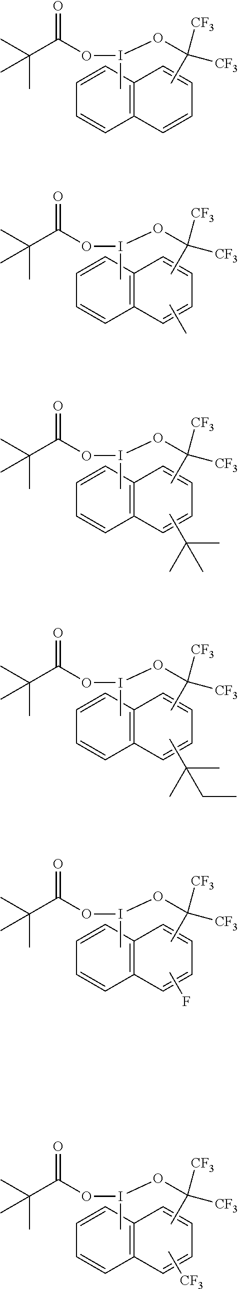

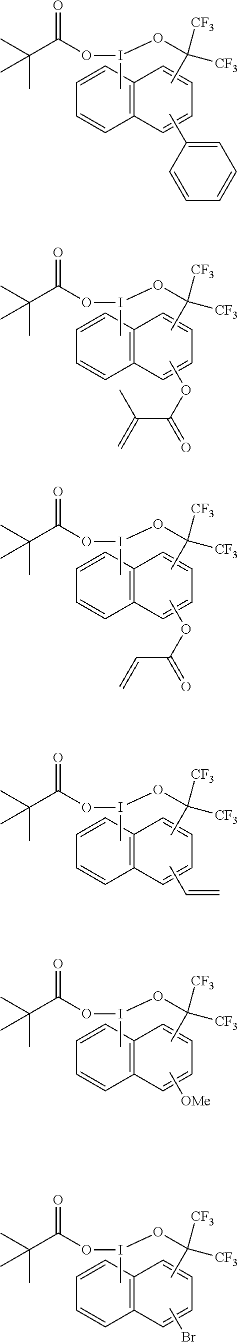

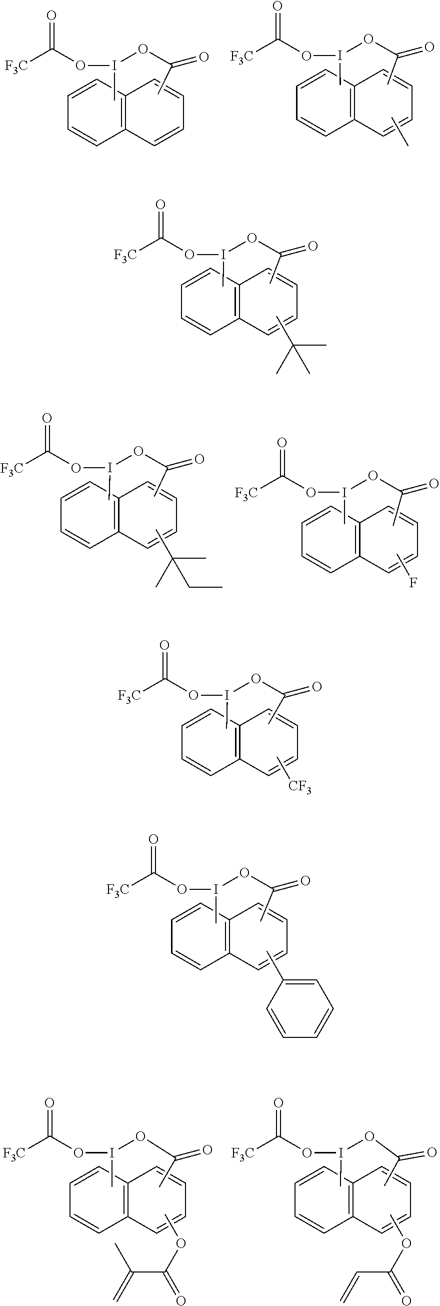

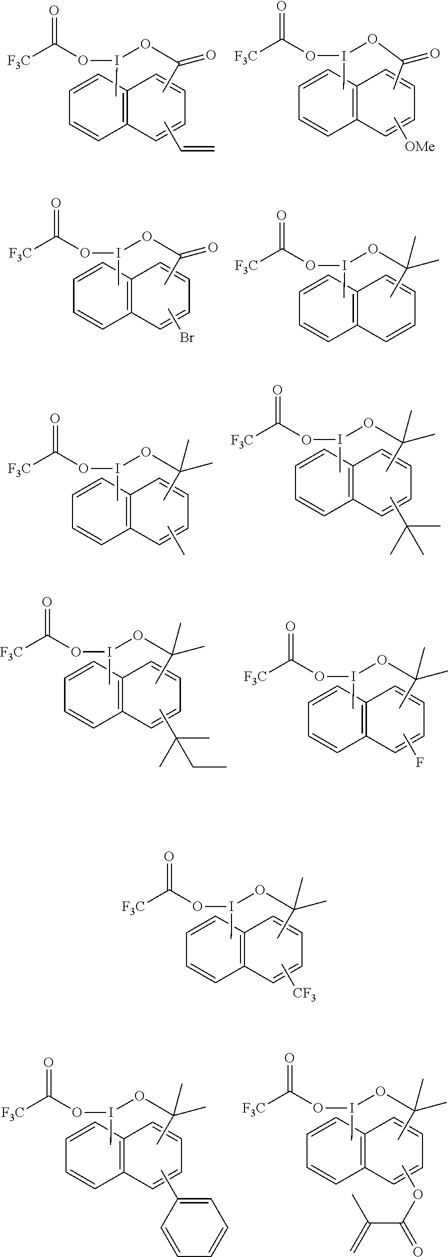

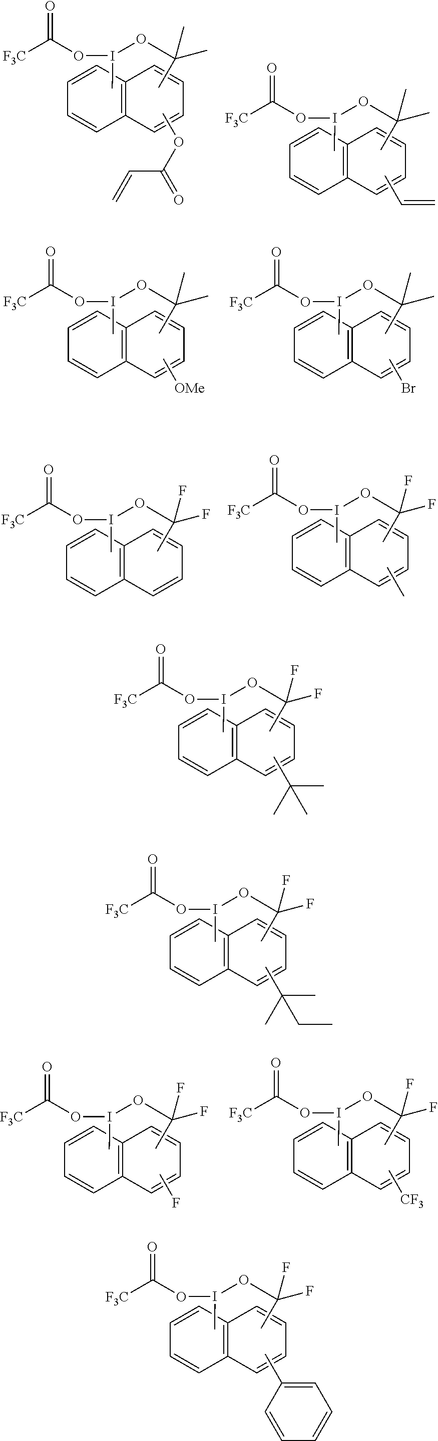

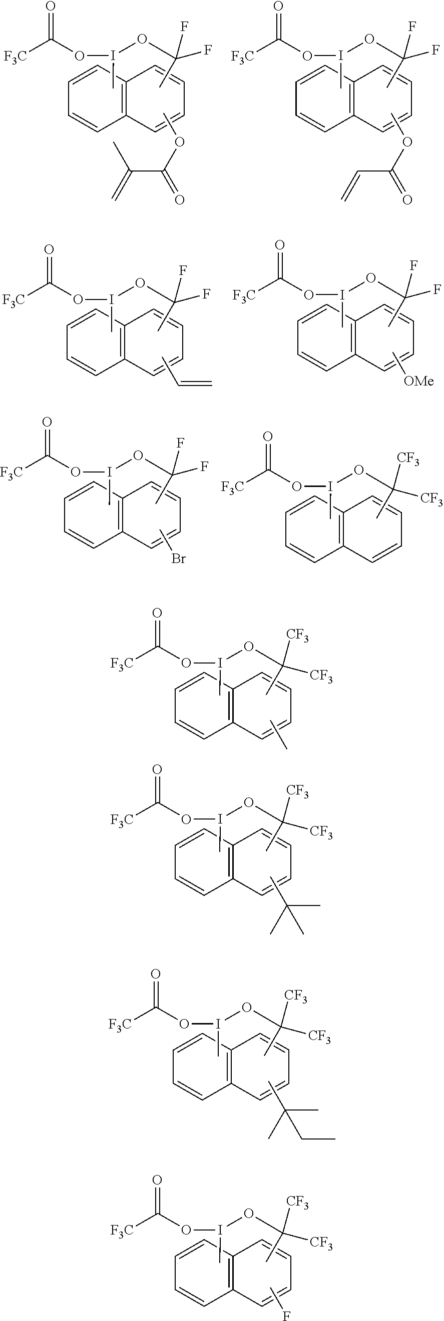

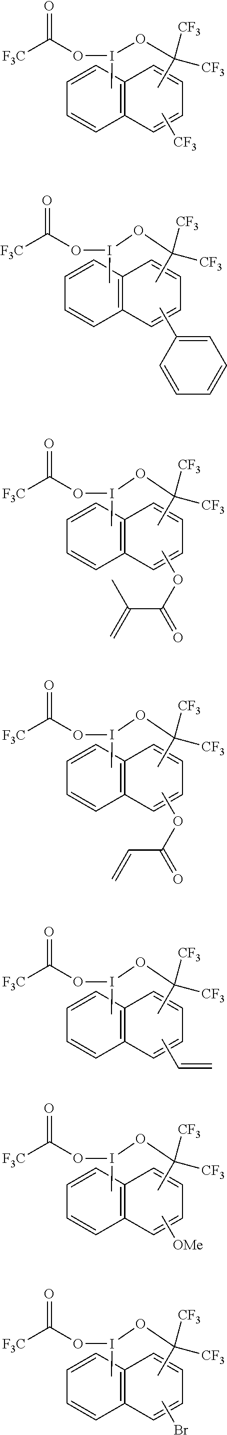

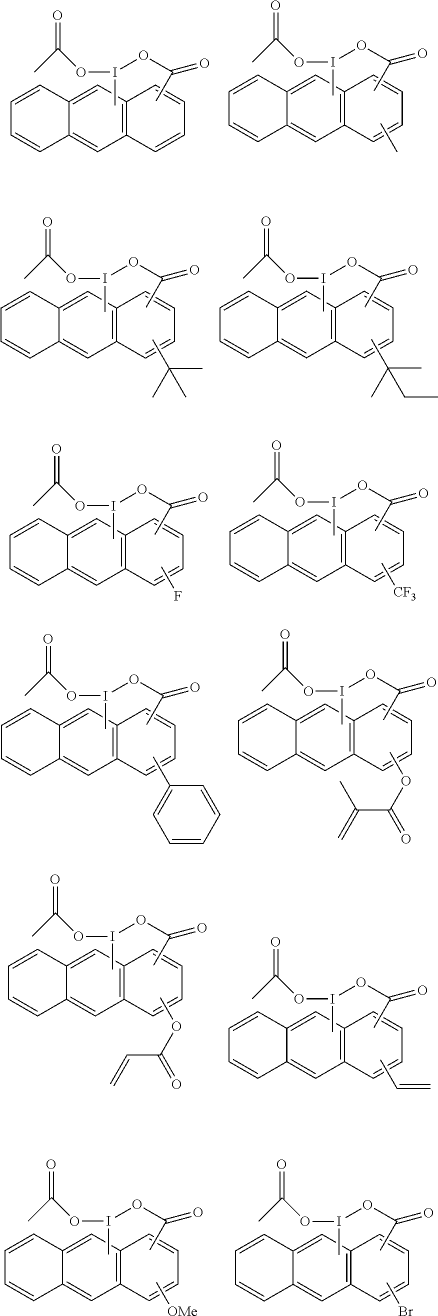

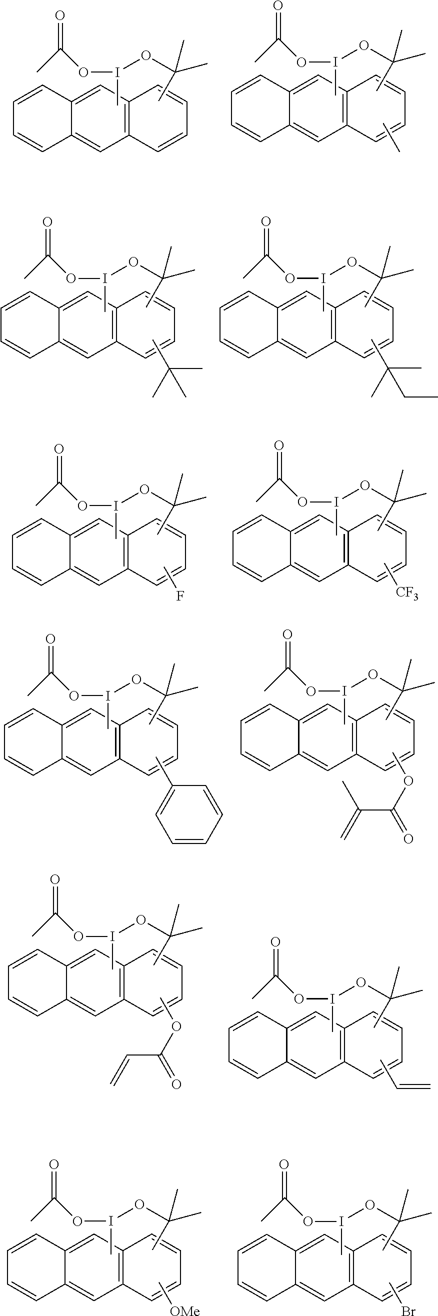

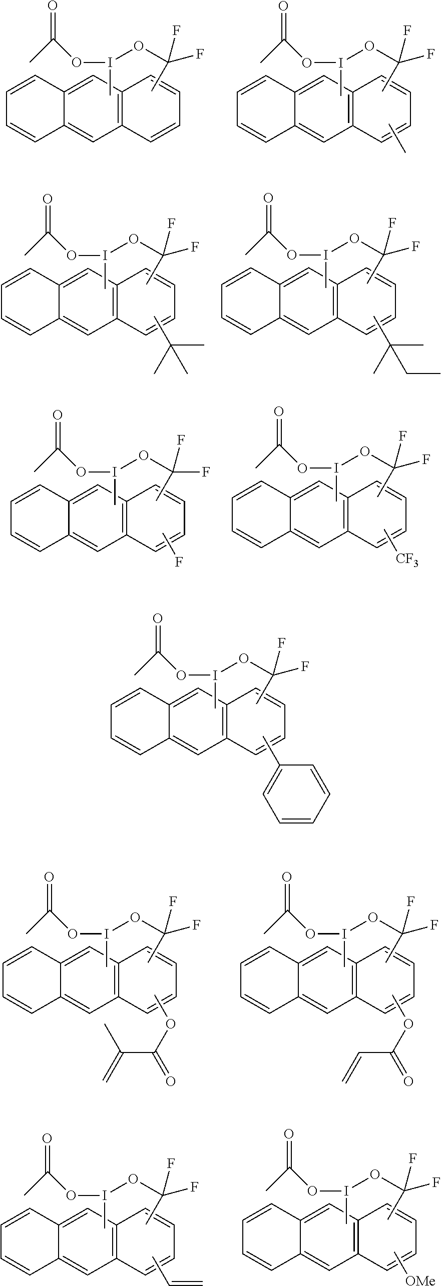

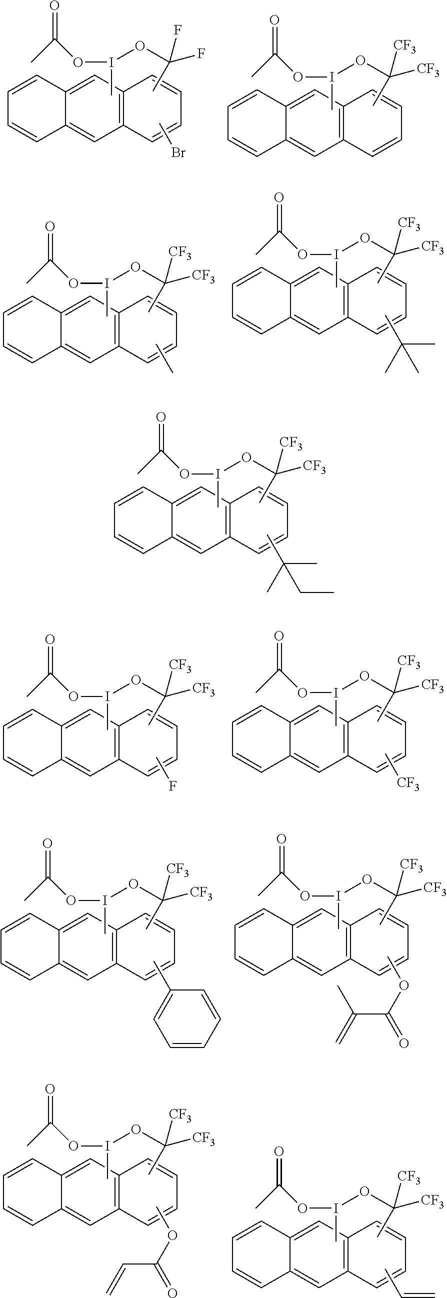

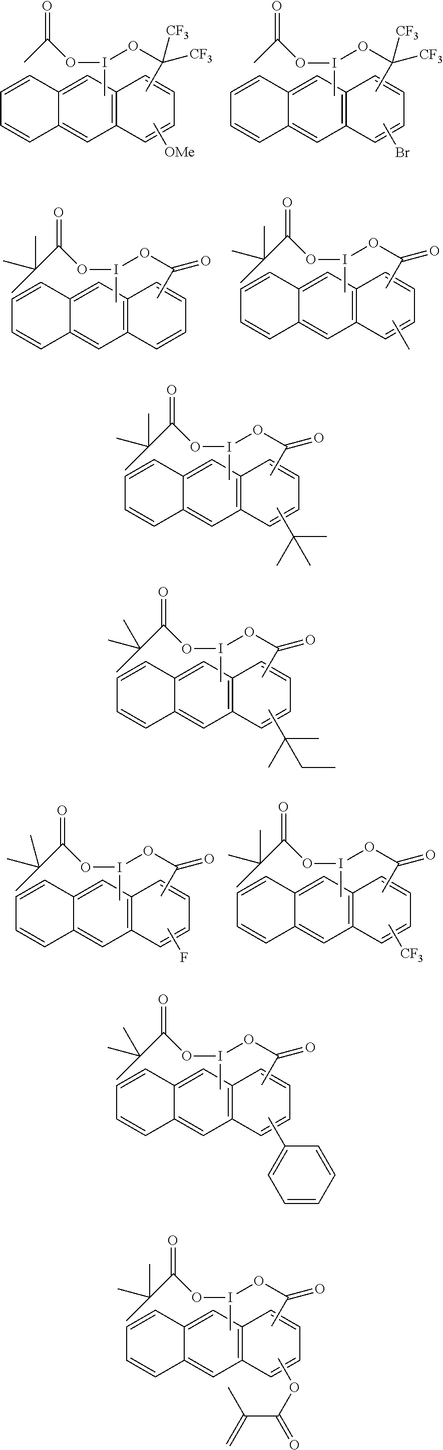

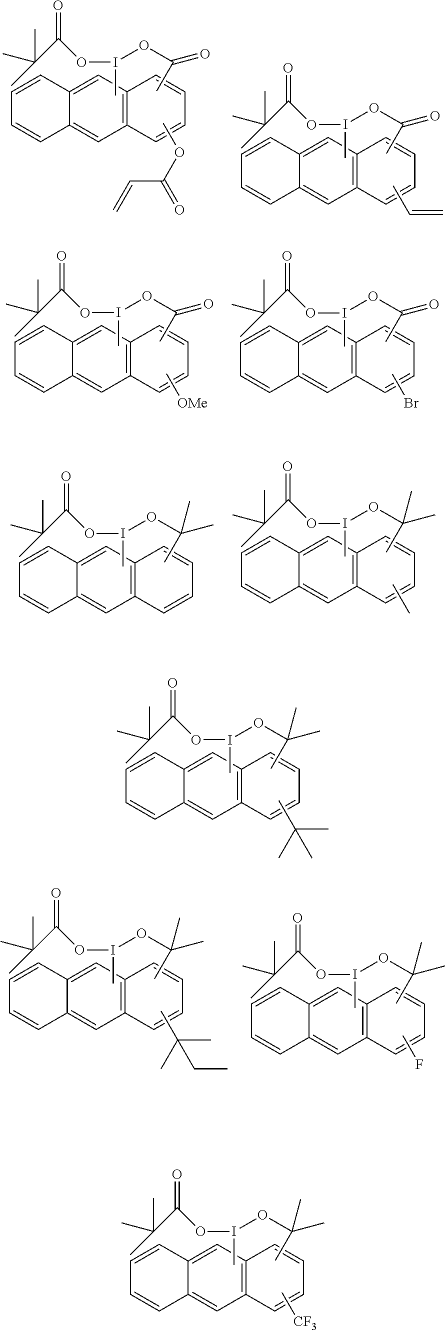

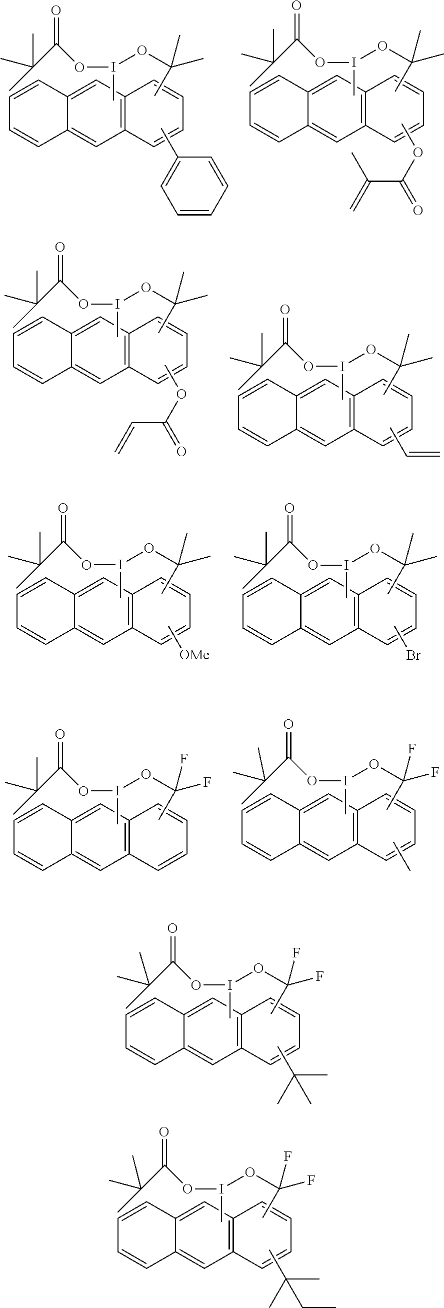

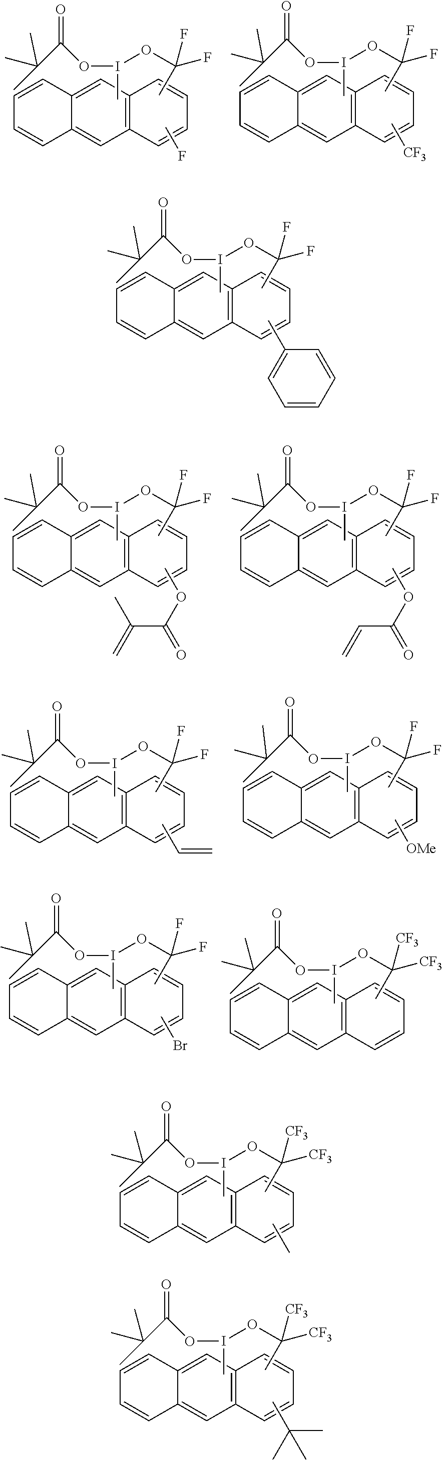

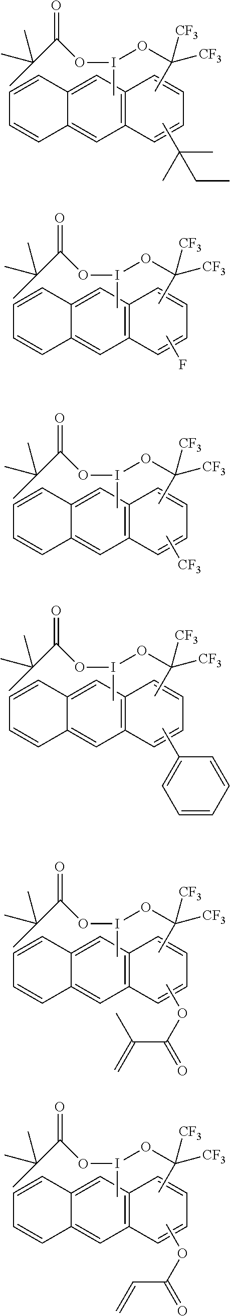

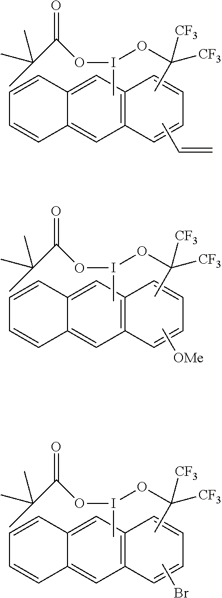

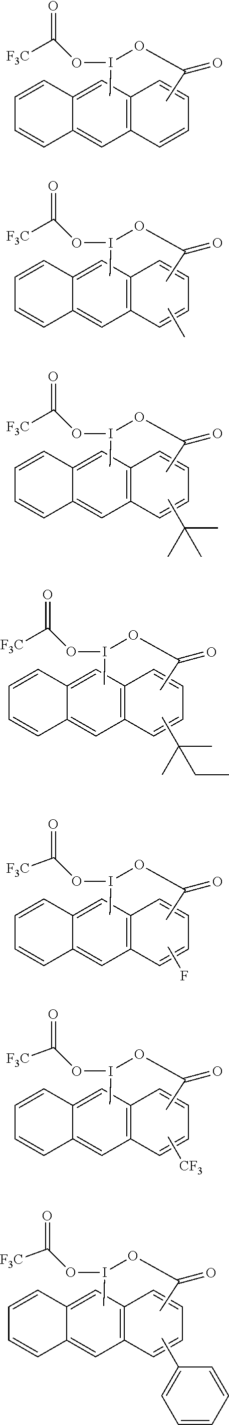

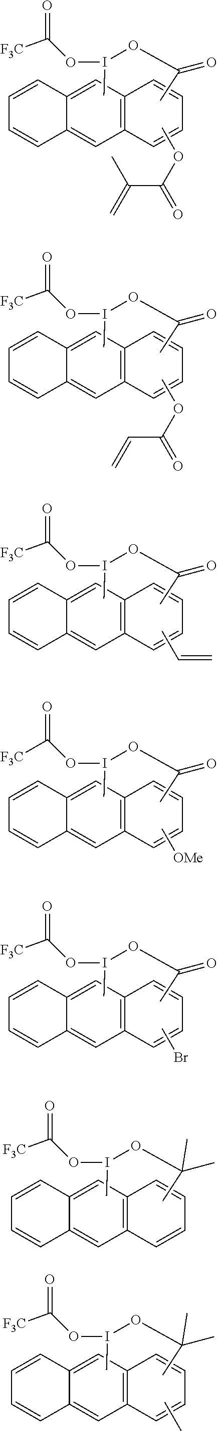

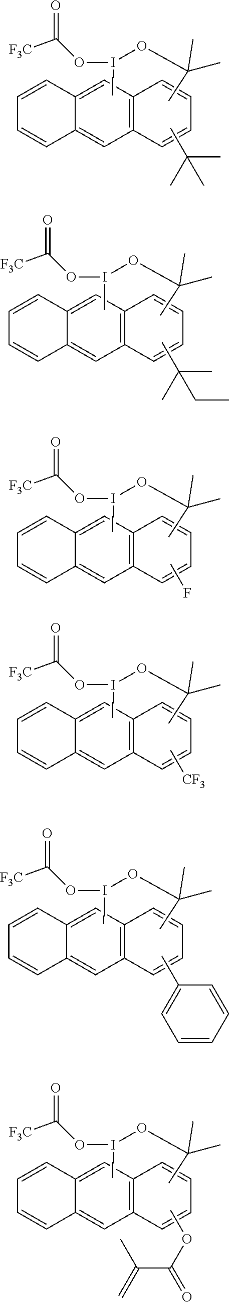

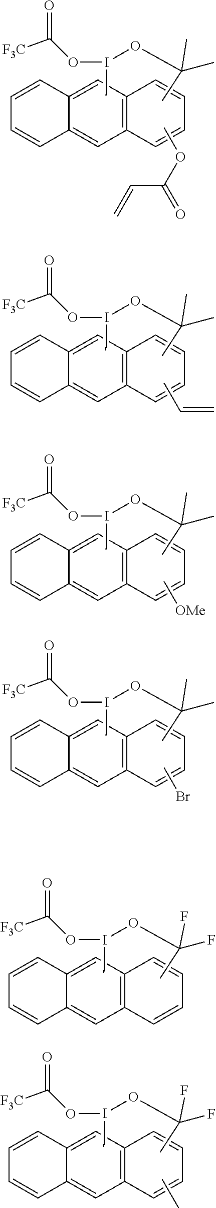

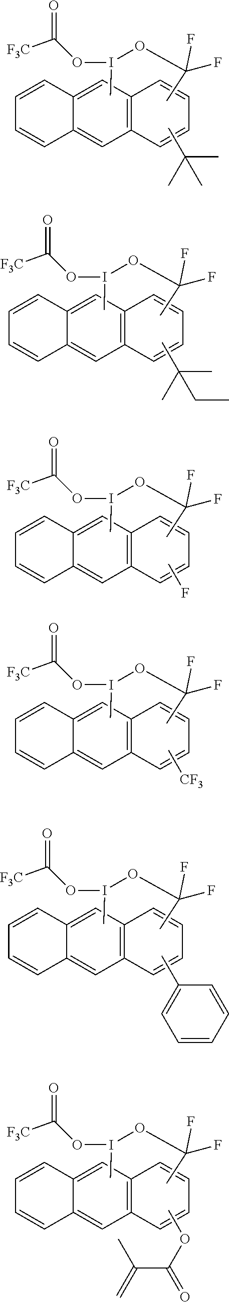

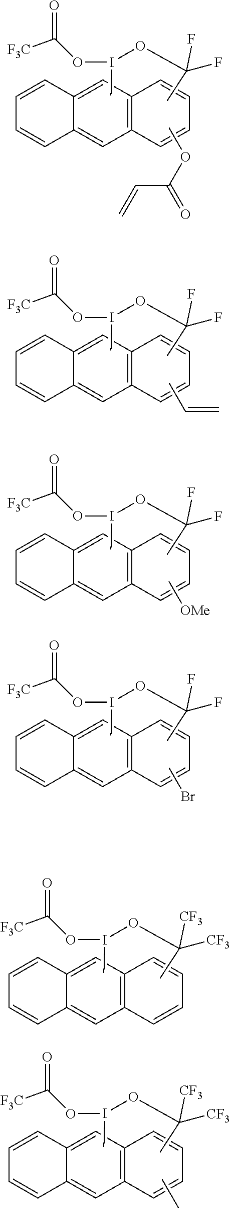

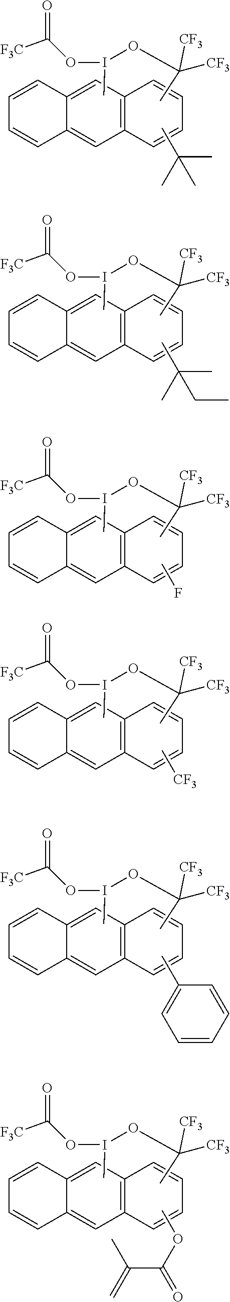

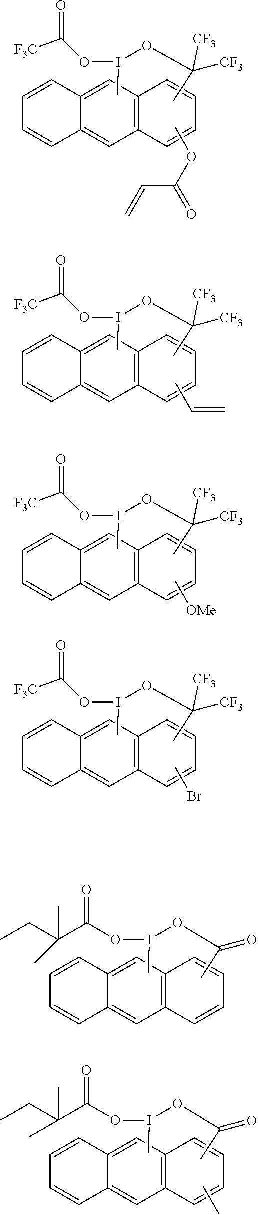

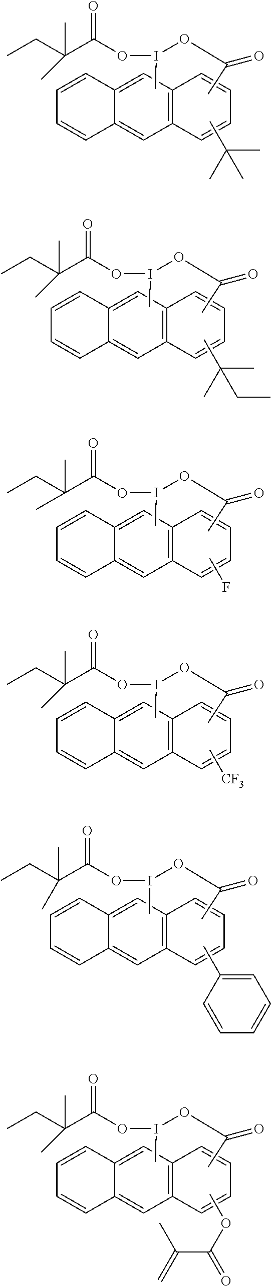

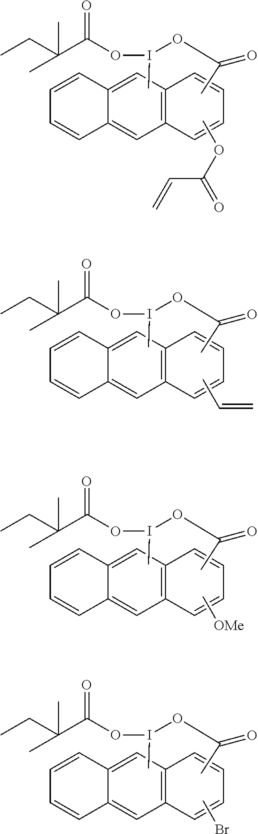

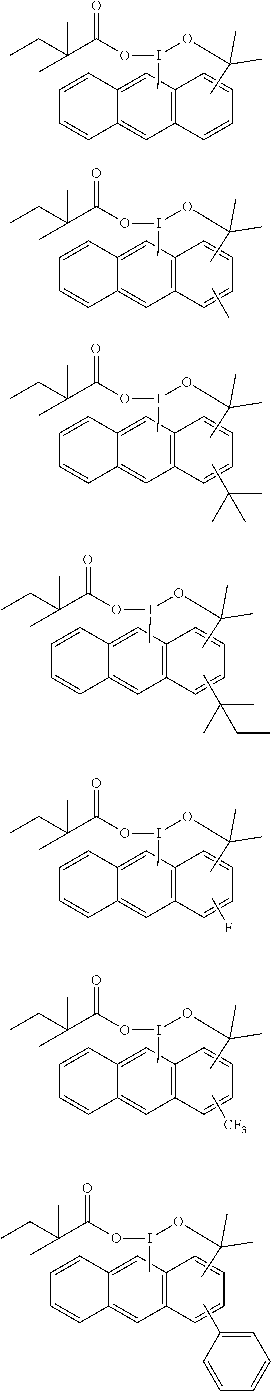

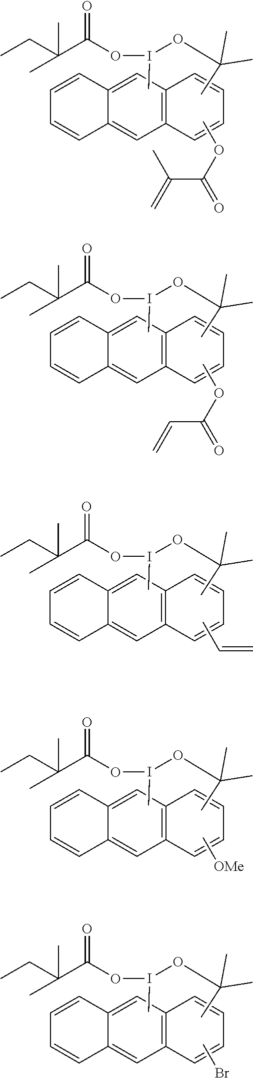

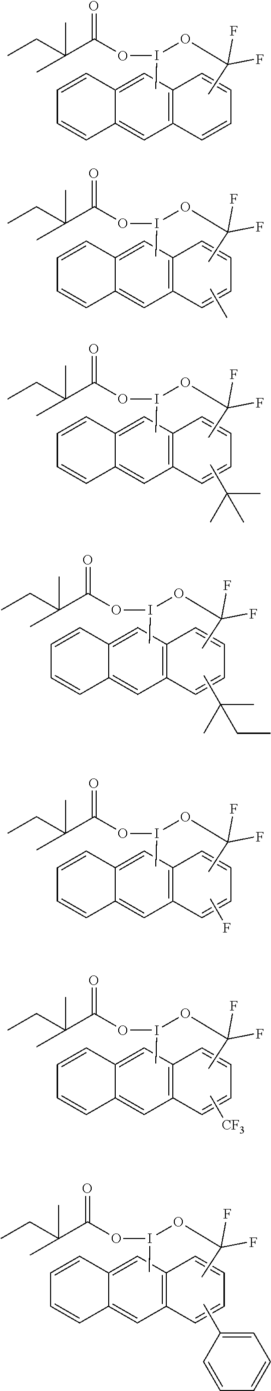

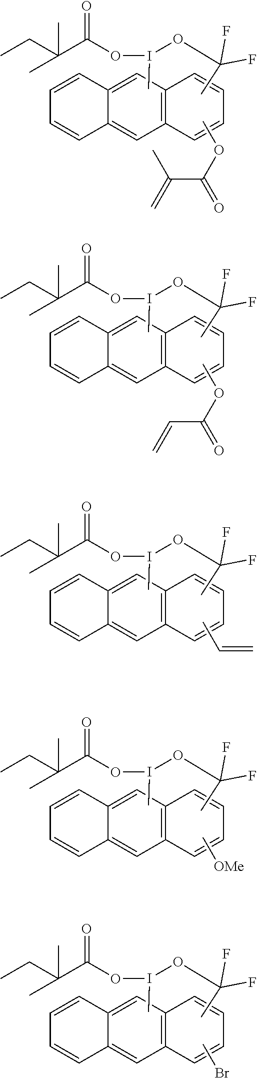

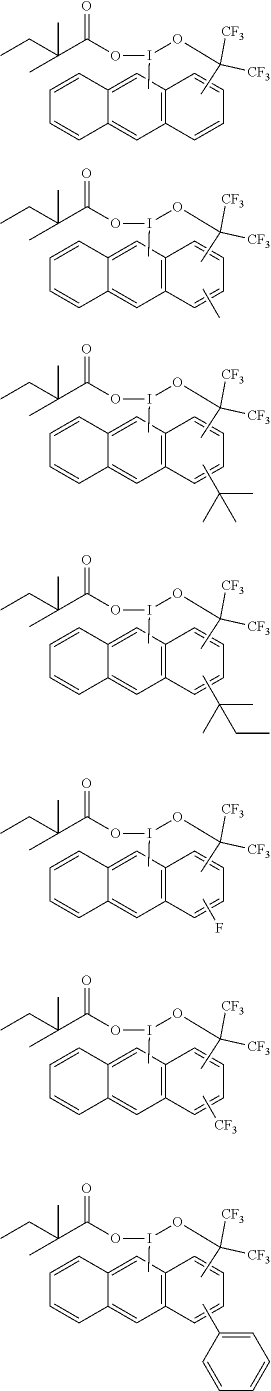

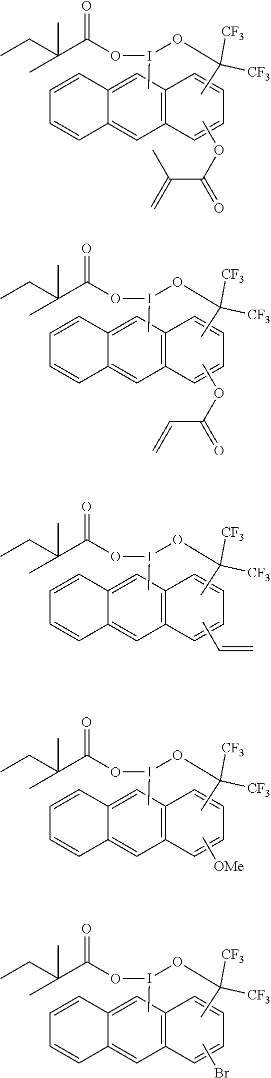

In the general formula (5), “m2” represents an integer of 0 to 2.

When “m2” is 0, “n2” represents an integer of 1 to 3, “n3” represents an integer of 0 to 5, and 1≤(n2+n3)≤6 is satisfied.

When “m2” is 1, “n2” represents an integer of 1 to 3, “n3” represents an integer of 0 to 7, and 1≤(n2+n3)≤8 is satisfied.

When “m2” is 2, “n2” represents an integer of 1 to 3, “n3” represents an integer of 0 to 9, and 1≤(n2+n3)≤10 is satisfied.

In the general formula (5), R⁶¹ and R⁶² each independently represent a halogen atom or a hydrocarbyl group having 1 to 10 carbon atoms and optionally containing a heteroatom, the R⁶¹ and the R⁶² optionally being bonded to each other to form a ring together with the carbon atoms bonded thereto and the atoms between the carbon atoms. Examples of the halogen atom include a fluorine atom, a chlorine atom, a bromine atom, and an iodine atom. The hydrocarbyl group having 1 to 10 carbon atoms may be saturated or unsaturated, and may be linear, branched, or cyclic. Specific examples thereof include: alkyl groups having 1 to 10 carbon atoms, such as a methyl group, an ethyl group, a propyl group, an isopropyl group, an n-butyl group, an isobutyl group, a sec-butyl group, a tert-butyl group, an n-pentyl group, a tert-pentyl group, an n-hexyl group, an n-octyl group, a 2-ethylhexyl group, an n-nonyl group, and an n-decyl group; cyclic saturated hydrocarbyl groups having 3 to 10 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.0^{2,6}]decanyl group, and an adamantyl group; alkenyl groups, such as a vinyl group and an allyl group; aryl groups having 6 to 10 carbon atoms, such as a phenyl group and a naphthyl group; and groups obtained by combining these groups. Furthermore, part or all of the hydrogen atoms of the hydrocarbyl group may be substituted with a group containing a heteroatom, such as an oxygen atom, a sulfur atom, a nitrogen atom, or a halogen atom, and part of the —CH₂— of the hydrocarbyl group may be substituted with a group containing a heteroatom such as an oxygen atom, a sulfur atom, or a nitrogen atom. The resulting hydrocarbyl group may contain a hydroxy group, a cyano group, a halogen atom, a carbonyl group, an ether bond, a thioether bond, an ester bond, a sulfonic acid ester bond, a carbonate bond, a carbamate bond, a lactone ring, a sultone ring, a carboxylic anhydride (—C(═O)—O—C(═O)—), etc. As the R⁶¹ and the R⁶², hydrocarbyl groups having 1 to 4 carbon atoms are preferable.

In the general formula (5), R⁶³ represents a halogen atom or a hydrocarbyl group having 1 to 40 carbon atoms and optionally containing a heteroatom. Examples of the halogen atom include a fluorine atom, a chlorine atom, a bromine atom, and an iodine atom. The hydrocarbyl group having 1 to 40 carbon atoms may be saturated or unsaturated, and may be linear, branched, or cyclic. Specific examples thereof include: alkyl groups having 1 to 40 carbon atoms, such as a methyl group, an ethyl group, a propyl group, an isopropyl group, an n-butyl group, an isobutyl group, a sec-butyl group, a tert-butyl group, an n-pentyl group, a tert-pentyl group, an n-hexyl group, an n-octyl group, a 2-ethylhexyl group, an n-nonyl group, and an n-decyl group; cyclic saturated hydrocarbyl groups having 3 to 40 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.0^{2,6}]decanyl group, an adamantyl group, and an adamantylmethyl group; and aryl groups having 6 to 40 carbon atoms, such as a phenyl group, a naphthyl group, and an anthracenyl group. Furthermore, part or all of the hydrogen atoms of the hydrocarbyl group may be substituted with a group containing a heteroatom, such as an oxygen atom, a sulfur atom, a nitrogen atom, or a halogen atom, and part of the —CH₂— of the hydrocarbyl group may be substituted with a group containing a heteroatom such as an oxygen atom, a sulfur atom, or a nitrogen atom. The resulting hydrocarbyl group may contain a hydroxy group, a cyano group, a halogen atom, a carbonyl group, an ether bond, a thioether bond, an ester bond, a sulfonic acid ester bond, a carbonate bond, a carbamate bond, a lactone ring, a sultone ring, a carboxylic anhydride (—C(═O)—O—C(═O)—), etc. When “n3” is 2 to 9, the R⁶³s are identical to or different from each other and the R⁶³s are optionally bonded to each other to form a ring together with the carbon atoms of the aromatic ring bonded to the R⁶³s.

Note that the R⁶³ can substitute any position in the aromatic rings in the above formulae.

Specific examples of the hypervalent iodine compound represented by the general formula (5) include the following, but are not limited thereto.

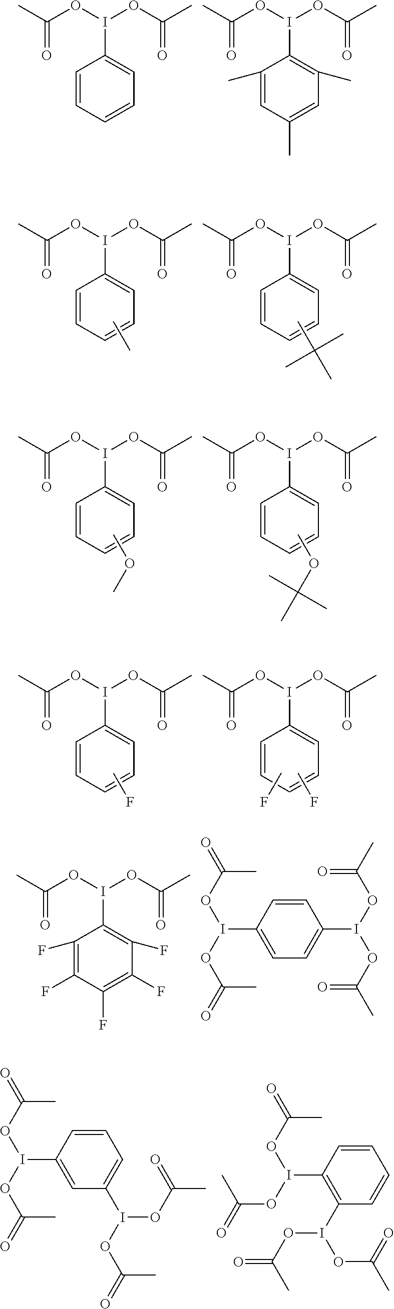

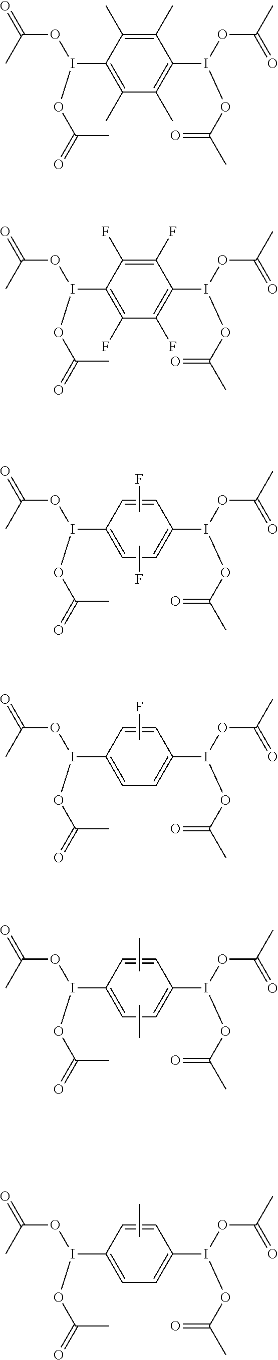

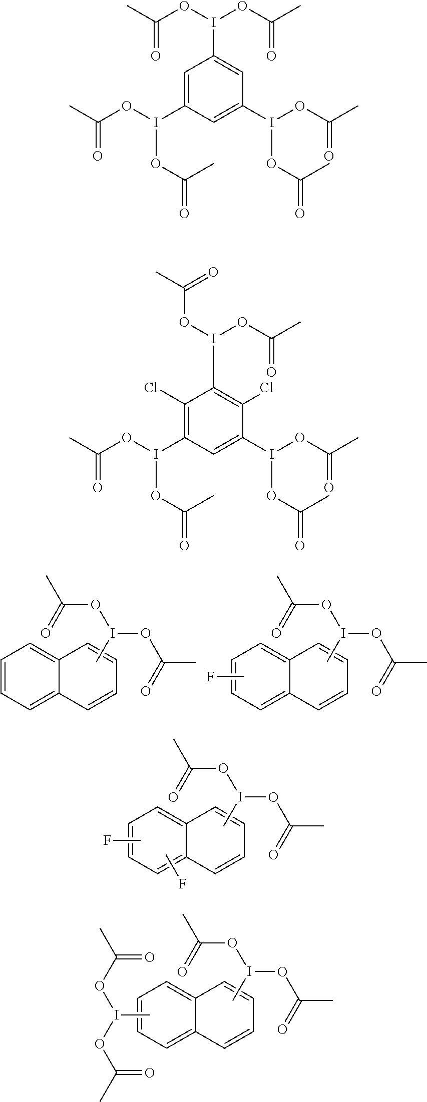

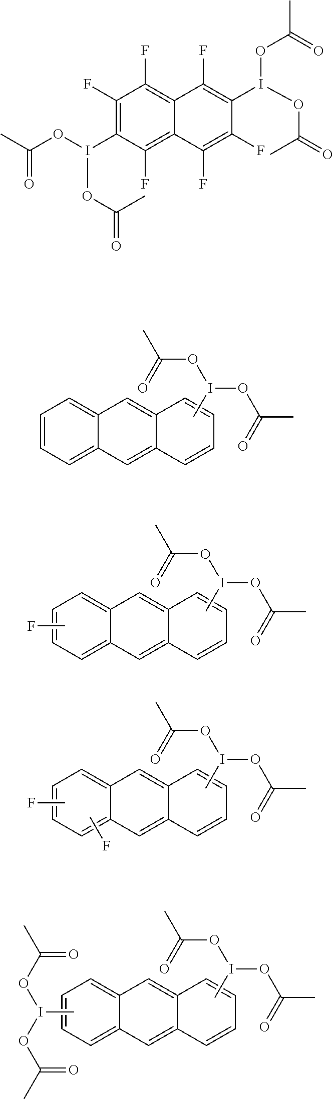

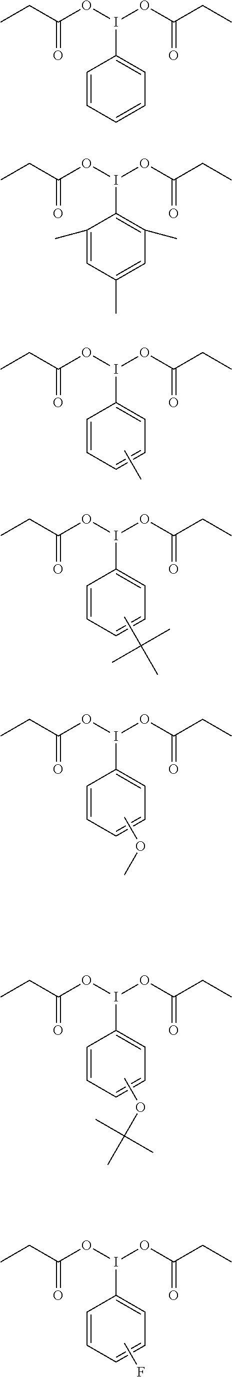

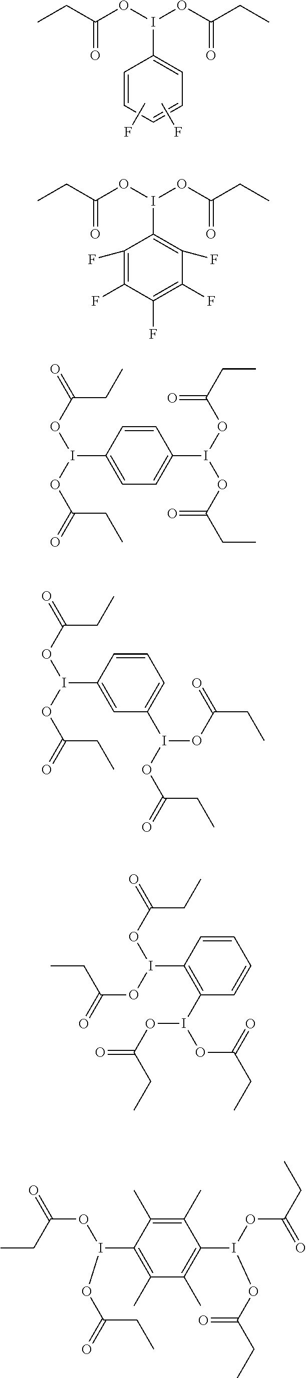

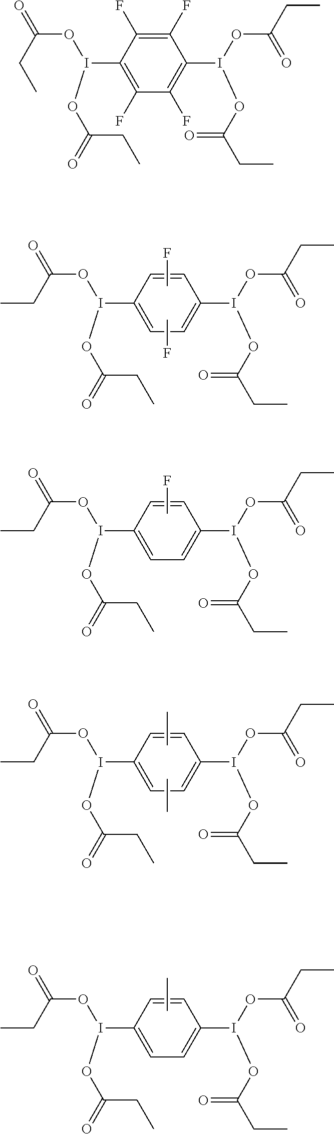

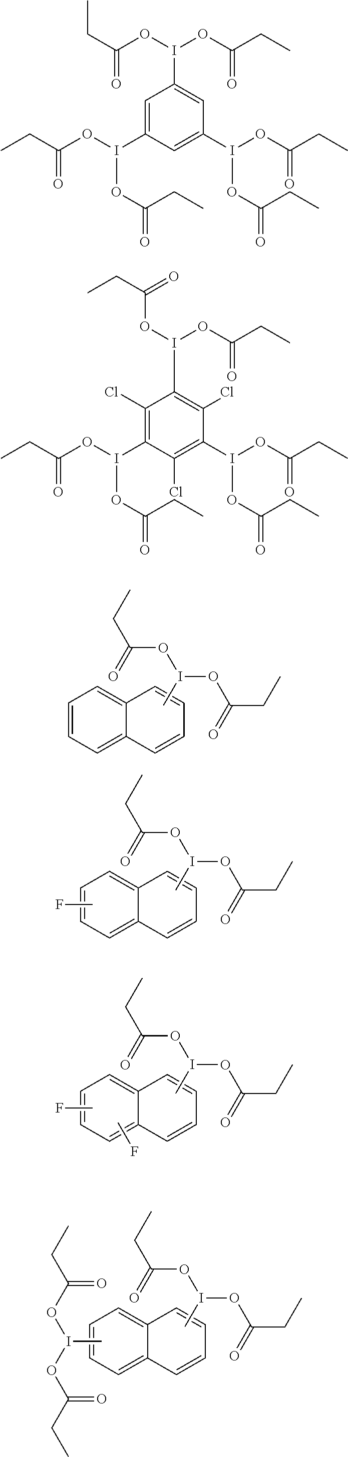

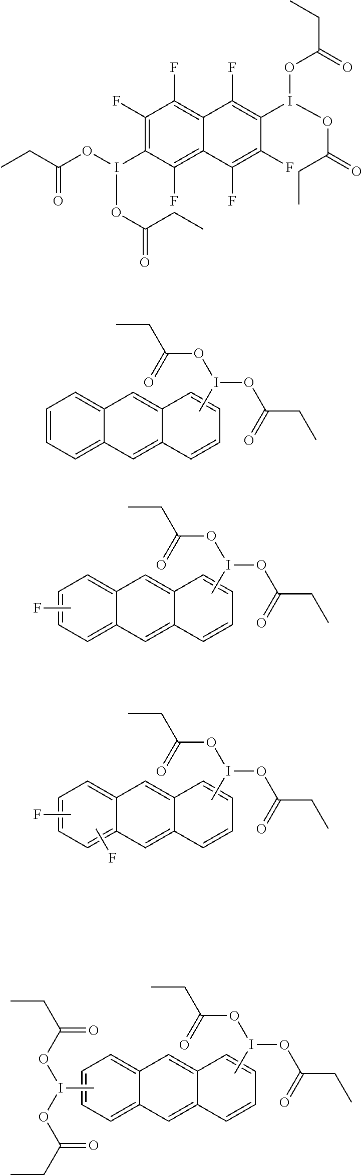

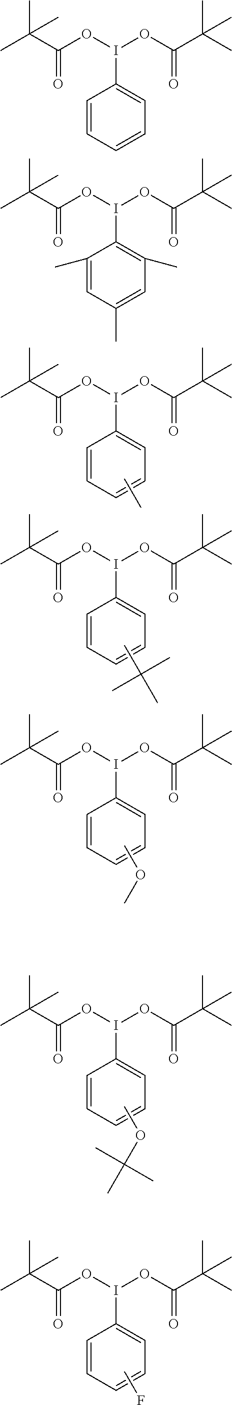

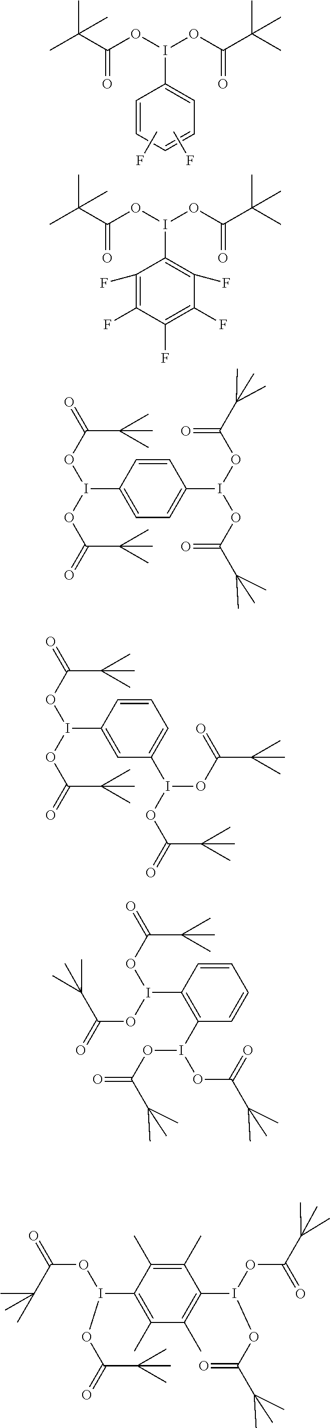

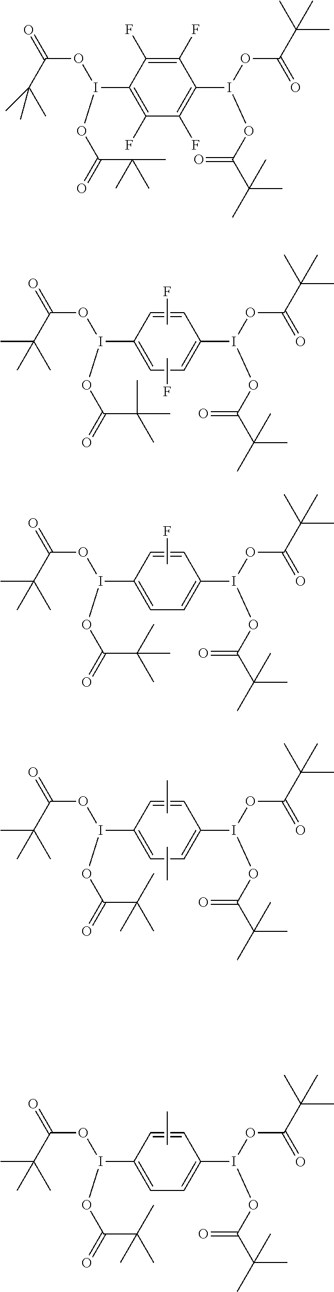

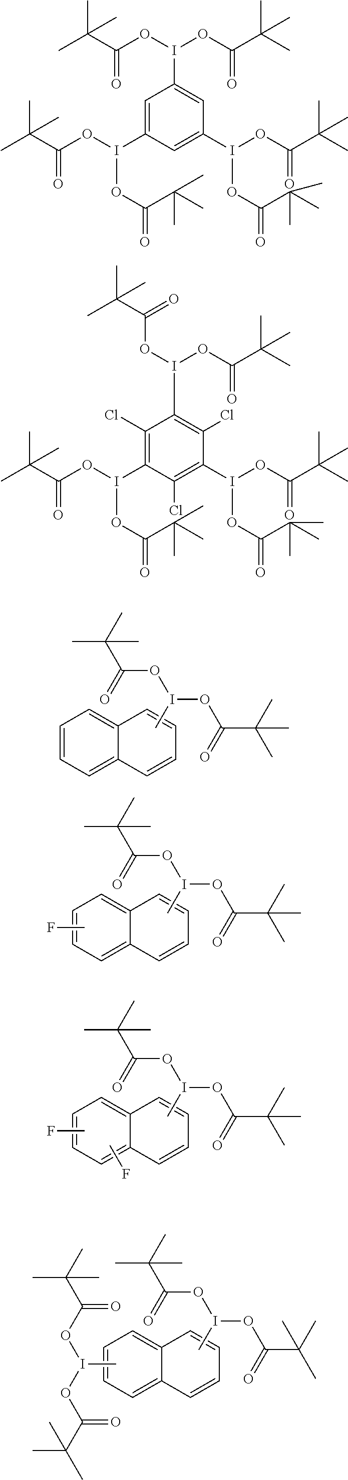

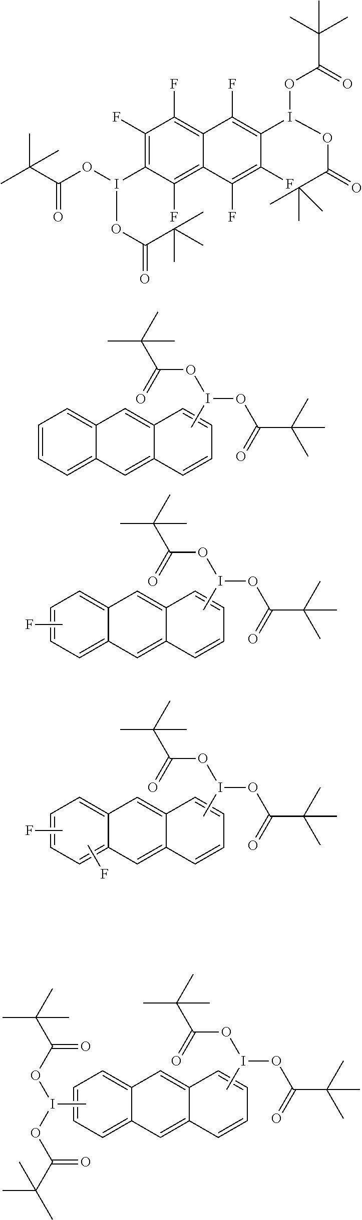

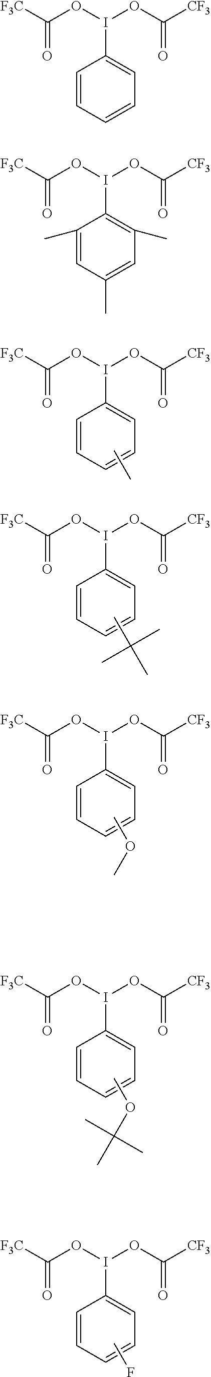

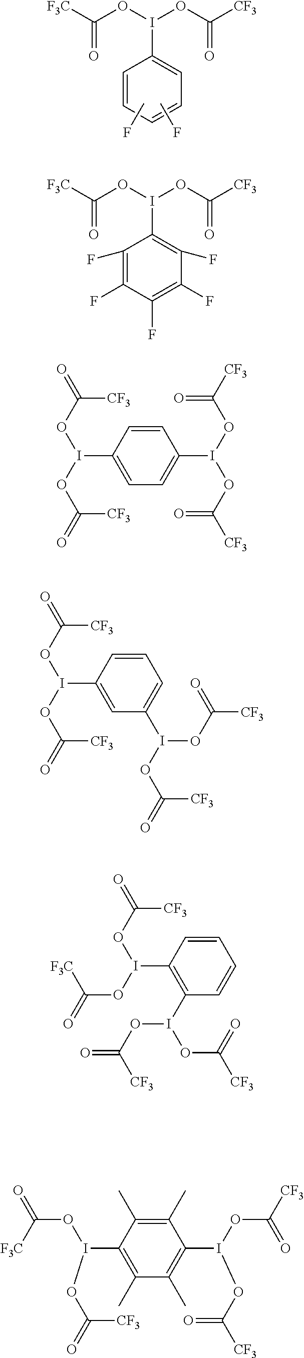

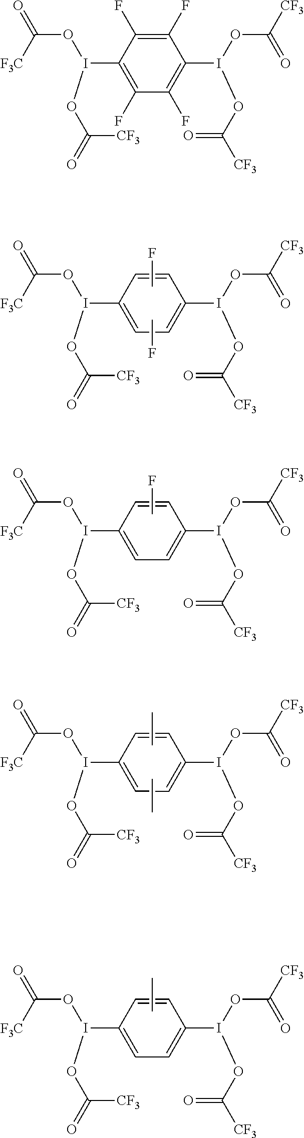

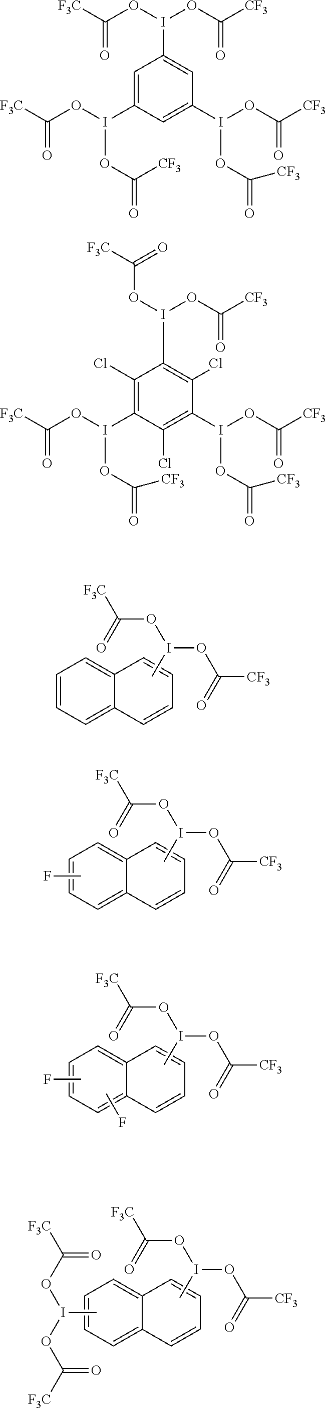

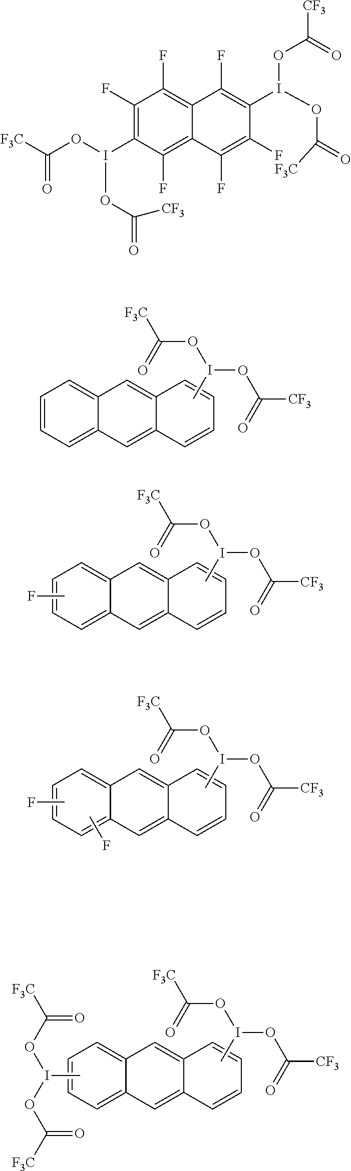

When the inventive resist composition contains other hypervalent iodine compounds, as the other hypervalent iodine compounds, the hypervalent iodine compound represented by the general formula (4) alone may be used, the hypervalent iodine compound represented by the general formula (5) alone may be used, or the hypervalent iodine compound represented by the general formula (4) and the hypervalent iodine compound represented by the general formula (5) may be used in combination. Furthermore, one kind of each of the hypervalent iodine compound represented by the general formula (4) and the hypervalent iodine compound represented by the general formula (5) may be used, or two or more different kinds thereof may be used in combination.

When the inventive resist composition contains other hypervalent iodine compounds, the content ratio of the hypervalent iodine compounds to the carboxy-group-containing compound (when the carboxy-group-containing compound is a carboxy-group-containing polymer, the content ratio of the hypervalent iodine compounds to the carboxylic-acid-containing repeating unit in the polymer) is preferably 1:99 to 99:1, more preferably 10:90 to 90:10, and further preferably 20:80 to 80:20 in molar ratio. Furthermore, the other hypervalent iodine compounds are preferably contained in such an amount that “other hypervalent iodine compounds”:“hypervalent iodine compound represented by the general formula (1)”=1:99 to 99:1, more preferably 1:99 to 50:50 relative to the hypervalent iodine compound represented by the general formula (1) in molar ratio.

### [Solvent]

The inventive resist composition contains a solvent. The solvent is not particularly limited as long as the solvent dissolves the hypervalent iodine compound represented by the general formula (1), the carboxy-group-containing compound, other hypervalent iodine compounds, and other components described later and allows film formation. As such a solvent, organic solvents are preferable, and specific examples thereof include: ketones, such as cyclohexanone, methyl-2-n-pentyl ketone, and methyl isoamyl ketone; alcohols, such as 3-methoxybutanol, 3-methyl-3-methoxybutanol, 1-methoxy-2-propanol, 1-ethoxy-2-propanol, diacetone alcohol, 4-methyl-2-pentanol, and methyl 2-hydroxyisobutyrate; ethers, such as propylene glycol monomethyl ether, ethylene glycol monomethyl ether, propylene glycol monoethyl ether, ethylene glycol monoethyl ether, propylene glycol dimethyl ether, and diethylene glycol dimethyl ether; esters, such as propylene glycol monomethyl ether acetate, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; carboxylic acids, such as formic acid, acetic acid, and propionic acid; lactones, such as γ-butyrolactone; and mixed solvents thereof.

In the inventive resist composition, the amount of the solvent contained is preferably such an amount that the concentration of the solid contents in the resist composition is 0.1 to 20 mass %, more preferably 0.1 to 15 mass %, and further preferably 0.1 to 10 mass %. Note that, in the present invention, solid contents is a general term for the components other than the solvent out of all the components of the resist composition.

One kind of the solvent may be used, or two or more kinds thereof may be used in mixture.

### [Other Components]

The inventive resist composition may further contain a surfactant. As the surfactant, a fluorine-based and/or silicone-based surfactant is preferable. Specific examples of such a surfactant include

surfactants disclosed in paragraph of US2008/0248425A1. Furthermore, it is also possible to use a surfactant disclosed in paragraph of US2008/0248425A1, other than the fluorine-based and/or silicone-based surfactants.

When the inventive resist composition contains the surfactant, the contained amount is preferably 0.0001 to 2 mass % of all the solid contents. One kind of the surfactant may be used, or two or more kinds thereof may be used in combination.

The inventive resist composition may further contain at least one selected from a radical scavenger and a crosslinking agent. In this manner, the photoreaction during photolithography can be controlled, and sensitivity can be adjusted.

Specific examples of the radical scavenger include hindered phenols, quinones, hindered amines, and thiol compounds. Specifically, specific examples of the hindered phenols include dibutylhydroxytoluene (BHT) and 2,2′-methylenebis(4-methyl-6-tert-butylphenol). Specific examples of the quinones include 4-methoxyphenol (methoquinone) and hydroquinone. Specific examples of the hindered amines include 2,2,6,6-tetramethylpiperidine and 2,2,6,6-tetramethylpiperidine-N-oxy radical. Specific examples of the thiols include dodecanethiol and hexadecanethiol.

When the inventive resist composition contains the radical scavenger, the contained amount is preferably 0.01 to 10 mass % of all the solid contents. One kind of the radical scavenger may be used, or two or more kinds thereof may be used in combination.

Specific examples of the crosslinking agent include compounds having a carbon-carbon unsaturated bond as a functional group, such as a vinyl group, a (meth)acrylate group, an allyl group, an alkynyl group, and an aromatic ring. Specifically, specific examples of compounds having a vinyl group include chain alkenes, branched alkenes, and cyclic alkenes, each optionally having a substituent. Specific examples of compounds having a (meth)acrylate group include acrylic acid, methacrylic acid, acrylic acid ester, and methacrylic acid ester, each optionally having a substituent. Specific examples of compounds having an allyl group include allyl alcohol, allyl ether, allyl ester, allyl amide, allylamine, and allyl-group-containing isocyanurates, each optionally having a substituent. Specific examples of compounds having an alkynyl group include chain alkynes, branched alkynes, cyclic alkynes, alkynyl alcohols, alkynyl ethers, alkynyl esters, alkynyl amides, alkynyl amines, and alkynyl-group-containing isocyanurates, each optionally having a substituent. Specific examples of compounds having an aromatic ring include arenes, heteroarenes, styrene, stilbene, phenylacetylene, acenaphthylene, and chalcone, each optionally having a substituent. The crosslinking agent may have only one of the functional groups, or may have a plurality of the groups. The number of the functional groups contained in the crosslinking agent is preferably 1 or more and 10 or less, more preferably 2 or more and 8 or less.

When the inventive resist composition contains the crosslinking agent, the contained amount is preferably 0.01 to 50 mass % of all the solid contents. One kind of the crosslinking agent may be used, or two or more kinds thereof may be used in combination.

The inventive resist composition mainly contains a hypervalent iodine compound and a carboxy-group-containing compound as described above, and base polymers containing acid-labile groups and photo-acid generators, which are contained in conventional chemically amplified resist compositions, do not need to be contained. However, in the inventive resist composition, a difference occurs in solubility between exposed portions and unexposed portions, and a positive pattern or a negative pattern can be formed, especially by exposure to EB or EUV. The mechanism is not completely clear, but the following conjecture can be made, for example.

The hypervalent iodine compound represented by the general formula (1) is a compound having tricoordinate hypervalent iodine having a carboxylate ligand. It can be assumed that when such a tricoordinate iodine compound is mixed with a carboxylic acid compound, exchange with the carboxylate ligand occurs by an equilibrium reaction. In this event, if the original carboxylate ligand can be removed by some method, a hypervalent iodine compound having a new ligand is generated. For example, when Dess-Martin periodinane, which is a hypervalent iodine compound that can be relatively easily acquired, and a carboxylic acid compound having a high molecular weight are mixed together and the generated acetic acid, having a low boiling point, is removed, ligand exchange is completed. Here, a polymer in which the carboxy-group-containing compound is crosslinked with the hypervalent iodine compound is obtained.

The polymer crosslinked with the hypervalent iodine compound is generated at the time of film formation. This is because such a crosslinked polymer is insoluble in many organic solvents, and therefore, a solution cannot be prepared even if the polymer is synthesized beforehand. This is conjectured to be because the hypervalent iodine compound, originally having low solvent solubility due to high polarization, contains the carboxy-group-containing compound as a ligand, and thus, solubility is even more degraded. Accordingly, in this step, it is desirable to remove the original low-molecular-weight carboxylic acid component at the time of film formation and in the subsequent baking process, thus completing the ligand exchange reaction and also forming a resist film.

The resist film of the present invention thus formed on a substrate changes in polarity by the hypervalent iodine compound, being the main component of the resist film, being decomposed by light, and a pattern is formed by a development process. Incidentally, by selecting the developer appropriately, a positive or negative pattern can be formed.

The inventive resist composition can be either a positive type or a negative type depending on the choice of components. In the case of a positive type, a polymer in which a hypervalent iodine compound is bonded is contained at the time of film formation. By the polymer being decomposed due to light, a monovalent iodine compound is formed, and at the same time, the bond between the carboxy-group-containing compound and the hypervalent iodine compound is removed, and the molecular weight is reduced. It is conjectured that, as a result, a positive pattern, where exposed portions are removed with an organic solvent, is formed.

On the other hand, in the case of a negative type, a polymer generated at the time of film formation, crosslinked with a hypervalent iodine compound, is contained. By the polymer being decomposed due to light, exchange of cross links or bonds occurs, and increase in molecular weight and polarity conversion occur. It is conjectured that, as a result, a negative pattern, where unexposed portions are removed with an aqueous alkaline solution, is formed.

From the above-described conjecture, it can be said that the inventive resist composition is a non-chemically amplified resist composition. The inventive resist composition does not require an acid-labile group-containing base polymer or a photo-acid generator, unlike conventional chemically amplified resist compositions. Therefore, adverse effects (e. g. image blurs) due to acid diffusion do not occur, and resolution of a fine pattern is possible.

The inventive resist composition is extremely effective, especially in EUV lithography. This is because the inventive resist composition has iodine atoms, which are capable of greatly absorbing EUV light, and the hypervalent iodine compound represented by the general formula (1) has, on an iodine atom, a carboxylate ligand with which the above-described ligand exchange may occur, and therefore, the crosslinking with the carboxy-group-containing compound after the film formation progresses with higher density, and there is a characteristic that the difference between unexposed portions and exposed portions in dissolution rate, that is, dissolution contrast, is greater than in a case where only other hypervalent iodine compounds are used. That is, the inventive resist composition can achieve high sensitivity, high resolution, and low LWR by virtue of these characteristics. Thus, when a non-chemically amplified resist containing a hypervalent iodine compound having an SF₅ group and a carboxylic acid compound (carboxy-group-containing compound) is used, the solubility of the entire resist is enhanced. Thus, patterning excellent in sensitivity and roughness resolution is possible.

As a resist composition for EUV lithography with which a fine pattern can be formed, a metal resist that mainly contains a compound of tin, which is a metal having a high absorbance of EUV light in the same manner as iodine atoms (e. g. Patent Document 2), is reported. However, as described above, such a metal resist has many issues such as insufficient solubility in a solvent, storage stability, and defects due to residues after etching caused by a metal element being contained. On the other hand, the inventive resist composition has an advantage over metal resists regarding defects, since a metal element is not used, and there are no problems regarding solubility in a solvent either. Moreover, the inventive resist composition is applicable in the case of either a positive type or a negative type, and therefore, has a wide range of uses. For example, in a contact hole formation process, a reversal process step is necessary after forming a pillar pattern in the case of a metal resist performed with negative development, but such a step is unnecessary in the case of a positive resist. Therefore, it can be said that the inventive resist composition is more useful than metal resists from the viewpoint of the simplicity and convenience of the process as well.

JP2015-180928A and JP2018-95853A disclose a resist composition containing a hypervalent iodine compound as an additive and a resist composition in which a hypervalent iodine compound is incorporated in the polymer skeleton of a base polymer. However, the only characteristic of the resist compositions disclosed in these Patent Documents is improvement of line edge roughness, and there is no mention whatsoever regarding the possibility that the hypervalent iodine compound may be photolysed, or the possibility that the hypervalent iodine compound may function as a material for a non-chemically amplified resist composition. Furthermore, according to the description regarding the contained amount and specific examples, the hypervalent iodine compound is not the main component. Meanwhile, Patent Document 3 proposes a positive resist composition containing a hypervalent iodine compound, but there is no description regarding the inventive hypervalent iodine compound represented by the general formula (1), and there is no mention whatsoever that resolution and LWR can be improved by using such a compound. Accordingly, it is considered that a non-chemically amplified resist composition that is extremely highly sensitive, exhibits excellent resolution, and is extremely effective for precise fine processing like the inventive composition cannot be conceived from these Patent Documents. That is, it can be said that the present invention provides a clearly novel resist composition and a clearly novel patterning process.

### [Laminate]

The present invention provides a laminate including: a substrate; and a resist film, which is a film body formed of the above-described resist composition, on the substrate. In such a laminate, including a resist film obtained from the non-chemically amplified resist composition of the present invention, the resist film, which is a film body of the above-described resist composition, has extremely high sensitivity, also exhibits excellent limiting resolution, is extremely effective for precise fine processing, and in addition, is applicable to either positive or negative patterning. Therefore, the laminate has a wide range of uses, and is extremely highly useful in resist process technology.

In this case, a resist underlayer film can be further provided as necessary between the substrate and the resist film.

Furthermore, in the inventive laminate, the resist film preferably contains a product made by a ligand exchange reaction of the hypervalent iodine compound and the carboxy-group-containing compound. That is, the laminate which is formed by a substrate and a resist film obtained from the inventive resist composition on the substrate is obtained and the resist film is preferably one formed by ligand exchange between the hypervalent iodine compound and the carboxy-group-containing compound.

As described above, by removing by-product low-molecular-weight carboxylic acid produced during film formation and in the subsequent baking process, the ligand exchange reaction between the hypervalent iodine compound and the carboxy-group-containing compound progresses, and a resist film containing a ligand exchange reaction product is formed (that is, a film body is produced). By the ligand exchange being completed, a polymer in which the carboxy-group-containing compound is crosslinked with the hypervalent iodine compound is obtained. It is preferable to form the resist film on completing the ligand exchange reaction in this manner.

### [Patterning Process]

When the inventive resist composition is used for manufacturing various integrated circuits, a known lithography technique can be applied. As the inventive patterning process, it is possible to provide a patterning process including the steps of: forming a resist film by using the above-described resist composition on a substrate or on a resist underlayer film of a substrate on which the resist underlayer film has been laminated; exposing the resist film by using a high-energy beam; and developing the exposed resist film by using a developer. Hereinafter, the resist underlayer film is also simply referred to as an “underlayer film”.

Firstly, the inventive resist composition is applied onto a substrate for manufacturing an integrated circuit, on an underlayer film of a substrate (Si, SiO₂, SiN, SiON, TiN, WSi, BPSG, SOG, organic antireflective film, etc.) on which the underlayer film has been laminated, on a substrate for manufacturing a mask circuit, or on an underlayer film of a substrate (Cr, Cro, CrON, MoSi₂, SiO₂, etc.) on which the underlayer film has been laminated, by an appropriate coating process, such as spin coating, roll coating, flow coating, dip coating, spray coating, or doctor coating, so that the thickness of the coating film is 0.01 to 2 μm. The resultant is prebaked on a hot plate preferably at 60 to 200° C. for 10 seconds to 30 minutes, more preferably 80 to 180° C. for 30 seconds to 20 minutes. Thus, a resist film is formed. Note that an underlayer film means a film formed between the substrate and the resist film in a multilayer resist process. The underlayer film is not particularly limited, and a conventionally known film can be used.

Subsequently, the resist film is exposed by using a high-energy beam. Examples of the high-energy beam include ultraviolet ray (g-line (436 nm), h-line (405 nm), i-line (365 nm), etc.), deep ultraviolet ray, EB, EUV, X-ray, soft X-ray, excimer laser beam (KrF excimer laser beam, ArF excimer laser beam, etc.), y-ray, and synchrotron radiation. In the inventive resist patterning process, as the high-energy beam, it is preferable to use an i-line, a KrF excimer laser beam, an ArF excimer laser beam, an electron beam, or an extreme ultraviolet ray. When ultraviolet ray, deep ultraviolet ray, EUV, X-ray, soft X-ray, excimer laser beam, γ-ray, synchrotron radiation, or the like is employed as the high-energy beam, the irradiation is performed directly or while using a mask for forming a target pattern at an exposure dose of preferably about 1 to 300 mJ/cm², more preferably about 10 to 200 mJ/cm². When an EB is employed as the high-energy beam, the writing is performed directly or while using a mask for forming a target pattern at an exposure dose of preferably about 0.1 to 2000 μC/cm², more preferably about 0.5 to 1500 μC/cm². Note that the inventive resist composition is particularly suitable for fine patterning with an EB or EUV, among the high-energy beams.

After the exposure, PEB is performed as necessary. In this event, the PEB is preferably performed after the exposure on a hot plate or in an oven under the conditions of 30 to 150° C. for 10 seconds to 30 minutes, more preferably 60 to 120° C. for 30 seconds to 20 minutes.

After the exposure or after the PEB, development is performed by using a developer as necessary to perform patterning. Examples of the developer used in this event include: aqueous alkaline solutions, such as an aqueous solution of tetramethylammonium hydroxide; and organic solvents, such as 2-octanone, 2-nonanone, 2-heptanone, 3-heptanone, 4-heptanone, 2-hexanone, 3-hexanone, diisobutyl ketone, methylcyclohexanone, acetophenone, methylacetophenone, isopropyl alcohol, isoamyl alcohol, n-butanol, n-pentanol, cyclohexanol, propyl acetate, butyl acetate, isobutyl acetate, pentyl acetate, butenyl acetate, isopentyl acetate, cyclohexyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, phenylmethyl acetate, phenylethyl acetate, benzyl formate, phenylethyl formate, methyl 3-phenylpropionate, benzyl propionate, 2-phenylethyl acetate, 1-propanol, 3-methoxybutanol, 3-methyl-3-methoxybutanol, 1-methoxy-2-propanol, 1-ethoxy-2-propanol, diacetone alcohol, and 4-methyl-2-pentanol. One kind of these developers may be used, or two or more kinds thereof may be used in mixture.

After the development, rinsing is performed as necessary. The rinsing liquid is preferably a solvent that is miscible with the developer but does not dissolve the resist film. As such a solvent, it is preferable to use an alcohol having 3 to 10 carbon atoms, an ether compound having 8 to 12 carbon atoms, and an alkane, alkene, alkyne, and aromatic solvent, each having 6 to 12 carbon atoms. Alternatively, water may be used instead of an organic solvent as a rinsing liquid.

The rinsing can reduce resist pattern collapse and defect formation. Meanwhile, the rinsing is not necessarily essential, and the amount of the solvent used can be reduced by not performing the rinsing.

In the inventive resist composition, a difference occurs in the solubility between exposed portions and unexposed portions by virtue of exposure as described above, and a positive or negative pattern can be formed. Therefore, it is possible to use a developer that dissolves exposed portions and does not dissolve unexposed portions, or a developer that dissolves unexposed portions and does not dissolve exposed portions. Thus, the inventive patterning process makes it possible to form a positive or negative pattern by appropriately selecting a developer, and therefore, is widely applicable to various kinds of fine patterning.

### EXAMPLES

Hereinafter, the present invention will be specifically described with reference to Examples and Comparative Examples, but the present invention is not limited to the Examples.

### [1] Preparation of Hypervalent Iodine Compounds

The hypervalent iodine compounds used in the Examples are represented by the following formulae I-1 and I-2.

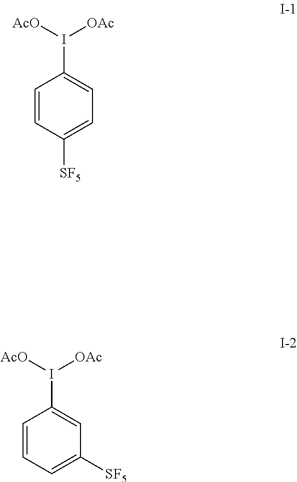

The above I-1 and I-2 were synthesized as follows.

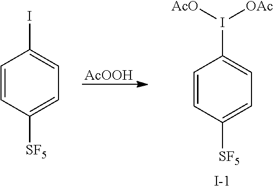

### [Synthesis Example 1-1] Synthesis of I-1

To 20 mL of 2.0-M peracetic acid (acetic anhydride solution), 4-iodophenylsulfur pentafluoride (5 g, 15.1 mmol) was added, and the mixture was stirred at 40° C. for 12 hours. The temperature was restored to room temperature, 100 mL of isopropyl ether (IPE) was added, and a solid was collected by filtration. The obtained solid was dried at 40° C. for 1 hour to obtain I-1 (3.1 g, 46% yield). The nuclear magnetic resonance spectrum and mass spectrum of the obtained I-1 were as follows.

¹H NMR: (500 MHz, CDCl₃) δ 8.18 (d, 2H), 7.85 (d, 2H), 2.01 (s, 6H) ppm.

Single quadrupole mass spectrometry (ESI): POSITIVE M+H⁺ 347.9 (C₁₁H₁₁INO₄ equivalent)

### [Synthesis Example 1-2] Synthesis of I-2

I-2 was also synthesized in the same manner as I-1.

### [2] Synthesis of Polymers

The monomers used for the synthesis of polymers are as follows.

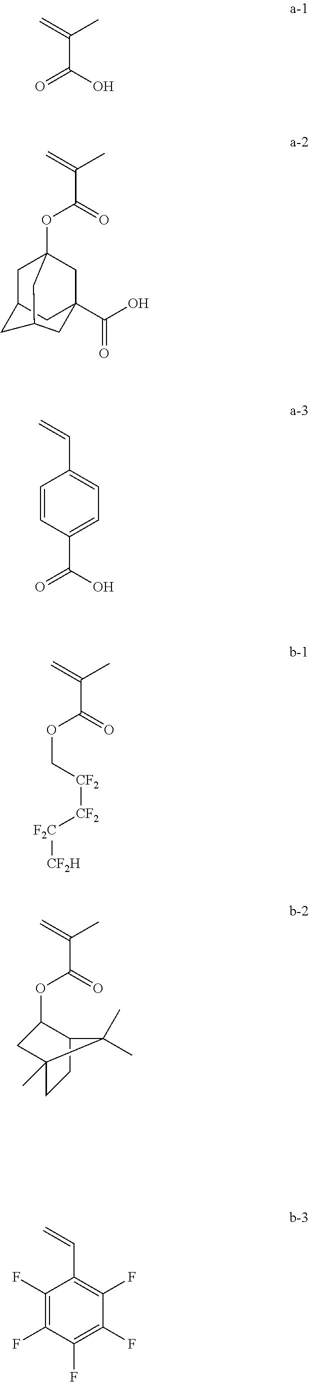

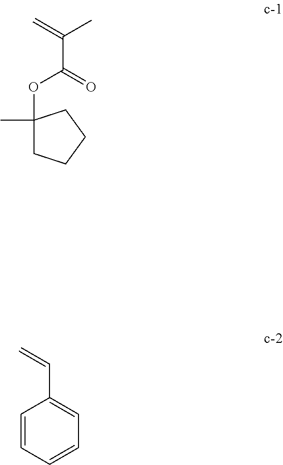

### [Synthesis Example 2-1] Synthesis of Polymer (P-1)

Under a nitrogen atmosphere, a monomer (a-1) (56 g), a monomer (b-1) (105 g), 5.4 g of V-601 (manufactured by FUJIFILM Wako Pure Chemical Corporation), and 180 g of MEK (methyl ethyl ketone) were added into a flask to prepare a monomer-polymerization initiator solution. Into another flask with a nitrogen atmosphere, 55 g of MEK was added and heated to 80° C. with stirring, and then the monomer-polymerization initiator solution was added dropwise over 4 hours. After the dropwise addition, the polymerization liquid was further stirred for 2 hours with maintaining the temperature at 80° C., and then cooled to room temperature. The obtained polymerization liquid was added dropwise to 4000 g of vigorously stirred hexane, and a precipitated polymer was collected by filtration. Furthermore, the obtained polymer was washed twice with 1200 g of hexane, and then dried in vacuo at 50° C. for 20 hours to obtain a white powder polymer (P-1) (155 g, 96% yield). The polymer (P-1) had Mw of 7700 and Mw/Mn of 1.82. The Mw and Mn are polystyrene-converted measurement values obtained by GPC using tetrahydrofuran (THE) as an eluent. Specifically, measurement was carried out under the following conditions (the same hereinafter). The results are shown in Table 1 below.
Apparatus: HLC-8320GPC
Column:
   +TSK guardcolumn
   +TSKgel G4000HXL
   +TSKgel G2000HXL
   +TSKgel superH5000
Constant temperature of pump and column: 40° C.
Eluent: THE
Detector: RI (refractive index) detector
Injection volume: 100 μl

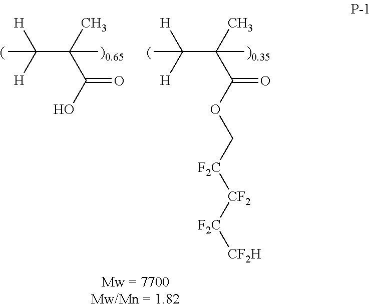

### [Synthesis Examples 2-2 to 2-10] Synthesis of Polymers (P-2 to P-10)

The polymers shown in Table 1 below were synthesized in the same manner as in Synthesis Example 2-1 except that the kinds and blending ratios of the monomers were changed. Note that the polymer (P-10) does not have a carboxy group (—COOH), and is not a carboxy-group-containing compound of the present invention.

TABLE 1

| | | | Introduction | | Introduction | | |
|---|---|---|---|---|---|---|---|
| | | | rate | | rate | | |
| | Polymer | Unit 1 | (mol % ) | Unit 2 | (mol %) | Mw | Mw/Mn |
| Synthesis | P-1 | a-1 | 65 | b-1 | 35 | 7700 | 1.82 |
| Example 2-1 | | | | | | | |
| Synthesis | P-2 | a-1 | 50 | b-2 | 50 | 8400 | 1.84 |
| Example 2-2 | | | | | | | |
| Synthesis | P-3 | a-1 | 60 | b-3 | 40 | 8100 | 1.79 |
| Example 2-3 | | | | | | | |
| Synthesis | P-4 | a-2 | 65 | b-1 | 35 | 8300 | 1.83 |
| Example 2-4 | | | | | | | |
| Synthesis | P-5 | a-2 | 50 | b-2 | 50 | 8300 | 1.83 |
| Example 2-5 | | | | | | | |
| Synthesis | P-6 | a-2 | 60 | b-3 | 40 | 8200 | 1.82 |
| Example 2-6 | | | | | | | |
| Synthesis | P-7 | a-3 | 65 | b-1 | 35 | 8000 | 1.80 |
| Example 2-7 | | | | | | | |
| Synthesis | P-8 | a-3 | 50 | b-2 | 50 | 8600 | 1.84 |
| Example 2-8 | | | | | | | |
| Synthesis | P-9 | a-3 | 60 | b-3 | 40 | 7900 | 1.81 |
| Example 2-9 | | | | | | | |
| Synthesis | P-10 | c-1 | 60 | c-2 | 40 | 9800 | 1.82 |
| Example 2-10 | | | | | | | |

### [3] Preparation of Resist Compositions

### Examples 1-1 to 1-22 and Comparative Examples 1-1 to 1-4

A hypervalent iodine compound, another hypervalent iodine compound, and a carboxy-group-containing compound were dissolved in a solvent containing 0.01 mass % of a surfactant (PF-636, manufactured by Omnova Solutions Inc.) in the constitution shown below in Table 2, and the obtained solution was filtered with a 0.2-μm Teflon (registered trademark) filter to prepare resist compositions (R-01 to R-22) and comparative resist compositions (CR-01 and CR-02). Meanwhile, a polymer, a photo-acid generator, and a sensitivity modifier were dissolved in a solvent containing 0.01 mass % of a surfactant (PF-636, manufactured by Omnova Solutions Inc.) in the constitution shown below in Table 3, and the obtained solution was filtered with a 0.2-μm Teflon (registered trademark) filter to prepare comparative resist compositions (CR-03 and CR-04).

TABLE 2

| | | | Other | Carboxy- | | |
|---|---|---|---|---|---|---|
| | | Hypervalent | hypervalent | group- | | |
| | | iodine | iodine | containing | | |
| | | compound | compound | compound | Solvent 1 | Solvent 2 |
| | Resist | (parts by | (parts by | (parts by | (parts by | (parts by |
| | composition | mass) | mass ) | mass) | mass) | mass ) |
| Example 1-1 | R-01 | I-1 (10) | — | P-1 (17.4) | PGMEA (800) | AcOH (200) |
| Example 1-2 | R-02 | I-2 (10) | — | P-1 (13.2) | PGMEA (800) | AcOH (200) |
| Example 1-3 | R-03 | I-1 (10) | — | P-2 (21.9) | PGMEA (800) | AcOH (200) |
| Example 1-4 | R-04 | I-1 (10) | — | P-3 (15.2) | PGMEA (800) | AcOH (200) |
| Example 1-5 | R-05 | I-1 (10) | — | P-4 (24.3) | PGMEA (800) | AcOH (200) |
| Example 1-6 | R-06 | I-1 (10) | — | P-5 (34.5) | PGMEA (800) | AcOH (200) |
| Example 1-7 | R-07 | I-1 (10) | — | P-6 (27.9) | PGMEA (800) | AcOH (200) |
| Example 1-8 | R-08 | I-1 (10) | — | P-7 (21.6) | PGMEA (800) | AcOH (200) |
| Example 1-9 | R-09 | I-1 (10) | — | P-8 (26.3) | PGMEA (800) | AcOH (200) |
| Example 1-10 | R-10 | I-1 (10) | — | P-9 (19.6) | PGMEA (800) | AcOH (200) |
| Example 1-11 | R-11 | I-1 (10) | — | P-1 (17.5) | HBM (800) | AcOH (200) |
| Example 1-12 | R-12 | I-1 (10) | — | P-1 (17.7) | PGMEA (800) | PA (200) |
| Example 1-13 | R-13 | I-1 (5) | — | P-1 (17.7) | PGMEA (800) | AcOH (200) |
| Example 1-14 | R-14 | I-1 (5) | 0-1 (2.5) | P-1 (17.5) | PGMEA (800) | AcOH (200) |
| Example 1-15 | R-15 | I-1 (5) | — | P-1 (17.7) | PGMEA (800) | AcOH (200) |
| | | I-2 (5) | | | | |
| Example 1-16 | R-16 | I-1 (10) | — | m-1 (8.6) | PGMEA (800) | ACOH (200) |
| Example 1-17 | R-17 | I-1 (10) | — | m-2 (4.1) | PGMEA (800) | AcOH (200) |
| Example 1-18 | R-18 | I-1 (10) | — | m-3 (8.4) | PGMEA (800) | AcOH (200) |
| Example 1-19 | R-19 | I-1 (10) | — | m-4 (7.6) | PGMEA (800) | AcOH (200) |
| Example 1-20 | R-20 | I-1 (10) | — | m-5 (11.5) | PGMEA (800) | AcOH (200) |
| Example 1-21 | R-21 | I-1 (10) | — | m-6 (8.8) | PGMEA (800) | AcOH (200) |
| Example 1-22 | R-22 | I-2 (10) | — | m-6 (6.9) | PGMEA (800) | AcOH (200) |
| Comparative | CR-01 | — | 0-1 (10) | P-1 (15) | PGMEA (800) | AcOH (200) |
| Example 1-1 | | | | | | |
| Comparative | CR-02 | — | 0-1 (10) | m-1 (7.6) | PGMEA (800) | AcOH (200) |
| Example 1-2 | | | | | | |

TABLE 3

| | | | Photo-acid | Sensitivity | | |
|---|---|---|---|---|---|---|
| | | Polymer | generator | modifier | Solvent 1 | Solvent 2 |
| | Resist | (parts by | (parts by | (parts by | (parts by | (parts by |
| | composition | mass) | mass ) | mass ) | mass ) | mass ) |
| Comparative | CR-03 | P-10 (80) | PAG-1 (19) | Q-1 (6) | PGMEA (1890) | GBL (210) |
| Example 1-3 | | | | | | |
| Comparative | CR-04 | P-10 (80) | PAG-1 (19) | Q-1 (6) | PGMEA (1890) | GBL (210) |
| Example 1-4 | | | | | | |

In the above Tables 2 and 3, the other hypervalent iodine compound (O-1), carboxy-group-containing compounds (m-1 to m-6), photo-acid generator (PAG-1), sensitivity modifier (Q-1), and solvents are as follows.

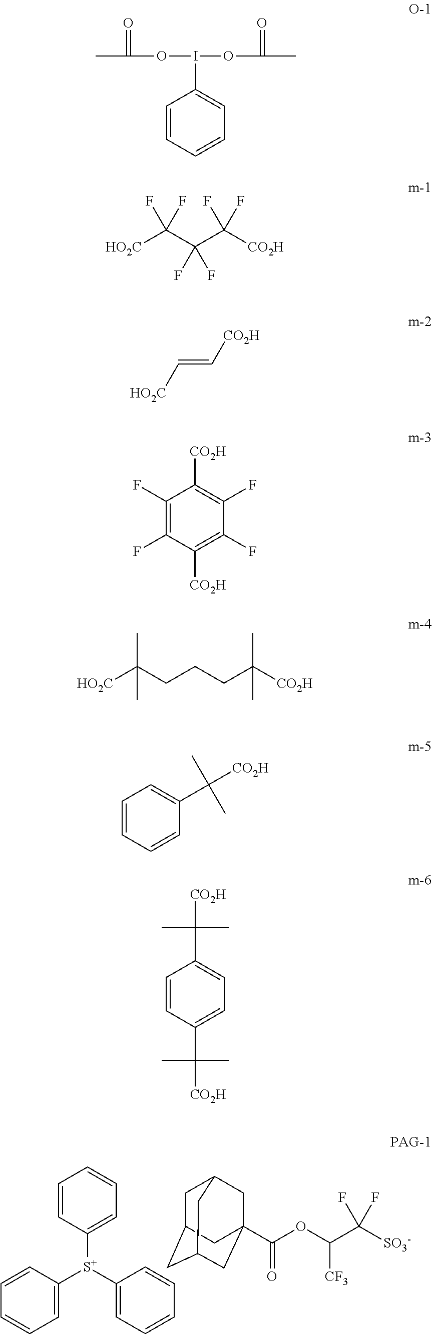

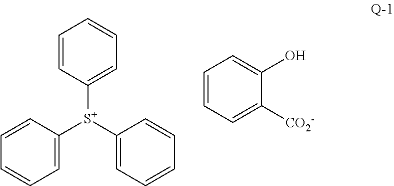

### Solvents:

PGMEA (propylene glycol monomethyl ether acetate)
AcOH (acetic acid)
HBM (methyl 2-hydroxyisobutyrate)
PA (propionic acid)
GBL (γ-butyrolactone)

### [4] EUV Lithography Evaluation (Line-and-Space Pattern, Positive Tone Development)

### Examples 2-1 to 2-22 and Comparative Examples 2-1 to 2-4

Each of the resist compositions (R-01 to R-22 and CR-01 to CR-04) was applied by spin-coating on a Si substrate on which a silicon-containing spin-on hard mask SHB-A940, manufactured by Shin-Etsu Chemical Co., Ltd. (silicon content of 43 mass %), was formed with 20 nm in film thickness, and subjected to post apply bake (PAB) by using a hot plate at the temperature shown in Table 4 below for 60 seconds to form a resist film having a film thickness of 40 nm. The resist film was exposed using an EUV scanner NXE3400 (NA 0.33, σ 0.9, 90° dipole illumination), manufactured by ASML Holding N.V., to form a 36-nm 1:1 line-and-space (LS) pattern. Then, PEB was performed on a hot plate at the temperature shown in Table 4 below for 60 seconds, and then development was performed with the developer shown in Table 4 for 30 seconds to form an LS pattern having a space width of 18 nm and a pitch of 36 nm.

Regarding the obtained resist pattern, the following evaluations were carried out. The results are shown in Table 4 below.

### [Sensitivity Evaluation]

The LS pattern was observed using a length-measurement SEM (CG-6300), manufactured by Hitachi High-Technologies Corporation, and an optimum exposure dose Eop (mJ/cm²) to yield the LS pattern with 18 nm in space width and 36 nm in pitch was determined to specify this value as sensitivity.

### [LWR Evaluation]

In the LS pattern obtained by irradiation at the optimum exposure dose, sizes in ten positions in the longitudinal direction of the space width were measured with a length-measurement SEM (CG-6300), manufactured by Hitachi High-Technologies Corporation. From the results, a tripled value (3σ) of a standard variation (σ) was determined as LWR. A smaller LWR value can yield a pattern with lower roughness and a more uniform space width.

### [Limiting Resolution Evaluation]

A pattern was formed while gradually increasing the exposure dose from the optimum exposure dose at which the LS pattern can be formed, and in this event, the limit of the line width (nm) at which resolution was possible was determined using a length-measurement SEM (CG-6300), manufactured by Hitachi High-Technologies Corporation, to specify this value as limiting resolution (nm). A smaller value indicates that it is possible to form a finer pattern with better limiting resolution.

TABLE 4

| | | | | | | Limiting |
|---|---|---|---|---|---|---|
| | Resist | PAB/PEB | | Eop | LWR | resolution |
| | composition | ( ° C.) | Developer | (mJ/cm²) | (nm) | (nm) |
| Example 2-1 | R-01 | 130/90 | nBA | 29 | 2.6 | 10 |
| Example 2-2 | R-02 | 130/90 | nBA | 27 | 3.0 | 10 |
| Example 2-3 | R-03 | 130/90 | nBA | 28 | 2.9 | 11 |
| Example 2-4 | R-04 | 130/90 | nBA | 32 | 2.7 | 9 |
| Example 2-5 | R-05 | 130/90 | nBA | 33 | 2.8 | 9 |
| Example 2-6 | R-06 | 130/90 | nBA | 34 | 2.7 | 12 |
| Example 2-7 | R-07 | 130/90 | nBA | 31 | 2.8 | 13 |
| Example 2-8 | R-08 | 130/90 | nBA | 33 | 2.6 | 12 |
| Example 2-9 | R-09 | 130/90 | nBA | 29 | 2.5 | 11 |
| Example 2-10 | R-10 | 130/90 | nBA | 31 | 2.7 | 11 |
| Example 2-11 | R-11 | 130/90 | nBA | 25 | 2.3 | 11 |
| Example 2-12 | R-12 | 130/90 | nBA | 25 | 2.2 | 8 |
| Example 2-13 | R-13 | 130/90 | CHA | 19 | 2.1 | 8 |
| Example 2-14 | R-14 | 130/90 | nBA | 22 | 2.2 | 7 |
| Example 2-15 | R-15 | 130/90 | nBA | 29 | 2.3 | 9 |
| Example 2-16 | R-16 | 130/90 | nBA | 29 | 2.7 | 9 |
| Example 2-17 | R-17 | 130/90 | nBA | 27 | 2.4 | 10 |
| Example 2-18 | R-18 | 130/90 | nBA | 28 | 2.9 | 11 |
| Example 2-19 | R-19 | 130/90 | nBA | 26 | 2.6 | 12 |
| Example 2-20 | R-20 | 130/90 | nBA | 26 | 2.5 | 9 |
| Example 2-21 | R-21 | 130/90 | nBA | 27 | 2.8 | 9 |
| Example 2-22 | R-22 | 130/90 | nBA | 29 | 2.2 | 11 |
| Comparative | CR-01 | 130/90 | nBA | 39 | 3.6 | 14 |
| Example 2-1 | | | | | | |
| Comparative | CR-02 | 130/90 | nBA | 40 | 3.8 | 16 |
| Example 2-2 | | | | | | |
| Comparative | CR-03 | 105/90 | TMAH | 80 | 4.5 | 18 |
| Example 2-3 | | | | | | |
| Comparative | CR-04 | 105/90 | TMAH | 85 | 4.8 | 18 |
| Example 2-4 | | | | | | |

### Developers:

nBA (butyl acetate)
CHA (cyclohexyl acetate)
TMAH (2.38 mass % aqueous solution of tetramethylammonium hydroxide)

### [5] EUV Lithography Evaluation (Line-and-Space Pattern, Negative Tone Development)

### Examples 3-1 to 3-22 and Comparative Examples 3-1 to 3-4

Each of the resist compositions (R-01 to R-22 and CR-01 to CR-04) was applied by spin-coating on a Si substrate on which a silicon-containing spin-on hard mask SHB-A940, manufactured by Shin-Etsu Chemical Co., Ltd. (silicon content of 43 mass %), was formed with 20 nm in film thickness, and subjected to post apply bake (PAB) by using a hot plate at the temperature shown in Table 5 below for 60 seconds to form a resist film having a film thickness of 40 nm. The resist film was exposed using an EUV scanner NXE3400 (NA 0.33, σ 0.9, 90° dipole illumination), manufactured by ASML Holding N.V., to form a 36-nm 1:1 line-and-space (LS) pattern. Then, PEB was performed on a hot plate at the temperature shown in Table 5 below for 60 seconds, and then development was performed with the developer shown in Table 5 below for 30 seconds to form an LS pattern having a space width of 18 nm and a pitch of 36 nm.

Regarding the obtained resist pattern, the following evaluations were carried out. The results are shown in Table 5 below.

### [Sensitivity Evaluation]

The LS pattern was observed using a length-measurement SEM (CG-6300), manufactured by Hitachi High-Technologies Corporation, and an optimum exposure dose Eop (mJ/cm²) to yield the LS pattern with 18 nm in space width and 36 nm in pitch was determined to specify this value as sensitivity.

### [LWR Evaluation]

In the LS pattern obtained by irradiation at the optimum exposure dose, sizes in ten positions in the longitudinal direction of the space width were measured with a length-measurement SEM (CG-6300), manufactured by Hitachi High-Technologies Corporation. From the results, a tripled value (30) of a standard variation (σ) was determined as LWR. A smaller LWR value can yield a pattern with lower roughness and a more uniform space width.

### [Limiting Resolution Evaluation]

A pattern was formed while gradually increasing the exposure dose from the optimum exposure dose at which the LS pattern can be formed, and in this event, the limit of the line width (nm) at which resolution was possible was determined using a length-measurement SEM (CG-6300), manufactured by Hitachi High-Technologies Corporation, to specify this value as limiting resolution (nm). A smaller value indicates that it is possible to form a finer pattern with better limiting resolution.

TABLE 5

| | | | | | | Limiting |
|---|---|---|---|---|---|---|
| | Resist | PAB/PEB | | Eop | LWR | resolution |
| | composition | ( ° C.) | Developer | (mJ/cm²) | (nm) | (nm) |
| Example 3-1 | R-01 | 130/90 | TMAH | 30 | 3.1 | 12 |
| Example 3-2 | R-02 | 130/90 | TMAH | 31 | 3.5 | 12 |
| Example 3-3 | R-03 | 130/90 | TMAH | 32 | 3.4 | 9 |
| Example 3-4 | R-04 | 130/90 | TMAH | 32 | 3.4 | 10 |
| Example 3-5 | R-05 | 130/90 | TMAH | 33 | 2.8 | 10 |
| Example 3-6 | R-06 | 130/90 | TMAH | 34 | 3.1 | 11 |
| Example 3-7 | R-07 | 130/90 | TMAH | 31 | 3.2 | 14 |
| Example 3-8 | R-08 | 130/90 | TMAH | 32 | 3.3 | 12 |
| Example 3-9 | R-09 | 130/90 | TMAH | 29 | 3.0 | 13 |
| Example 3-10 | R-10 | 130/90 | TMAH | 29 | 3.1 | 11 |
| Example 3-11 | R-11 | 130/90 | TMAH | 29 | 2.9 | 14 |
| Example 3-12 | R-12 | 130/90 | TMAH | 29 | 2.9 | 9 |
| Example 3-13 | R-13 | 130/90 | TMAH | 22 | 2.8 | 10 |
| Example 3-14 | R-14 | 130/90 | TMAH | 25 | 2.7 | 9 |
| Example 3-15 | R-15 | 130/90 | TMAH | 28 | 2.8 | 10 |
| Example 3-16 | R-16 | 130/90 | TMAH | 31 | 3.3 | 10 |
| Example 3-17 | R-17 | 130/90 | TMAH | 26 | 3.2 | 11 |
| Example 3-18 | R-18 | 130/90 | TMAH | 31 | 3.3 | 11 |
| Example 3-19 | R-19 | 130/90 | TMAH | 28 | 3.1 | 13 |
| Example 3-20 | R-20 | 130/90 | TMAH | 26 | 3.1 | 10 |
| Example 3-21 | R-21 | 130/90 | TMAH | 28 | 3.4 | 10 |
| Example 3-22 | R-22 | 130/90 | TMAH | 28 | 2.9 | 11 |
| Comparative | CR-01 | 130/90 | TMAH | 40 | 4.1 | 16 |
| Example 3-1 | | | | | | |
| Comparative | CR-02 | 130/90 | TMAH | 41 | 4.2 | 17 |
| Example 3-2 | | | | | | |
| Comparative | CR-03 | 105/90 | nBA | 83 | 4.6 | 18 |
| Example 3-3 | | | | | | |
| Comparative | CR-04 | 105/90 | nBA | 86 | 4.9 | 18 |
| Example 3-4 | | | | | | |

From the results shown in Tables 4 and 5 above, it was found that the inventive resist composition was excellent in sensitivity, LWR, and resolution in the formation of line-and-space patterns by EUV exposure in cases of either positive tone or negative tone development.

### [6] EUV Lithography Evaluation (Contact Hole Pattern)

### Examples 4-1 to 4-22 and Comparative Examples 4-1 to 4-4

Each of the resist compositions (R-01 to R-22 and CR-01 to CR-04) was applied by spin-coating on a Si substrate on which a silicon-containing spin-on hard mask SHB-A940, manufactured by Shin-Etsu Chemical Co., Ltd. (silicon content of 43 mass %), was formed with 20 nm in film thickness. Then, post apply bake (PAB) was performed at the temperature shown in Table 6 below for 60 seconds using a hot plate to produce a resist film with 50 nm in film thickness. Subsequently, the resist film was exposed using an EUV scanner NXE3400 (NA 0.33, σ 0.9/0.6, quadrupole illumination, 64 nm in pitch on wafer size, hole pattern mask with +20% bias), manufactured by ASML Holding N. V. Then, PEB was performed at the temperature shown in Table 6 below for 60 seconds on a hot plate. Thereafter, development was performed with the developer shown in Table 6 below for 30 seconds to obtain a hole pattern with 32 nm in size.

Regarding the obtained resist pattern, the following evaluations were carried out. The results are shown in Table 6 below.

### [Sensitivity Evaluation]

The contact hole pattern was observed using a length-measurement SEM (CG-6300), manufactured by Hitachi High-Technologies Corporation, and an optimum exposure dose Eop (mJ/cm²) to yield the hole pattern with a size of 32 nm was determined.

### [Critical Dimension Uniformity (CDU) Evaluation]

Sizes of fifty hole patterns obtained by irradiation at the optimum exposure dose were measured, and a tripled value (30) of a standard variation (o) calculated from the results was determined as CDU. A smaller CDU value can yield a pattern having a more uniform hole diameter.

### [Limiting Resolution Evaluation]

A hole pattern was formed while gradually decreasing the exposure dose from the optimum exposure dose at which the hole pattern can be formed, and in this event, the limit of the hole diameter (nm) at which resolution was possible was determined using a length-measurement SEM (CG-6300), manufactured by Hitachi High-Technologies Corporation, to specify this value as limiting resolution (nm). A smaller value indicates that it is possible to form a pattern having a finer hole diameter with better limiting resolution.

TABLE 6

| | | | | | | Limiting |
|---|---|---|---|---|---|---|
| | Resist | PAB/PEB | | Eop | LWR | resolution |
| | composition | ( ° C.) | Developer | (mJ/cm²) | (nm) | (nm) |
| Example 4-1 | R-01 | 130/90 | nBA | 16 | 2.1 | 20 |
| Example 4-2 | R-02 | 130/90 | nBA | 19 | 2.4 | 22 |
| Example 4-3 | R-03 | 130/90 | nBA | 19 | 2.2 | 21 |
| Example 4-4 | R-04 | 130/90 | nBA | 19 | 2.2 | 19 |
| Example 4-5 | R-05 | 130/90 | nBA | 23 | 2.2 | 19 |
| Example 4-6 | R-06 | 130/90 | nBA | 22 | 2.2 | 21 |
| Example 4-7 | R-07 | 130/90 | nBA | 20 | 2.1 | 23 |
| Example 4-8 | R-08 | 130/90 | nBA | 22 | 2.0 | 23 |
| Example 4-9 | R-09 | 130/90 | nBA | 19 | 2.0 | 23 |
| Example 4-10 | R-10 | 130/90 | nBA | 20 | 2.3 | 21 |
| Example 4-11 | R-11 | 130/90 | nBA | 15 | 1.8 | 22 |
| Example 4-12 | R-12 | 130/90 | nBA | 15 | 1.8 | 18 |
| Example 4-13 | R-13 | 130/90 | CHA | 14 | 1.9 | 18 |
| Example 4-14 | R-14 | 130/90 | nBA | 14 | 1.8 | 17 |
| Example 4-15 | R-15 | 130/90 | nBA | 16 | 1.8 | 18 |
| Example 4-16 | R-16 | 130/90 | nBA | 17 | 2.3 | 19 |
| Example 4-17 | R-17 | 130/90 | nBA | 16 | 2.0 | 22 |
| Example 4-18 | R-18 | 130/90 | nBA | 19 | 2.3 | 20 |
| Example 4-19 | R-19 | 130/90 | nBA | 16 | 2.3 | 21 |
| Example 4-20 | R-20 | 130/90 | nBA | 16 | 2.0 | 18 |
| Example 4-21 | R-21 | 130/90 | nBA | 15 | 2.1 | 19 |
| Example 4-22 | R-22 | 130/90 | nBA | 18 | 1.7 | 23 |
| Comparative | CR-01 | 130/90 | nBA | 25 | 2.7 | 26 |
| Example 4-1 | | | | | | |
| Comparative | CR-02 | 130/90 | nBA | 26 | 2.9 | 28 |
| Example 4-2 | | | | | | |
| Comparative | CR-03 | 105/90 | TMAH | 42 | 3.8 | 32 |
| Example 4-3 | | | | | | |
| Comparative | CR-04 | 105/90 | TMAH | 40 | 4.0 | 32 |
| Example 4-4 | | | | | | |

From the results shown in Table 6 above, it was found that the inventive resist composition was excellent in sensitivity, CDU, and resolution in contact hole pattern formation by EUV exposure.

The present description includes the following embodiments.

[1]: A hypervalent iodine compound having a structure represented by the following general formula (1),

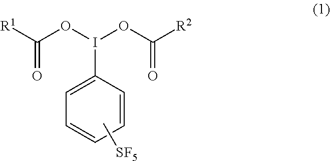

wherein R¹ and R² each independently represent a halogen atom or a hydrocarbyl group having 1 to 10 carbon atoms and optionally containing a heteroatom, the R¹ and the R² optionally being bonded to each other to form a ring together with the carbon atoms bonded thereto and the atoms between the carbon atoms.

[2]: A resist composition comprising: the hypervalent iodine compound according to [1]; a carboxy-group-containing compound; and a solvent.

[3]: The resist composition according to [2], wherein the carboxy-group-containing compound is a polymer including a repeating unit represented by the following general formula (2) and/or a compound represented by the following general formula (3),

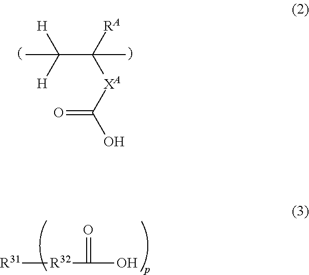

wherein R^{A} represents a hydrogen atom, a halogen atom, a methyl group, or a trifluoromethyl group;
X^{A} represents a single bond, a phenylene group, a naphthylene group, or *—C(═O)—O—X^{A1}—, X^{A1} representing a saturated hydrocarbylene group having 1 to 10 carbon atoms, a phenylene group, or a naphthylene group, the saturated hydrocarbylene group optionally containing a hydroxy group, an ether bond, an ester bond, or a lactone ring, and “*” representing an attachment point to the carbon atom of the main chain;
“p” represents 1, 2, 3, or 4;
R³¹ represents a p-valent hydrocarbon group having 1 to 40 carbon atoms or a p-valent heterocyclic group having 2 to 40 carbon atoms, when “p” is 2, the R³¹ optionally being an ether bond, a carbonyl group, an azo group, a thioether bond, a carbonate bond, a carbamate bond, a sulfinyl group, or a sulfonyl group, part or all of hydrogen atoms of the p-valent hydrocarbon group or the p-valent heterocyclic group optionally being substituted with a group containing a heteroatom, and part of —CH₂— of the p-valent hydrocarbon group optionally being substituted with a group containing a heteroatom; and
R³² represents a single bond or a hydrocarbylene group having 1 to 10 carbon atoms, part or all of hydrogen atoms of the hydrocarbylene group optionally being substituted with a group containing a heteroatom, part of —CH₂— of the hydrocarbylene group optionally being substituted with a group containing a heteroatom, and when “p” is 2, 3, or 4, the R³²s being identical to or different from each other.

[4]: The resist composition according to [2] or [3], further comprising at least one kind of hypervalent iodine compound represented by the following general formula (4) or (5),

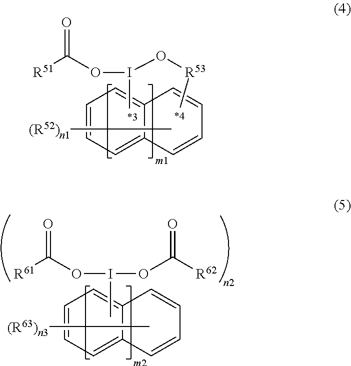

wherein “m1” and “m2” each represent an integer of 0 to 2, “n1” representing an integer of 0 to 4 when “m1” is 0, an integer of 0 to 6 when “m1” is 1, and an integer of 0 to 8 when “m1” is 2, when “m2” is 0, “n2” representing an integer of 1 to 3, “n3” representing an integer of 0 to 5, and 1≤(n2+n3)≤6 being satisfied, when “m2” is 1, “n2” representing an integer of 1 to 3, “n3” representing an integer of 0 to 7, and 1≤(n2+n3)≤8 being satisfied, and when “m2” is 2, “n2” representing an integer of 1 to 3, “n3” representing an integer of 0 to 9, and 1≤(n2+n3)≤10 being satisfied; R⁵¹ represents a halogen atom or a hydrocarbyl group having 1 to 10 carbon atoms and optionally containing a heteroatom; R⁵² represents a halogen atom or a hydrocarbyl group having 1 to 40 carbon atoms and optionally containing a heteroatom, when “n1” is 2 to 8, the R⁵²s being identical to or different from each other and the R⁵²s optionally being bonded to each other to form a ring together with the carbon atoms of the aromatic ring bonded to the R⁵²s; R⁵³ represents a hydrocarbylene group having 1 to 10 carbon atoms and optionally containing a heteroatom; “*3” and “*4” each represent an attachment point to one of the carbon atoms of the aromatic ring in the formula, provided that “*3” and “*4” are bonded to adjacent carbon atoms of the aromatic ring; R⁶¹ and R⁶² each independently represent a halogen atom or a hydrocarbyl group having 1 to 10 carbon atoms and optionally containing a heteroatom, the R⁶¹ and the R⁶² optionally being bonded to each other to form a ring together with the carbon atoms bonded thereto and the atoms between the carbon atoms, and when “n2” is 2 or 3, the Rols and the R⁶²s being identical to or different from each other; and R⁶³ represents a halogen atom or a hydrocarbyl group having 1 to 40 carbon atoms and optionally containing a heteroatom, when “n3” is 2 to 9, the R⁶³s being identical to or different from each other and the R⁶³s optionally being bonded to each other to form a ring together with the carbon atoms of the aromatic ring bonded to the R⁶³s.

[5]: A laminate comprising: a substrate; and a resist film, obtained from the resist composition according to any one of [2] to [4], on the substrate.

[6]: The laminate according to [5], comprising a resist underlayer film between the substrate and the resist film.

[7]: The laminate according to [5] or [6], wherein the resist film is a product formed by ligand exchange between the hypervalent iodine compound and the carboxy-group-containing compound.

[8]: A patterning process comprising the steps of:
forming a resist film by using the resist composition according to any one of [2] to [4] on a substrate or on a resist underlayer film of a substrate on which the resist underlayer film has been laminated;
exposing the resist film by using a high-energy beam; and
developing the exposed resist film by using a developer.

[9]: The patterning process according to [8], wherein the high-energy beam is an i-line, a KrF excimer laser beam, an ArF excimer laser beam, an electron beam, or an extreme ultraviolet ray.

[10]: The patterning process according to [8] or [9], wherein the developer dissolves exposed portions and does not dissolve unexposed portions.

[11]: The patterning process according to [8] or [9], wherein the developer dissolves unexposed portions and does not dissolve exposed portions.

It should be noted that the present invention is not limited to the above-described embodiments. The embodiments are just examples, and any examples that have substantially the same feature and demonstrate the same functions and effects as those in the technical concept disclosed in claims of the present invention are included in the technical scope of the present invention.

1. A hypervalent iodine compound having a structure represented by the following general formula (1), 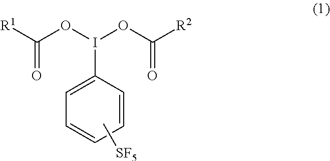
wherein R¹ and R² each independently represent a halogen atom or a hydrocarbyl group having 1 to 10 carbon atoms and optionally containing a heteroatom, the R¹ and the R² optionally being bonded to each other to form a ring together with the carbon atoms bonded thereto and the atoms between the carbon atoms.

2. A resist composition comprising: the hypervalent iodine compound according to claim 1; a carboxy-group-containing compound; and a solvent.

3. The resist composition according to claim 2, wherein the carboxy-group-containing compound is a polymer including a repeating unit represented by the following general formula (2) and/or a compound represented by the following general formula (3), 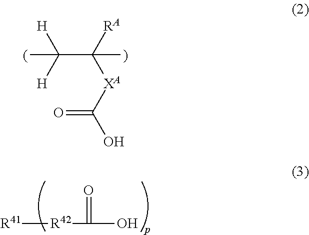
wherein R^{A} represents a hydrogen atom, a halogen atom, a methyl group, or a trifluoromethyl group;
X^{A} represents a single bond, a phenylene group, a naphthylene group, or *—C(═O)—O—X^{A1}—, X^{A1} representing a saturated hydrocarbylene group having 1 to 10 carbon atoms, a phenylene group, or a naphthylene group, the saturated hydrocarbylene group optionally containing a hydroxy group, an ether bond, an ester bond, or a lactone ring, and “*” representing an attachment point to the carbon atom of the main chain;
“p” represents 1, 2, 3, or 4;
R³¹ represents a p-valent hydrocarbon group having 1 to 40 carbon atoms or a p-valent heterocyclic group having 2 to 40 carbon atoms, when “p” is 2, the R³¹ optionally being an ether bond, a carbonyl group, an azo group, a thioether bond, a carbonate bond, a carbamate bond, a sulfinyl group, or a sulfonyl group, part or all of hydrogen atoms of the p-valent hydrocarbon group or the p-valent heterocyclic group optionally being substituted with a group containing a heteroatom, and part of —CH₂— of the p-valent hydrocarbon group optionally being substituted with a group containing a heteroatom; and
R³² represents a single bond or a hydrocarbylene group having 1 to 10 carbon atoms, part or all of hydrogen atoms of the hydrocarbylene group optionally being substituted with a group containing a heteroatom, part of —CH₂— of the hydrocarbylene group optionally being substituted with a group containing a heteroatom, and when “p” is 2, 3, or 4, the R³²s being identical to or different from each other.

4. The resist composition according to claim 2, further comprising at least one kind of hypervalent iodine compound represented by the following general formula (4) or (5), 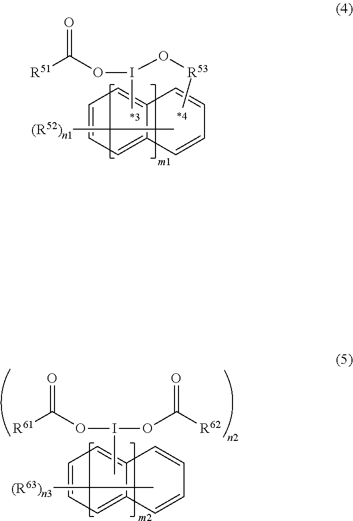
wherein “m1” and “m2” each represent an integer of 0 to 2, “n1” representing an integer of 0 to 4 when “m1” is 0, an integer of 0 to 6 when “m1” is 1, and an integer of 0 to 8 when “m1” is 2, when “m2” is 0, “n2” representing an integer of 1 to 3, “n3” representing an integer of 0 to 5, and 1≤(n2+n3)≤6 being satisfied, when “m2” is 1, “n2” representing an integer of 1 to 3, “n3” representing an integer of 0 to 7, and 1≤(n2+n3)≤8 being satisfied, and when “m2” is 2, “n2” representing an integer of 1 to 3, “n3” representing an integer of 0 to 9, and 1≤(n2+n3)≤10 being satisfied; R⁵¹ represents a halogen atom or a hydrocarbyl group having 1 to 10 carbon atoms and optionally containing a heteroatom; R⁵² represents a halogen atom or a hydrocarbyl group having 1 to 40 carbon atoms and optionally containing a heteroatom, when “n1” is 2 to 8, the R⁵²s being identical to or different from each other and the R⁵²s optionally being bonded to each other to form a ring together with the carbon atoms of the aromatic ring bonded to the R⁵²s; R⁵³ represents a hydrocarbylene group having 1 to 10 carbon atoms and optionally containing a heteroatom; “*3” and “*4” each represent an attachment point to one of the carbon atoms of the aromatic ring in the formula, provided that “*3” and “*4” are bonded to adjacent carbon atoms of the aromatic ring; R⁶¹ and R⁶² each independently represent a halogen atom or a hydrocarbyl group having 1 to 10 carbon atoms and optionally containing a heteroatom, the R⁶¹ and the R⁶² optionally being bonded to each other to form a ring together with the carbon atoms bonded thereto and the atoms between the carbon atoms, and when “n2” is 2 or 3, the Rols and the R⁶²s being identical to or different from each other; and R⁶³ represents a halogen atom or a hydrocarbyl group having 1 to 40 carbon atoms and optionally containing a heteroatom, when “n3” is 2 to 9, the R⁶³s being identical to or different from each other and the R⁶³s optionally being bonded to each other to form a ring together with the carbon atoms of the aromatic ring bonded to the R⁶³s.

5. A laminate comprising: a substrate; and a resist film, obtained from the resist composition according to claim 2, on the substrate.

6. A laminate comprising: a substrate; and a resist film, obtained from the resist composition according to claim 3, on the substrate.

7. A laminate comprising: a substrate; and a resist film, obtained from the resist composition according to claim 4, on the substrate.

8. The laminate according to claim 5, comprising a resist underlayer film between the substrate and the resist film.

9. The laminate according to claim 5, wherein the resist film is a product formed by ligand exchange between the hypervalent iodine compound and the carboxy-group-containing compound.

10. A patterning process comprising the steps of:
forming a resist film by using the resist composition according to claim 2 on a substrate or on a resist underlayer film of a substrate on which the resist underlayer film has been laminated;
exposing the resist film by using a high-energy beam; and
developing the exposed resist film by using a developer.

11. A patterning process comprising the steps of:
forming a resist film by using the resist composition according to claim 3 on a substrate or on a resist underlayer film of a substrate on which the resist underlayer film has been laminated;
exposing the resist film by using a high-energy beam; and
developing the exposed resist film by using a developer.

12. A patterning process comprising the steps of:
forming a resist film by using the resist composition according to claim 4 on a substrate or on a resist underlayer film of a substrate on which the resist underlayer film has been laminated;
exposing the resist film by using a high-energy beam; and
developing the exposed resist film by using a developer.

13. The patterning process according to claim 10, wherein the high-energy beam is an i-line, a KrF excimer laser beam, an ArF excimer laser beam, an electron beam, or an extreme ultraviolet ray.

14. The patterning process according to claim 10, wherein the developer dissolves exposed portions and does not dissolve unexposed portions.

15. The patterning process according to claim 10, wherein the developer dissolves unexposed portions and does not dissolve exposed portions.
